(19) United States
(12) Patent Application Publication
(10) Pub. No.: US 2011/0166020 A1
(43) Pub. Date: Jul. 7, 2011

(54) Imidazole and Triazole Compounds, Use Thereof and Agents Containing Said Compounds

(75) Inventors: Jens Renner, Bad Duerkheim (DE); Jochen Dietz, Karlsruhe (DE); Thomas Grote, Wachenheim (DE); Bernd Müller, Frankenthal (DE); Jan Klaas Lohmann, Mannheim (DE); Sarah Ulmschneider, Bad Duerkheim (DE); Alice Glättli, Frankfurt (DE); Marianna Vrettou, Mannheim (DE); Wassilios Grammenos, Ludwigshafen (DE)

(73) Assignee: BASF SE, Ludwigshafen (DE)

(21) Appl. No.: 13/062,548

(22) PCT Filed: Sep. 1, 2009
(86) PCT No.: PCT/EP2009/061230

§ 371 Date: Mar. 7, 2011

(30) Foreign Application Priority Data
Sep. 10, 2008 (EP) 08164043.5

(51) Int. Cl. A01N 25/26; C07D 249/08; A01N 43/653; A01P 3/00

(52) U.S. Cl. 504/100; 548/262.2; 514/383

(57) ABSTRACT

The present invention relates to compounds of the formula I

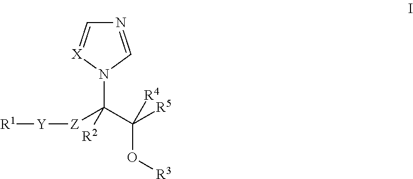

in which the variables have the meanings defined in the claims and in the description.

The present invention relates to imidazole and triazole compounds of the formula I

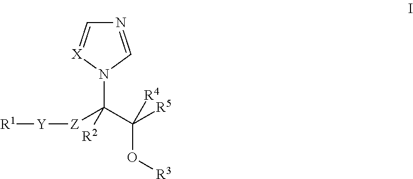

in which the variables have the following meanings:
X is CH or N;
Y is O or a single bond to R¹;
Z is a saturated or partially unsaturated hydrocarbon chain which has two to eight carbon atoms and which, if it is partially unsaturated, comprises one to three double bonds or one or two triple bonds, where Z may comprise one, two, three, four or five substituents R^{Z}, where R^{Z} is as defined below:
R^{Z} is halogen, cyano, nitro, cyanato (OCN), C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₃-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, C₁-C₆-alkylene, oxy-C₂-C₄-alkylene, oxy-C₁-C₃-alkyleneoxy, phenoxy, phenyl, heteroaryloxy, heterocyclyloxy, heteroaryl, heterocyclyl, where in the groups mentioned above the heteroaryl is an aromatic five-, six- or seven-membered heterocycle and the heterocyclyl is a saturated or partially unsaturated five-, six- or seven-membered heterocycle, each of which contains one, two, three or four heteroatoms from the group consisting of O, N and S, or is NA³A⁴, where two radicals R^{Z} attached to the same carbon atom, together with the carbon atom to which they are attached, may also form a C₃-C₆-cycloalkyl ring; where A³, A⁴ are as defined below;
R¹ is a three-, four-, five-, six-, seven-, eight-, nine- or ten-membered saturated or partially unsaturated heterocycle or a five-, six-, seven-, eight-, nine- or ten-membered aromatic heterocycle, where the heterocycle contains in each case one, two, three or four heteroatoms from the group consisting of O, N and S, where the heterocycle is unsubstituted or contains one, two, three, four or five independently selected substituents L, where L is as defined below:
   L is halogen, cyano, nitro, hydroxyl, cyanato (OCN), C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₃-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₈-cyclo-alkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenoxy, phenyl, heteroaryloxy, heterocyclyloxy, heteroaryl, heterocyclyl, where in the groups mentioned above the heteroaryl is an aromatic five-, six- or seven-membered heterocycle and the heterocyclyl is a saturated or partially unsaturated five-, six- or seven-membered heterocycle, each of which contains one, two, three or four heteroatoms from the group consisting of O, N and S, where n, A¹, A², A³, A⁴ are as defined below:
   n is 0, 1 or 2;
   A¹ is hydrogen, hydroxyl, C₁-C₈-alkyl, C₁-C₈-haloalkyl, amino, C₁-C₈-alkylamino, di-C₁-C₈-alkylamino, phenyl, phenylamino or phenyl-C₁-C₈-alkylamino;
   A² is one of the groups mentioned for A¹ or is C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₃-C₈-haloalkynyloxy, C₃-C₈-cycloalkyl, C₃-C₈-halocyclo-alkyl, C₃-C₈-cycloalkoxy or C₃-C₈-halocycloalkoxy;
   A³,A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₃-C₈-cycloalkyl, C₃-C₈-halocycloalkyl, C₃-C₈-cycloalkenyl or C₃-C₈-halocycloalkenyl, phenyl or 5- or six-membered heteroaryl having one, two, three or four heteroatoms from the group consisting of O, N and S in the heterocycle;
the aliphatic and/or alicyclic and/or aromatic groups of the radical definitions of L for their part may carry one, two, three or four identical or different groups R^{L}:
   R^{L} is halogen, hydroxyl, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₆-alkylene, oxy-C₂-C₄-alkylene, oxy-C₁-C₃-alkyleneoxy, C₁-C₈-alkyl-carbonyl, C₁-C₈-alkylcarbonyloxy, C₁-C₈-alkoxycarbonyl, amino, C₁-C₈-alkylamino, di-C₁-C₈-alkylamino;
R² is hydrogen, C₁-C₁₀-alkyl, C₁-C₁₀-haloalkyl, C₂-C₁₀-alkenyl, C₂-C₁₀-halo-alkenyl, C₂-C₁₀-alkynyl, C₃-C₁₀-haloalkynyl, C₄-C₁₀-alkadienyl, C₄-C₁₀-halo-alkadienyl, C₃-C₁₀-cycloalkyl, C₃-C₁₀-halocycloalkyl, C₃-C₁₀-cycloalkenyl, C₃-C₁₀-halocycloalkenyl;
R³ is hydrogen, C₁-C₁₀-alkyl, C₁-C₁₀-haloalkyl, C₂-C₁₀-alkenyl, C₂-C₁₀-halo-alkenyl, C₂-C₁₀-alkynyl, C₃-C₁₀-haloalkynyl, C₄-C₁₀-alkadienyl, C₄-C₁₀-halo-alkadienyl, C₃-C₁₀-cycloalkyl, C₃-C₁₀-halocycloalkyl, C₃-C₁₀-cycloalkenyl, C₃-C₁₀-halocycloalkenyl, carboxyl, formyl, Si(A⁵A⁶A⁷), C(O)Rⁿ, C(O)ORⁿ, C(S)ORⁿ, C(O)SRⁿ, C(S)SRⁿ, C(NR^{A})SRⁿ, C(S)Rⁿ, C(NRⁿ)N-NA³A⁴, C(NRⁿ)R^{A}, C(NRⁿ)OR^{A}, C(O)NA³A⁴, C(S)NA³A⁴ or S(═O)ₙA¹; where
   Rⁿ is C₁-C₈-alkyl, C₃-C₈-alkenyl, C₃-C₈-alkynyl, C₃-C₆-cycloalkyl, C₃-C₆-cycloalkenyl or phenyl;
   R^{A} is C₁-C₈-alkyl, C₃-C₈-alkenyl, C₃-C₈-alkynyl, C₃-C₆-cycloalkyl, C₃-C₆-cycloalkenyl or phenyl;
   A⁵, A⁶, A⁷ independently of one another are C₁-C₁₀-alkyl, C₃-C₈-alkenyl, C₃-C₈-alkynyl, C₃-C₆-cycloalkyl, C₃-C₆-cycloalkenyl or phenyl;
   where Rⁿ, R^{A}, A⁵, A⁶ and A⁷ are, unless indicated otherwise, independently of one another unsubstituted or substituted by one, two, three, four or five L, as defined above;
R⁴ is hydrogen, C₁-C₁₀-alkyl, C₁-C₁₀-haloalkyl, C₂-C₁₀-alkenyl, C₂-C₁₀-halo-alkenyl, C₂-C₁₀-alkynyl, C₃-C₁₀-haloalkynyl, C₄-C₁₀-alkadienyl, C₄-C₁₀-halo-alkadienyl, C₃-C₁₀-cycloalkyl, C₃-C₁₀-halocycloalkyl, C₃-C₁₀-cycloalkenyl, C₃-C₁₀-halocycloalkenyl;
   R², R³, R⁴ are, unless indicated otherwise, independently of one another unsubstituted or substituted by one, two, three, four or five L, as defined above;
R⁵ C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, C₃-C₁₀-cycloalkenyl, C₃-C₁₀-halocycloalkenyl, phenyl, five-, six- or seven-membered heteroaryl which contains 1, 2, 3 or 4 heteroatoms from the group consisting of O, N and S or five-, six- or seven-membered saturated or partially unsaturated heterocyclyl which contains 1, 2, 3 or 4 heteroatoms from the group consisting of O, N and S, where R⁵ may contain one, two, three, four, five or six independently selected substituents L, as defined above;
and agriculturally acceptable salts thereof.

The invention furthermore relates to the preparation of the compounds I, to the intermediates for preparing the compounds I and to their preparation, and also to the use of the compounds according to the invention for controlling phytopathogenic fungi, and to compositions comprising them.

Imidazole and triazole compounds are known from GB 2 081 709 and DE 3215360.

However, in particular at low application rates, the fungicidal action of the compounds known from the prior art is sometimes unsatisfactory. Accordingly, it was an object of the present invention to provide novel compounds which preferably have improved properties, such as improved fungicidal action and/or better toxicological properties. Surprisingly, this object was achieved with the compounds of the formula I described here.

Owing to the basic character of their nitrogen atoms, the compounds I are capable of forming salts or adducts with inorganic or organic acids or with metal ions. This also applies to most of the precursors described herein of compounds I, the salts and adducts of which are also provided by the present invention.

Examples of inorganic acids are hydrohalic acids, such as hydrogen fluoride, hydrogen chloride, hydrogen bromide and hydrogen iodide, carbonic acid, sulfuric acid, phosphoric acid and nitric acid.

Suitable organic acids are, for example, formic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals with 1 to 20 carbon atoms), arylphosphonic acids or aryldiphosphonic acids (aromatic radicals, such as phenyl and naphthyl, which carry one or two phosphoric acid radicals), where the alkyl or aryl radicals may carry further substituents, for example p-toluenesulfonic acid, salicylic acid, p-aminosalicylic acid, 2-phenoxybenzoic acid, 2-acetoxybenzoic acid etc.

Suitable metal ions are in particular the ions of the elements of the second main group, in particular calcium and magnesium, of the third and fourth main group, in particular aluminum, tin and lead and also of the elements of transition groups one to eight, in particular chromium, manganese, iron, cobalt, nickel, copper, zinc and others. Particular preference is given to the metal ions of the elements of transition groups of the fourth period. The metals can be present in the various valencies that they can assume.

The compounds I according to the invention can be prepared by different routes analogously to processes known per se of the prior art (see, for example, the prior art cited at the outset). The compounds according to the invention can be prepared, for example, according to the syntheses shown in the schemes below.

Thus, a compound of the formula I in which R³ and R⁴ are hydrogen (compounds I-1)

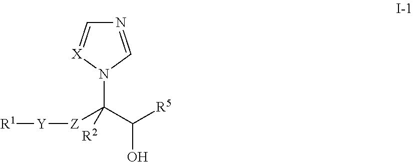

where R¹, X, Y, Z, R² and R⁵ are as defined or as preferably defined for formula I can be prepared from compounds II by reducing the keto group

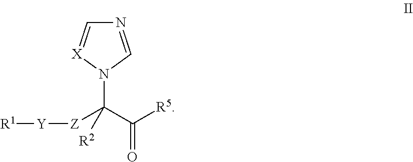

Reducing agents that can be used are, for example, borohydrides, in particular sodium borohydride, potassium borohydride, tetra-n-butylammonium borohydride and other metal hydrides. It may be advantageous to add an additive such as, for example, a Lewis acid, generally in substoichiometric or stoichiometric amounts. Titanium halides such as titanium tetrachloride, titanium alkoxides such as titanium tetraisopropoxide or zinc halides or tin halides such as zinc chloride or tin chloride have been found to be particularly suitable. See, for example, Chem Ber 121(6), 1988, 1059 ff. Other suitable reducing agents are, for example, alkylmagnesium halides, such as, for example, isopropylmagnesium chloride or tert-butylmagnesium chloride. See, for example, DE3511922, DE3437919, DE3415486, DE3600812.

The present invention furthermore provides compounds of the formula II

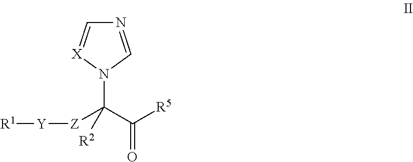

in which R¹, X, Y, Z, R² and R⁵ are as defined or as preferably defined for formula I. According to one embodiment, R² is hydrogen (compounds II-1).

Compounds of the formula II can be obtained by alkylation reactions, for example by reacting a compound of the formula III

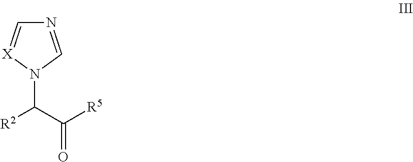

with a compound IV

R¹—Y-Z-LG (IV)

and a base, where LG is a leaving group such as, for example, halogen, in particular Cl, Br and I, or mesylate, tosylate, or another suitable leaving group known to the person skilled in the art. R¹, Y and Z have the meanings or preferred meanings as defined for formula I. Suitable bases are alkali metal or alkaline earth metal hydrides, alkali metal amides or alkoxides.

Processes for preparing compounds of type IV are known to the person skilled in the art.

Alternatively, compounds I-1 can be obtained from compounds V

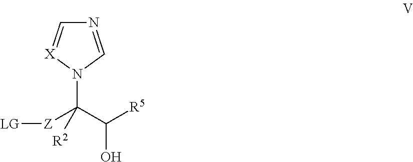

in which X, Z, R² and R⁵ are as defined or as preferably defined for formula I and LG is a leaving group (for example, see above) or OH which, if Z comprises one or more multiple bonds, is not attached directly to a multiple bond. Here, LG is substituted by a nucleophile such as, for example, an alkoxide, or by the nitrogen of a heteroaromatic to introduce a group R¹—Y. To this end, for example, a corresponding alcohol R¹—OH, such as in particular an optionally substituted phenol, is initially charged in an inert solvent, and a base is added to the reaction mixture. Suitable bases are carbonates, alkali metal or alkaline earth metal hydrides, alkali metal amides and alkoxides. The reaction is generally carried out in a temperature range between 20° C. and 150° C. Alternatively, an optionally substituted heterocycle, preferably a substituted heteroaromatic, can be initially charged in an inert solvent, and a base can be added. Here, suitable bases are alkali metal or alkaline earth metal alkyls, such as, for example, n-butyllithium, methyllithium or tert-butyllithium, or alkali metal or alkaline earth metal hydrides, such as, for example, sodiumhydride or potassium hydride. It is also possible to use alkali metal amides, such as sodium amide, lithium diisopropylamide or lithium hexamethyldisilazide, or alkoxides. The LG can also be generated, for example, from an OH group using processes known to the person skilled in the art. An example which may be mentioned here are reactions with sulfonyl chlorides or sulfonyl anhydrides (methanesulfonyl, trifluoromethylsulfonyl or tosyl) in the presence of an amine base, or reactions with halogenating agents such as, for example, PBr₃ or HBr.

Compounds V can be obtained by reducing the keto group in the corresponding keto compound VI

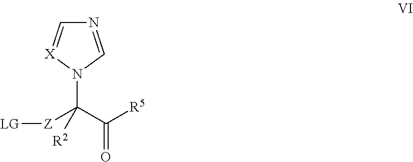

as described above to the alcohol.

Compounds VI can be prepared by reacting a compound III (see above) with a compound VII

LG-Z-LG (VII)

in which Z is as defined or as preferably defined for formula I and LG is in each case independently a leaving group (for examples, see above) and one of the two groups LG may also be OH, where the leaving groups, if Z contains one or more multiple bonds, are not attached directly to a multiple bond. Z is in particular an alkylene chain #—[CH₂]ₙ—# in which # indicates the points of attachment and n is from 2 to 8, in particular from 3 to 5. Suitable bases are alkali metal or alkaline earth metal hydrides, alkali metal amides and alkoxides. The temperature range of these reactions is preferably between −20° C. and 160° C., and the reaction is carried out in particular in solvents such as DMF, NMP, cyclic or acyclic ethers.

According to a further process, compounds I (where R³=hydrogen) can be obtained by reacting an oxirane of the formula XI

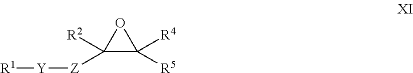

in which Y, Z, R¹, R², R⁴ and R⁵ are as defined or as preferably defined for formula I, with imidazole or triazole in the presence of a base with opening of the epoxide to form the target products. Such processes are described, for example, in EP 0 236 884.

The oxirane XI can be obtained by reacting the corresponding olefin XII

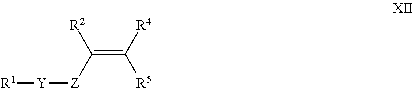

with a peracid or an equivalent reagent (such as, for example, dimethyldioxirane or other peroxides, see also EP 0 236 884).

The olefin XII can be prepared by a Wittig reaction from

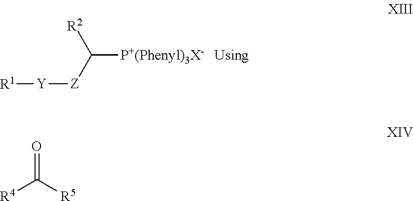

(see also EP 0 236 884).

Alternatively, olefins XII can be prepared via the corresponding alcohol XV

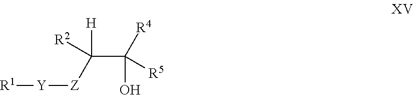

which, in an elimination reaction familiar to the person skilled in the art, is converted into the olefin (see also EP 0 236 884). The preparation of the alcohols XV is described, for example, in DE 3400829. Optionally, the double bond may be isomerized to obtain the desired configuration of the oxirane. Processes for achieving this will be familiar to the person skilled in the art.

To obtain, starting with compounds of the formula I-1, compounds where R³≠hydrogen (compounds I-3),

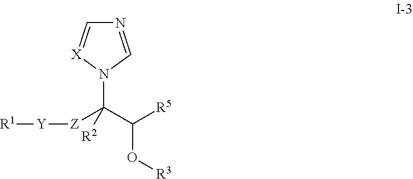

methods, known to the person skilled in the art, for alkylating, esterifying etc. of alcohols may be employed (see also DE 3321422, DE 3019049).

To prepare compounds of the formula I in which R⁴≠hydrogen (compounds I-4),

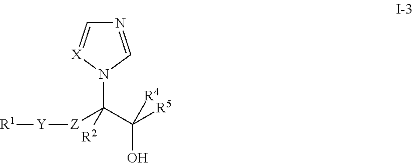

a procedure analogous to the processes described in DE 3126022, DE 3049542 may be adopted, and the corresponding ketone of the formula II (see above) can be converted with a Grignard reagent (R⁴—Mg—HaI) into the corresponding tertiary alcohol.

In a corresponding manner, it is also possible to prepare compounds I in which two or three substituents of R², R³ and R⁴ are not hydrogen, by combining the processes mentioned with one another.

In some of the definitions of the symbols in the formulae given herein, collective terms are used which are generally representative of the following substituents:

halogen: fluorine, chlorine, bromine and iodine;
alkyl and the alkyl moieties of composite groups such as, for example, alkylamino: saturated straight-chain or branched hydrocarbon radicals having 1 to 4, 6, 8 or 12 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethyl-propyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: alkyl as mentioned above, where some or all of the hydrogen atoms in these groups are replaced by halogen atoms as mentioned above; in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoro-prop-2-yl
alkenyl and also the alkenyl moieties in composite groups, such as alkenyloxy: unsaturated straight-chain or branched hydrocarbon radicals having 2 to 4, 2 to 6 or 2 to 8 carbon atoms and one double bond in any position. According to the invention, it may be preferred to use small alkenyl groups, such as (C₂-C₄)-alkenyl; on the other hand, it may also be preferred to employ larger alkenyl groups, such as (C₅-C₈)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: alkenyl as defined above, where some or all of the hydrogen atoms in these groups are replaced by halogen atoms as described above under haloalkyl, in particular by fluorine, chlorine or bromine;
alkadienyl: unsaturated straight-chain or branched hydrocarbon radicals having 4 to 6 or 4 to 8 carbon atoms and two double bonds in any position;
alkynyl and the alkynyl moieties in composite groups: straight-chain or branched hydrocarbon groups having 2 to 4, 2 to 6 or 2 to 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
haloalkynyl: alkynyl as defined above, where some or all of the hydrogen atoms in these groups are replaced by halogen atoms as described above under haloalkyl, in particular by fluorine, chlorine or bromine;
cycloalkyl and also the cycloalkyl moieties in composite groups: mono- or bicyclic saturated hydrocarbon groups having 3 to 8, in particular 3 to 6, carbon ring members, for example C₃-C₆-cycloalkyl, such as cyclopropyl, cyclobutyl, cyclopentyl, cyclohexyl;
halocycloalkyl: cycloalkyl as defined above, where some or all of the hydrogen atoms in these groups are replaced by halogen atoms as described above under haloalkyl, in particular by fluorine, chlorine or bromine;
cycloalkenyl: monocyclic monounsaturated hydrocarbon groups having preferably 3 to 8 or 4 to 6, in particular 5 to 6, carbon ring members, such as cyclopenten-1-yl, cyclopenten-3-yl, cyclohexen-1-yl, cyclohexen-3-yl, cyclohexen-4-yl and the like;
halocycloalkenyl: cycloalkenyl as defined above, where some or all of the hydrogen atoms in these groups are replaced by halogen atoms as described above under haloalkyl, in particular by fluorine, chlorine or bromine;
alkoxy: an alkyl group as defined above which is attached via an oxygen, preferably having 1 to 8, more preferably 2 to 6, carbon atoms. Examples are: methoxy, ethoxy, n-propoxy, 1-methylethoxy, butoxy, 1-methylpropoxy, 2-methylpropoxy or 1,1-dimethylethoxy, and also for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;
haloalkoxy: alkoxy as defined above, where some or all of the hydrogen atoms in these groups are replaced by halogen atoms as described above under haloalkyl, in particular by fluorine, chlorine or bromine; Examples are OCH₂F, OCHF₂, OCF₃, OCH₂Cl, OCHCl₂, OCCl₃, chlorofluoromethoxy, dichlorofluoromethoxy, chlorodifluoro-methoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C2F₅, 1-(CH₂F)-2-fluoroethoxy, 1-(CH₂Cl)-2-chloroethoxy, 1-(CH₂Br)-2-bromo-ethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.
alkylene: divalent unbranched chains of CH₂ groups. Preference is given to (C₁-C₆)-alkylene, more preference to (C₂-C₄)-alkylene; furthermore, it may be preferred to use (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₄-CH₂;
6- to 10-membered aryl: an aromatic hydrocarbon cycle having 6, 7, 8, 9 or 10 carbon atoms in the ring, in particular phenyl or naphthyl.
a 3-, 4-, 5-, 6-, 7-, 8-, 9- or 10-membered saturated or partially unsaturated heterocycle which contains 1, 2, 3 or 4 heteroatoms from the group consisting of O, N and S, where the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. In particular:
a three- or four-membered saturated heterocycle (hereinbelow also referred to as heterocyclyl) which contains one or two heteroatoms from the group consisting of O, N and S as ring members;
a five- or six-membered saturated or partially unsaturated heterocycle which contains one, two, three or four heteroatoms from the group consisting of O, N and S as ring members: for example monocyclic saturated or partially unsaturated heterocycles which, in addition to carbon ring members, contain one, two or three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydro-oxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexa-hydropyridazinyl, 2-hexahydropyrimidinyl, 4-hexahydropyrimidinyl, 5-hexahydro-pyrimidinyl, 2-piperazinyl, 1,3,5-hexahydrotriazin-2-yl and 1,2,4-hexahydrotriazin-3-yl and also the corresponding-ylidene radicals; a seven-membered saturated or partially unsaturated heterocycle which contains one, two, three or four heteroatoms from the group consisting of O, N and S as ring members: for example mono- and bicyclic heterocycles having 7 ring members which, in addition to carbon ring members, contain one, two or three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetra-hydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl such as 2,3,4,5-tetrahydro[1 H]oxepin-2-, -3-, -4-, -5-, -6- or -7-yl, 2,3,4,7-tetra-hydro[1 H]oxepin-2-, -3-, -4-, -5-, -6- or -7-yl, 2,3,6,7-tetrahydro[1 H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals;
   a 5-, 6-, 7-, 8-, 9- or 10-membered aromatic heterocycle which contains 1, 2, 3 or 4 heteroatoms from the group consisting of O, N and S: in particular a five- or six-membered aromatic mono- or bicyclic heterocycle which contains one, two, three or four heteroatoms from the group consisting of O, N and S: the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. The heterocycle is in particular:
5-membered heteroaryl which contains one, two, three or four nitrogen atoms or one, two or three nitrogen atoms and/or one sulfur or oxygen atom, where the heteroaryl may be attached via carbon or nitrogen, if present: 5-membered heteroaryl groups which, in addition to carbon atoms, may comprise one to four nitrogen atoms or one, two or three nitrogen atoms and/or one sulfur or oxygen atom as ring members, for example furyl, thienyl, pyrrolyl, pyrazolyl, imidazolyl, triazolyl (1,2,3-; 1,2,4-triazolyl), tetrazolyl, oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
6-membered heteroaryl which comprises one, two, three or four, preferably one, two or three, nitrogen atoms, where the heteroaryl may be attached via carbon or nitrogen, if present: 6-membered heteroaryl groups which, in addition to carbon atoms, may comprise one to four or one, two or three nitrogen atoms as ring members, for example pyridinyl, pyrimidinyl, pyrazinyl, pyridazinyl, 1,2,3-triazinyl, 1,2,4-triazinyl, 1,3,5-triazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The novel compounds according to the invention comprise chiral centers and are generally obtained in the form of racemates or as diastereomer mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers. Suitable for use as antimicrobial agents are both the uniform diastereomers or enantiomers and mixtures thereof obtained in the synthesis. This applies correspondingly to the fungicidal compositions.

Accordingly, the invention provides both the pure enantiomers or diastereomers and mixtures thereof. This applies to the compounds according to the invention of the formula I and, optionally, correspondingly to their precursors. The scope of the present invention includes in particular the (R) and (S) isomers and the racemates of the compounds according to the invention, in particular of the formula I, which have centers of chirality. Suitable compounds according to the invention, in particular of the formula I, also comprise all possible stereoisomers (cis/trans isomers) and mixtures thereof.

Any double bonds in the variable Z in the compounds according to the invention may in each case be either (E)- or (Z)-configured. The present invention provides both the (E)- and the (Z)-isomers.

The compounds according to the invention, in particular of the formula I, may be present in various crystal modifications which may differ in their biological activity. They are likewise provided by the present invention.

In the compounds I according to the invention, particular preference is given to the following meanings of the substituents, in each case on their own or in combination.

According to one embodiment, X═N (triazole compounds of the formula I.A).

According to a further embodiment, X═CH (imidazole compounds of the formula I.B).

According to one embodiment of the invention, Y is O. According to a further embodiment of the invention, Y is a single bond between R¹ and Z.

In the compounds according to the invention, Z is a saturated or partially unsaturated hydrocarbon chain which has two, three, four, five, six, seven or eight carbon atoms and which, if it is partially unsaturated, comprises one, two or three double bonds or one or two triple bonds, where Z may comprise one, two, three, four or five substituents R^{Z}.

According to one embodiment, Z is a saturated hydrocarbon chain having three to eight carbon atoms, where Z is unsubstituted or may comprise one, two, three, four or five substituents R^{Z}.

According to one embodiment of the invention, Z is a group Z¹:

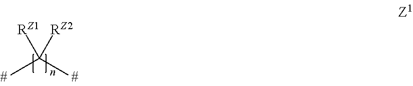

in which # are the points of attachment, n is 2, 3, 4, 5 or 6 and R^{z1} and R^{z2} are in each case independently of one another selected from the group consisting of hydrogen and R^{Z}, as defined herein.

According to one embodiment, n in the group Z¹ is 2.

According to one embodiment, n in the group Z¹ is 3. According to a specific embodiment, Y is simultaneously a bond.

According to a further embodiment, n in the group Z¹ is 4. According to a specific embodiment, Y is simultaneously O.

According to a further embodiment, n in the group Z¹ is 5.

According to a further embodiment, Z is Z¹ where n=3, 4 or 5 and Y is a single bond between R¹ and Z.

In a specific aspect of the respective embodiments mentioned, R^{z1} and R^{z2} are each independently of one another selected from the group consisting of hydrogen and R^{z}, as defined herein, where R^{z} is in particular selected from the group consisting of C₁-Cₐ-alkyl and C₃-C₆-cycloalkyl, and/or R^{z1} and R^{z2} together with the carbon to which they are attached form a C₃-C₆-cycloalkyl ring. In a further aspect, R^{z} is selected from the group consisting of F and Cl.

In a specific embodiment of the invention, all R^{z1} and R^{z2} in Z¹ are hydrogen.

According to a further embodiment, Z is a partially unsaturated hydrocarbon chain which has two to eight, in particular four to six, carbon atoms, and which comprises one to three double bonds, where Z may comprise one, two, three, four or five substituents R^{z}. According to one aspect, the hydrocarbon chain has a double bond. According to a further aspect, the hydrocarbon chain has two double bonds.

According to a further embodiment of the invention. Z is a aroma Z²

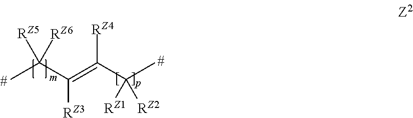

in which # are the points of attachment, m and p are each 0, 1 or 2, where m+p≧1, in particular m+p≧2, and R^{Z1}, R^{Z2}, R^{Z3}, R^{Z4}, R^{Z5} and R^{Z6} are in each case independently of one another selected from the group consisting of hydrogen and R^{Z}, where R^{z} is in each case as defined herein or as defined as being preferred.

According to one embodiment, m+p in group Z² is 1, where in particular m=0 and p=1.

According to a further embodiment, m+p in group Z² is 2, where in particular m=1 and p=1.

According to a further embodiment, m+p in group Z² is 3, where in particular m=0 and p=3.

In a specific aspect of the respective embodiments mentioned, R^{z3} and R^{z4} are independently of one another selected from the group consisting of hydrogen and R^{z}, as defined herein, where R^{z} is in particular selected from C₁-C₄-alkyl, in particular methyl or ethyl. R^{z1}, R^{z2}, R^{z5} and R^{z6} are preferably each independently of one another selected from the group consisting of hydrogen and C₁-C₄-alkyl and/or two radicals at a carbon atom form together with the carbon atom to which they are attached a C₃-C₆-cycloalkyl ring.

According to a further embodiment, R^{Z3} is hydrogen and R^{Z4} is selected from R^{z}. According to one aspect, R^{Z4} is C₁-C₄-alkyl, in particular methyl. According to a further aspect, R^{Z4} is halogen, in particular chlorine.

According to a further embodiment, R^{Z4} is hydrogen and R^{Z3} is selected from R^{z}. According to one aspect, R^{Z3} is C₁-C₄-alkyl, in particular methyl. According to a further aspect, R^{Z3} is halogen, in particular chlorine.

According to a further embodiment, R^{Z3} and R^{Z4} are independently of one another selected from R^{z}. According to one aspect, R^{Z4} and R^{Z5} are C₁-C₄-alkyl, in particular methyl. According to a further aspect, R^{Z3} is halogen, in particular chlorine.

According to one embodiment, R^{Z1}, R^{Z2}, R^{Z5} and R^{Z6} are all hydrogen. According to a further embodiment, R^{Z1}, R^{Z2}, R^{Z5} and R^{Z6} are independently of one another selected from the group consisting of hydrogen and halogen (in particular F and Cl), where at least one R^{z} is not hydrogen.

The double bond in the group Z² can be (E)- or (Z)-configured. The present invention provides both the (E)- and the (Z)-isomers. According to one embodiment, the double bond is (E)-configured. According to a further embodiment, the double bond is (Z)-configured.

According to a further embodiment, Z is a partially unsaturated hydrocarbon chain which has three to eight, in particular four to six, carbon atoms and which comprises one or two triple bonds, where Z may comprise one, two, three, four or five substituents R^{Z}. According to one aspect, the hydrocarbon chain has a triple bond. According to a further aspect, the hydrocarbon chain has two triple bonds.

According to a further embodiment of the invention, Z is a group Z³

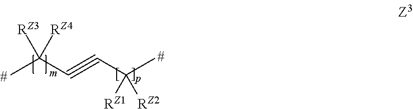

in which # are the points of attachment, m and p are each 0, 1 or 2, where m+p≧1, preferably m+p≧2, and R^{Z1}, R^{Z2}, R^{Z3} and R^{Z4} are in each case independently of one another selected from the group consisting of hydrogen and R^{Z}, where R^{z} is in each case as defined herein or as defined as being preferred.

According to one embodiment, m+p in group Z³ is 2, where in particular m=1 and p=1.

In a specific aspect of the respective embodiments mentioned, R^{z1}, R^{z2}, R^{z3} and R^{z4} are independently of one another selected from the group consisting of hydrogen and R^{z}, as defined herein, where R^{z} is in particular selected from C₁-C₄-alkyl, in particular methyl or ethyl.

The substituent(s) R^{z} at Z or in the group Z¹, Z² and Z³ is/are, unless indicated otherwise, in each case independently of one another selected from the group consisting of halogen, cyano, nitro, cyanato (OCN), C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₃-C₈-haloalkynyloxy, C₃-C₈-cycloalkyl, C₃-C₈-halocycloalkyl, C₃-C₈-cycloalkenyl, C₃-C₈-halocycloalkenyl, C₆-C₈-cycloalkynyl, C₆-C₈-halocycloalkynyl, C₃-C₈-cycloalkoxy, C₃-C₆-cycloalkenyloxy, C₁-C₆-alkylene, oxy-C₂-C₄-alkylene, oxy-C₁-C₃-alkyleneoxy, phenoxy, phenyl, heteroaryloxy, heterocyclyloxy, heteroaryl, heterocyclyl, where in the groups mentioned above the heteroaryl is an aromatic five-, six- or seven-membered heterocycle and the heterocyclyl is a saturated or partially unsaturated five-, six- or seven-membered heterocycle, each of which comprises one, two, three or four heteroatoms from the group consisting of O, N and S, and NA³A⁴, where two radicals R^{z} attached to the same carbon atom, together with the carbon atom to which they are attached, may also form a C₃-C₆-cycloalkyl ring; where A³, A⁴ are as defined below.

According to one embodiment, R^{z} is in each case independently halogen, cyano, nitro, cyanato (OCN), C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₃-C₈-haloalkynyloxy, C₃-C₈-cycloalkyl, C₃-C₈-halocycloalkyl, C₃-C₈-cycloalkenyl, C₃-C₈-halocycloalkenyl, C₆-C₈-cycloalkynyl, C₆-C₈-halocycloalkynyl, C₃-C₈-cycloalkoxy, C₃-C₈-cycloalkenyloxy, or NA³A⁴.

According to a further embodiment, R^{z} is in each case independently Cl, F, Br, cyano, C₁-C₄-haloalkyl, C₂-C₄-alkenyl, C₂-C₄-haloalkenyl, C₁-C₄-alkoxy, C₁-C₄-haloalkoxy, C₃-C₆-cycloalkyl or C₃-C₆-halocycloalkyl, in particular methyl, ethyl, trifluoromethyl, methoxy, ethoxy or cyclopropyl.

According to a further embodiment, at least one R^{z} is halogen, in particular Cl or F.

According to a further embodiment, at least one R^{z} is C₁-C₄-alkyl, in particular methyl or ethyl.

According to a further embodiment, at least one R^{z} is C₁-C₄-haloalkyl.

According to a further embodiment, two radicals R^{z} which are attached to the same carbon atom form, together with the carbon atom to which they are attached, a C₃-C₆-cycloalkyl ring.

R¹ in the compounds according to the invention is a three-, four-, five-, six-, seven-, eight-, nine- or ten-membered saturated or partially unsaturated heterocycle or a five-, six-, seven-, eight-, nine- or ten-membered aromatic heterocycle, where the heterocycle contains in each case one, two, three or four heteroatoms from the group consisting of O, N and S, where the heterocycle is unsubstituted or contains one, two, three, four or five independently selected substituents L.

According to one embodiment, the heterocycle in question is attached via carbon. According to a further embodiment, the heterocycle is attached via nitrogen, if present.

According to one embodiment of the invention, R¹ is a 5-, 6-, 7-, 8- or 9-membered aromatic heterocycle which contains 1, 2, 3 or 4 heteroatoms from the group consisting of O, N and S, where the heteroaromatic is unsubstituted or contains one, two, three, four or five independently selected substituents L.

According to a specific embodiment, the heteroaromatic is an unsubstituted or substituted five-membered heteroaromatic which contains one, two or three heteroatoms from the group consisting of O, N and S. In particular, the five-membered heteroaromatic contains one, two, three or four nitrogen atoms or one, two or three nitrogen atoms and/or one sulfur or oxygen atom. Examples of 5-membered heteroaromatics as R¹ are furyl, thienyl, pyrrolyl, pyrazolyl, imidazolyl, triazolyl (1,2,3-; 1,2,4-triazolyl), oxazolyl, isoxazolyl, 1,3,4-oxadiazolyl, thiazolyl, isothiazolyl and thiadiazolyl, in particular 2-furyl, 3-furyl, 2-thienyl, 3-thienyl, 1-pyrrolyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl, 1,2,4-triazol-1-yl.

According to a specific embodiment, the heteroaromatic is an unsubstituted or substituted six-membered heteroaromatic which contains one, two, three or four, preferably one, two or three, nitrogen atoms. Examples of 6-membered heteroaromatics as R¹ are pyridinyl, pyrimidinyl, pyrazinyl, pyridazinyl, 1,2,3-triazinyl, 1,2,4-triazinyl, 1,3,5-triazinyl, tetrazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl. According to a specific embodiment, R¹ is pyridinyl.

According to a further specific embodiment, the heteroaromatic is an unsubstituted or substituted nine- or ten-membered heteroaromatic which contains one, two, three or four nitrogen atoms. Examples of nine- and ten-membered heteroaromatics as R¹ are purinyl, pteridinyl, quinolinyl, isoquinolinyl and indolyl, in particular 1-indolyl, benzimidazolyl, benzoxazolyl, benzofuranyl, benzothiazolyl or benzotriazolyl.

According to a further embodiment of the invention, R¹ is a 5-, 6- or 7-membered saturated heterocycle which contains 1, 2, 3 or 4 heteroatoms from the group consisting of O, N and S, where the heterocycle is unsubstituted or contains one, two, three, four or five independently selected substituents L.

According to a specific embodiment, the heteroaromatic is an unsubstituted or substituted five-membered saturated heterocycle which contains one, two or three heteroatoms from the group consisting of O, N and S. In particular, the heterocycle contains, in addition to carbon ring members, one, two or three nitrogen atoms and/or one oxygen or sulfur atom or one or two oxygen and/or sulfur atoms. Examples of five-membered saturated heterocycles as R¹ are 2-tetrahydrofuranyl, 3-tetrahydrofuranyl, 2-tetrahydrothienyl, 3-tetrahydrothienyl, 2-pyrrolidinyl and 3-pyrrolidinyl.

According to a specific embodiment, the heteroaromatic is an unsubstituted or substituted six-membered heterocycle which contains one, two or three heteroatoms from the group consisting of O, N and S. In particular, the heterocycle contains, in addition to carbon ring members, one, two or three nitrogen atoms and/or one oxygen or sulfur atom or one or two oxygen and/or sulfur atoms. Examples of six-membered saturated heterocycles as R¹ are 2-morpholinyl, 3-morpholinyl, 1-piperidinyl, 2-piperidinyl, 3-piperidinyl, 4-piperidinyl, 1,3-dioxan-5-yl, 2-tetrahydropyranyl, 4-tetrahydropyranyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydro-triazin-2-yl and 1,2,4-hexahydrotriazin-3-yl.

According to a further embodiment of the invention, R¹ is a 5- or 6-membered partially unsaturated heterocycle which contains 1, 2, 3 or 4 heteroatoms from the group consisting of O, N and S, where the heterocycle is unsubstituted or contains one, two, three, four or five independently selected substituents L. Examples are 2H-pyranyl, in particular 2H-pyran-2-yl, and dihydrooxazin-3-yl:

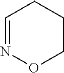

According to the present invention, R² is hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₄-C₁₀-alkadienyl, C₄-C₁₀-haloalkadienyl, C₃-C₁₀-cycloalkyl, C₃-C₁₀-halocycloalkyl, C₃-C₁₀-cycloalkenyl or C₃-C₁₀-halocycloalkenyl, where R² may contain one, two, three, four or five substituents L, as defined herein.

According to a preferred embodiment, R² is hydrogen.

According to a further embodiment, R² is C₁-C₁₀-alkyl, C₁-C₁₀-haloalkyl, phenyl-C₁-C₄-alkyl, C₂-C₁₀-alkenyl, C₂-C₁₀-haloalkenyl, C₂-C₁₀-alkynyl, C₃-C₁₀-haloalkynyl, C₄-C₁₀-alkadienyl, C₄-C₁₀-haloalkadienyl, C₃-C₁₀-cycloalkyl, C₃-C₁₀-halocycloalkyl, C₃-C₁₀-cycloalkenyl or C₃-C₁₀-halocycloalkenyl, in particular C₁-C₄-alkyl, C₂-C₄-alkenyl, C₃-C₄-alkynyl or phenyl-C₁-C₄-alkyl. Specific examples of R² are methyl, ethyl, n-propyl, isopropyl, n-butyl, tert-butyl, 2-vinyl, 3-allyl, 3-propargyl, 4-but-2-ynyl and benzyl.

According to the present invention, R³ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₄-C₁₀-alkadienyl, C₄-C₁₀-haloalkadienyl, C₃-C₁₀-cycloalkyl, C₃-C₁₀-halocycloalkyl, C₃-C₁₀-cycloalkenyl, C₃-C₁₀-halocycloalkenyl, carboxyl, formyl, Si(A⁵A⁶A⁷), C(O)Rⁿ, C(O)ORⁿ, C(S)ORⁿ, C(O)SRⁿ, C(S)SRⁿ, C(NR^{A})SRⁿ, C(S)Rⁿ, C(NRⁿ)N NA³A⁴, C(NRⁿ)R^{A}, C(NRⁿ)OR^{A}, C(O)NA³A⁴, C(S)NA³A⁴ or S(═O)ₙA¹; where
A¹ is hydrogen, hydroxyl, C₁-C₈-alkyl, C₁-C₈-haloalkyl, amino, C₁-C₈-alkylamino, di-C₁-C₈-alkylamino, phenyl, phenylamino or phenyl-C₁-C₈-alkylamino;
Rⁿ is C₁-C₈-alkyl, C₃-C₈-alkenyl, C₃-C₈-alkynyl, C₃-C₈-cycloalkyl, C₃-C₆-cycloalkenyl or phenyl;
R^{A} is C₁-C₈-alkyl, C₃-C₈-alkenyl, C₃-C₈-alkynyl, C₃-C₈-cycloalkyl, C₃-C₆-cycloalkenyl or phenyl;
A⁵, A⁶, A⁷ independently of one another are C₁-C₁₀-alkyl, C₃-C₈-alkenyl, C₃-C₈-alkynyl, C₃-C₆-cycloalkyl, C₃-C₆-cycloalkenyl or phenyl;
   where Rⁿ, R^{A}, A⁵, A⁶ and A⁷ are, unless indicated otherwise, independently of one another unsubstituted or substituted by one, two, three, four or five L, as defined above.

R³ may comprise one, two, three, four or five substituents L, as defined herein.

According to a preferred embodiment, R³ is hydrogen.

According to a further embodiment, R³ is C₁-C₁₀-alkyl, C₁-C₁₀-haloalkyl, phenyl-C₁-C₁₀-alkyl, C₂-C₁₀-alkenyl, C₂-C₁₀-haloalkenyl, C₂-C₁₀-alkynyl, C₃-C₁₀-haloalkynyl, C₄-C₁₀-alkadienyl, C₄-C₁₀-haloalkadienyl, C₃-C₁₀-cycloalkyl, C₃-C₁₀-halocycloalkyl, C₃-C₁₀-cycloalkenyl, C₃-C₁₀-halocycloalkenyl, carboxyl, formyl, Si(A⁵A⁶A⁷), C(O)Rⁿ, C(O)ORⁿ, C(S)ORⁿ, C(O)SRⁿ, C(S)SRⁿ, C(NR^{A})SRⁿ, C(S)Rⁿ, C(NRⁿ)N NA³A⁴, C(NRⁿ)R^{A}, C(NRⁿ)OR^{A}, C(O)NA³A⁴, C(S)NA³A⁴ or S(═O)ₙA¹, in particular C₁-C₄-alkyl, phenyl-C₁-C₄-alkyl, halophenyl-C₁-C₄-alkyl, C₂-C₄-alkenyl, C₃-C₄-alkynyl, tri-C₁-C₄-alkylsilyl, C(O)Rⁿ or S(═O)₂A¹, where
A¹ is hydroxyl, C₁-C₄-alkyl, phenyl or C₁-C₄-alkylphenyl;
Rⁿ is C₁-C₄-alkyl; carboxy-C₁-C₄-alkyl or carboxyphenyl;
R^{A} is C₁-C₄-alkyl, C₃-C₆-cycloalkyl or phenyl;
A⁵, A⁶, A⁷ independently of one another are C₁-C₄-alkyl or phenyl, where the phenyl ring is unsubstituted or substituted by one, two, three, four or five L, as described herein.

Specific examples of R³ are trimethylsilyl, methyl, ethyl, n-propyl, isopropyl, n-butyl, tert-butyl, 2-vinyl, 3-allyl, 3-propargyl, 4-but-2-ynyl, C(═O)CH₃, C(═O)CH₂CH₃, C(═O)CH₂CH₂CH₃, C(═O)(CH₂)₂COOH, C(═O)(CH₂)₃COOH, C(═O)(2-COOH—C₆H₄), SO₂OH, SO₂CH₃, SO₂C₆H₅, SO₂(4-methyl-C₆H₄), benzyl and 4-chlorobenzyl.

According to a specific embodiment, R³ is trimethylsilyl.

According to the present invention, R⁴ is hydrogen, C₁-C₁₀-alkyl, C₂-C₁₀-alkenyl, C₂-C₁₀-haloalkenyl, C₂-C₁₀-alkynyl, C₃-C₁₀-haloalkynyl, alkadienyl, C₄-C₁₀-haloalkadienyl, C₃-C₁₀-cycloalkyl, C₃-C₁₀-halocycloalkyl, C₃-C₁₀-cycloalkenyl or C₃-C₁₀-halocycloalkenyl, where R⁴ may contain one, two, three, four or five substituents L, as defined herein.

According to a preferred embodiment, R⁴ is hydrogen.

According to a further embodiment, R⁴ is C₁-C₁₀-alkyl, C₁-C₁₀-haloalkyl, phenyl-C₁-C₄-alkyl, C₂-C₁₀-alkenyl, C₂-C₁₀-haloalkenyl, C₂-C₁₀-alkynyl, C₃-C₁₀-halo-alkynyl, C₄-C₁₀-alkadienyl, C₄-C₁₀-haloalkadienyl, C₃-C₁₀-cycloalkyl, C₃-C₁₀-halo-cycloalkyl, C₃-C₁₀-cycloalkenyl or C₃-C₁₀-halocycloalkenyl, in particular C₁-C₆-alkyl, C₂-C₆-alkenyl, C₂-C₆-alkynyl or phenyl-C₁-C₄-alkyl. Specific examples of R⁴ are methyl, ethyl, n-propyl, isopropyl, n-butyl, tert-butyl and benzyl.

According to the present invention, R⁵ is C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, C₃-C₁₀-cycloalkenyl, C₃-C₁₀-halocycloalkenyl, phenyl, five-, six- or seven-membered heteroaryl which contains 1, 2, 3 or 4 heteroatoms from the group consisting of O, N and S or five-, six- or seven-membered saturated or partially unsaturated heterocyclyl which contains 1, 2, 3 or 4 heteroatoms from the group consisting of O, N and S, where R⁵ may contain one, two, three, four, five or six independently selected substituents L, as defined or as defined as being preferred herein.

According to one embodiment, R⁵ is C₁-C₁₀-alkyl, such as, in particular, CH₃, CH₂CH₃, CH₂CH₂CH₃, CH(CH₃)₂, CH₂(CH₂)₂CH₃, C(CH₃)₃, C(CH₃)₂CH₂CH₃, C(CH₃)₂CH(CH₃)₂, C(CH₃)₂C(CH₃)₃, CH₂(CH(CH₃)₂, CH₂C(CH₃)₃, CH(CH₃)C(CH₃)₃ or CH(CH₃)CH(CH₃)₂. According to one aspect of this embodiment, the alkyl group furthermore contains one or two substituents L, as defined or as defined as being preferred herein. Here, L is in particular selected from the group consisting of C₃-C₆-cycloalkyl, in particular cyclopropyl. A specific example of R⁵ is CH(CH₃)CH[CH₂CH₂]. (CH[CH₂CH₂] is the group

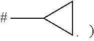

According to a further embodiment, R⁵ is C₁-C₁₀-haloalkyl, such as, for example, C(CH₃)(CH₂F)₂ or C(CH₃)₂F.

According to a further embodiment, R⁵ is C₂-C₁₀-alkenyl or C₂-C₁₀-haloalkenyl. A specific example of this embodiment is C(CH₃)₂CH═CH₂.

According to a further embodiment, R⁵ is a cyclic group, in particular C₃-C₁₀-cycloalkyl, where R⁵ is preferably selected from the group consisting of cyclopropyl, cyclopentyl, cyclohexyl, 1-methylcycloprop-1-yl, 1-chlorocycloprop-1-yl, 1-methylcyclopent-1-yl and 1-methylcyclohex-1-yl.

R⁵ is in each case unsubstituted or substituted by one, two, three, four, five or six independently selected substituents L, as defined or as defined as being preferred herein. According to one embodiment, R⁵ is not substituted any further by L. According to a further specific embodiment, L is selected from the group consisting of C₁-C₄-alkyl, C₁-C₄-haloalkyl, C₂-C₄-alkenyl, halogen (in particular Cl and/or F) and C₃-C₆-cycloalkyl. Specific examples are cyclopropyl, methylcyclopropyl, trifluoromethyl and difluoromethyl.

Independently, L has the meanings or preferred meanings mentioned above and in the claims for L. Unless indicated otherwise, L is preferably independently selected from the group consisting of halogen, cyano, nitro, cyanato (OCN), C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl, S-A¹, C(═O)A², C(═S)A², NASA; where A¹, A², A³, A⁴ are as defined below:
A¹ is hydrogen, hydroxyl, C₁-C₄-alkyl, C₁-C₄-haloalkyl;
A² is one of the groups mentioned for A₁ or C₁-C₄-alkoxy, C₁-C₄-haloalkoxy, C₃-C₆-cycloalkyl, C₃-C₆-halocycloalkyl, C₃-C₆-cycloalkoxy or C₃-C₆-halocycloalkoxy;
A³,A⁴ independently of one another are hydrogen, C₁-C₄-alkyl, C₁-C₄-haloalkyl;
where the aliphatic and/or alicyclic and/or aromatic groups of the radical definitions of L for their part may carry one, two, three or four identical or different groups R^{L}:
R^{L} is halogen, cyano, nitro, C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl, amino, C₁-C₈-alkylamino, di-C₁-C₈-alkylamino.

Furthermore preferably, L is independently selected from the group consisting of halogen, amino, C₁-C₄-alkyl, C₁-C₄-alkoxy, C₁-C₄-haloalkyl, C₁-C₄-haloalkoxy, C₁-C₄-alkylamino, C₁-C₄-dialkylamino, thio and C₁-C₄-alkylthio

Furthermore preferably, L is independently selected from the group consisting of halogen, C₁-C₄-alkyl, C₁-C₄-haloalkyl, C₁-C₄-alkoxy and C₁-C₄-haloalkoxy.

According to a further preferred embodiment, L is independently selected from the group consisting of F, Cl, Br, CH₃, C₂H₅, i-C₃H₇, t-C₄H₉, OCH₃, OC₂H₅, CF₃, CCl₃, CHF₂, CClF₂, OCF₃, OCHF₂ and SCF₃, in particular selected from the group consisting of F, Cl, CH₃, C₂H₅, OCH₃, OC₂H₅, CF₃, CHF₂, OCF₃, OCHF₂ and SCF₃. According to one aspect, L is independently selected from the group consisting of F, Cl, CH₃, OCH₃, CF₃, OCF₃ and OCHF₂. It may be preferred for L to be independently F or Cl.

According to a further embodiment, L is independently selected from the group consisting of F, Br, CH₃, C₂H₅, t-C₄H₉, OCH₃, OC₂H₅, CF₃, CCl₃, CHF₂, CClF₂, OCF₃, OCHF₂ and SCF₃.

According to yet a further embodiment, L is independently selected from the group consisting of F, Cl, Br, methyl and methoxy.

The meanings described above of the variables R¹, R², R³, R⁴, R⁵, X, Y, Z and L for compounds I apply correspondingly to the precursors of the compounds according to the invention.

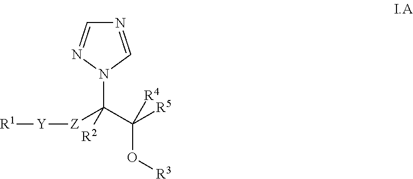

In particular with a view to their use, preference is given to the compounds I.A according to the invention compiled in Tables 1a bis 351a below. The groups mentioned for a substituent in the tables are furthermore per se, independently of the combination in which they are mentioned, a particularly preferred aspect of the substituent in question.

### Table 1a

Compounds I.A in which Z is CH₂CH₂CH₂, R², R³ and R⁴ are H, R⁵ is CH(CH₃)₂ and the combination of R¹ and Y corresponds in each case to one row of Table A (compounds I.A.1aA-1 to I.A.1aA-366)

### Table 2a

Compounds I.A in which Z is CH₂(CH₂)₂CH₂, R², R³ and R⁴ are H, R⁵ is CH(CH₃)₂ and the combination of R¹ and Y corresponds in each case to one row of Table A (compounds I.A.2aA-1 to I.A.2aA-366)

### Table 3a

Compounds I.A in which Z is CH₂(CH₂)₂CH₂, R², R³ and R⁴ are H, R⁵ is CH(CH₃)₂ and the combination of R¹ and Y corresponds in each case to one row of Table A (compounds I.A.3aA-1 to I.A.3aA-366)

### Table 4a

Compounds I.A in which Z is CH₂CH(CH₃)CH₂, R², R³ and R⁴ are H, R⁵ is CH(CH₃)₂ and the combination of R¹ and Y corresponds in each case to one row of Table A (compounds I.A.4aA-1 to I.A.4aA-366)

### Table 5a

Compounds I.A in which Z is CH₂CH₂CH(CH₃), R², R³ and R⁴ are H, R⁵ is CH(CH₃)₂ and the combination of R¹ and Y corresponds in each case to one row of Table A (compounds I.A.5aA-1 to I.A.5aA-366)

### Table 6a

Compounds I.A in which Z is CH(CH₃)CH₂CH₂, R², R³ and R⁴ are H, R⁵ is CH(CH₃)₂ and the combination of R¹ and Y corresponds in each case to one row of Table A (compounds I.A.6aA-1 to I.A.6aA-366)

### Table 7a

Compounds I.A in which Z is CH₂C(CH₃)₂CH₂, R², R³ and R⁴ are H, R⁵ is CH(CH₃)₂ and the combination of R¹ and Y corresponds in each case to one row of Table A (compounds I.A.7aA-1 to I.A.7aA-366)

### Table 8a

Compounds I.A in which Z is CH₂C(CH₂CH₂)CH₂, R², R³ and R⁴ are H, R⁵ is CH(CH₃)₂ and the combination of R¹ and Y corresponds in each case to one row of Table A (compounds I.A.8aA-1 to I.A.8aA-366)

### Table 9a

Compounds I.A in which Z is C(CH₂CH₂)CH₂CH₂, R², R³ and R⁴ are H, R⁵ is CH(CH₃)₂ and the combination of R¹ and Y corresponds in each case to one row of Table A (compounds I.A.9aA-1 to I.A.9aA-366)

### Table 10a

Compounds I.A in which Z is CH₂CH(CH₃)(CH₂)₂CH₂, R², R³ and R⁴ are H, R⁵ is CH(CH₃)₂ and the combination of R¹ and Y corresponds in each case to one row of Table A (compounds I.A.10aA-1 to I.A.10aA-366)

### Table 11a

Compounds I.A in which Z is C(CH₃)₂(CH₂)₃CH₂, R², R³ and R⁴ are H, R⁵ is CH(CH₃)₂ and the combination of R¹ and Y corresponds in each case to one row of Table A (compounds I.A.11aA-1 to I.A.11aA-366)

### Table 12a

Compounds I.A in which Z is C(CH₂CH₂)(CH₂)₃CH₂, R², R³ and R⁴ are H, R⁵ is CH(CH₃)₂ and the combination of R¹ and Y corresponds in each case to one row of Table A (compounds I.A.12aA-1 to I.A.12aA-366)

### Table 13a

Compounds I.A in which Z is CH₂CH₂CH(CH₃)CH₂CH₂, R², R³ and R⁴ are H, R⁵ is CH(CH₃)₂ and the combination of R¹ and Y corresponds in each case to one row of Table A (compounds I.A.13aA-1 to I.A.13aA-366)

### Table 14a

Compounds I.A in which Z is CH₂CH₂CH₂CH(CH₃)CH₂, R², R³ and R⁴ are H, R⁵ is CH(CH₃)₂ and the combination of R¹ and Y corresponds in each case to one row of Table A (compounds I.A.14aA-1 to I.A.14aA-366)

### Table 15a

Compounds I.A in which Z is CH₂(CH₂)₃CH(CH₃), R², R³ and R⁴ are H, R⁵ is CH(CH₃)₂ and the combination of R¹ and Y corresponds in each case to one row of Table A (compounds I.A.15aA-1 to I.A.15aA-366)

### Table 16a

Compounds I.A in which Z is (E) CH═CHCH₂, R², R³ and R⁴ are H, R⁵ is CH(CH₃)₂ and the combination of R¹ and Y corresponds in each case to one row of Table A (compounds I.A.16aA-1 to I.A.16aA-366)

### Table 17a

Compounds I.A in which Z is (E) C(CH₃)═CHCH₂, R², R³ and R⁴ are H, R⁵ is CH(CH₃)₂ and the combination of R¹ and Y corresponds in each case to one row of Table A (compounds I.A.17aA-1 to I.A.17aA-366)

### Table 18a

Compounds I.A in which Z is (E) C(CH₃)═C(CH₃)CH₂, R², R³ and R⁴ are H, R⁵ is CH(CH₃)₂ and the combination of R¹ and Y corresponds in each case to one row of Table A (compounds I.A.18aA-1 to I.A.18aA-366)

### Table 19a

Compounds I.A in which Z is (E) CH═C(CH₃)CH₂, R², R³ and R⁴ are H, R⁵ is CH(CH₃)₂ and the combination of R¹ and Y corresponds in each case to one row of Table A (compounds I.A.19aA-1 to I.A.19aA-366)

### Table 20a

Compounds I.A in which Z is (E) CH₂CH═CHCH₂, R², R³ and R⁴ are H, R⁵ is CH(CH₃)₂ and the combination of R¹ and Y corresponds in each case to one row of Table A (compounds I.A.20aA-1 to I.A.20aA-366)

### Table 21a

Compounds I.A in which Z is (E) CH₂C(CH₃)═CHCH₂, R², R³ and R⁴ are H, R⁵ is CH(CH₃)₂ and the combination of R¹ and Y corresponds in each case to one row of Table A (compounds I.A.21aA-1 to I.A.21aA-366)

### Table 22a

Compounds I.A in which Z is (E) CH₂CH═C(CH₃)CH₂, R², R³ and R⁴ are H, R⁵ is CH(CH₃)₂ and the combination of R¹ and Y corresponds in each case to one row of Table A (compounds I.A.22aA-1 to I.A.22aA-366)

### Table 23a

Compounds I.A in which Z is (E) CH₂C(CH₃)═C(CH₃)CH₂, R², R³ and R⁴ are H, R⁵ is CH(CH₃)₂ and the combination of R¹ and Y corresponds in each case to one row of Table A (compounds I.A.23aA-1 to I.A.23aA-366)

### Table 24a

Compounds I.A in which Z is (E) C(CH₃)═C(CH₃)(CH₂)₂CH₂, R², R³ and R⁴ are H, R⁵ is CH(CH₃)₂ and the combination of R¹ and Y corresponds in each case to one row of Table A (compounds I.A.24aA-1 to I.A.24aA-366)

### Table 25a

Compounds I.A in which Z is (E) C(CH₃)═CH(CH₂)₂CH₂, R², R³ and R⁴ are H, R⁵ is CH(CH₃)₂ and the combination of R¹ and Y corresponds in each case to one row of Table A (compounds I.A.25aA-1 to I.A.25aA-366)

### Table 26a

Compounds I.A in which Z is (E) CH═C(CH₃)(CH₂)₂CH₂, R², R³ and R⁴ are H, R⁵ is CH(CH₃)₂ and the combination of R¹ and Y corresponds in each case to one row of Table A (compounds I.A.26aA-1 to I.A.26aA-366)

### Table 27a

Compounds I.A in which Z is CH₂C═CCH₂, R², R³ and R⁴ are H, R⁵ is CH(CH₃)₂ and the combination of R¹ and Y corresponds in each case to one row of Table A (compounds I.A.27aA-1 to I.A.27aA-366)

### Table 28a

Compounds I.A in which Z is CH₂CH₂CH₂, R², R³ and R⁴ are H, R⁵ is CH₂CH₂CH₃ and the combination of R¹ and Y corresponds in each case to one row of Table A (compounds I.A.28aA-1 to I.A.28aA-366)

### Table 29a

Compounds I.A in which Z is CH₂(CH₂)₂CH₂, R², R³ and R⁴ are H, R⁵ is CH₂CH₂CH₃ and the combination of R¹ and Y corresponds in each case to one row of Table A (compounds I.A.29aA-1 to I.A.29aA-366)

### Table 30a

Compounds I.A in which Z is CH₂(CH₂)₃CH₂, R², R³ and R⁴ are H, R⁵ is CH₂CH₂CH₃ and the combination of R¹ and Y corresponds in each case to one row of Table A (compounds I.A.30aA-1 to I.A.30aA-366)

### Table 31a

Compounds I.A in which Z is CH₂CH(CH₃)CH₂, R², R³ and R⁴ are H, R⁵ is CH₂CH₂CH₃ and the combination of R¹ and Y corresponds in each case to one row of Table A (compounds I.A.31aA-1 to I.A.31aA-366)

### Table 32a

Compounds I.A in which Z is CH₂CH₂CH(CH₃), R², R³ and R⁴ are H, R⁵ is CH₂CH₂CH₃ and the combination of R¹ and Y corresponds in each case to one row of Table A (compounds I.A.32aA-1 to I.A.32aA-366)

### Table 33a

Compounds I.A in which Z is CH(CH₃)CH₂CH₂, R², R³ and R⁴ are H, R⁵ is CH₂CH₂CH₃ and the combination of R¹ and Y corresponds in each case to one row of Table A (compounds I.A.33aA-1 to I.A.33aA-366)

### Table 34a

Compounds I.A in which Z is CH₂C(CH₃)₂CH₂, R², R³ and R⁴ are H, R⁵ is CH₂CH₂CH₃ and the combination of R¹ and Y corresponds in each case to one row of Table A (compounds I.A.34aA-1 to I.A.34aA-366)

### Table 35a

Compounds I.A in which Z is CH₂C(CH₂CH₂)CH₂, R², R³ and R⁴ are H, R⁵ is CH₂CH₂CH₃ and the combination of R¹ and Y corresponds in each case to one row of Table A (compounds I.A.35aA-1 to I.A.35aA-366)

### Table 36a

Compounds I.A in which Z is C(CH₂CH₂)CH₂CH₂, R², R³ and R⁴ are H, R⁵ is CH₂CH₂CH₃ and the combination of R¹ and Y corresponds in each case to one row of Table A (compounds I.A.36aA-1 to I.A.36aA-366)

### Table 37a

Compounds I.A in which Z is CH₂CH(CH₃)(CH₂)₂CH₂, R², R³ and R⁴ are H, R⁵ is CH₂CH₂CH₃ and the combination of R¹ and Y corresponds in each case to one row of Table A (compounds I.A.37aA-1 to I.A.37aA-366)

### Table 38a

Compounds I.A in which Z is C(CH₃)₂(CH₂)₃CH₂, R², R³ and R⁴ are H, R⁵ is CH₂CH₂CH₃ and the combination of R¹ and Y corresponds in each case to one row of Table A (compounds I.A.38aA-1 to I.A.38aA-366)

### Table 39a

Compounds I.A in which Z is C(CH₂CH₂)(CH₂)₃CH₂, R², R³ and R⁴ are H, R⁵ is CH₂CH₂CH₃ and the combination of R¹ and Y corresponds in each case to one row of Table A (compounds I.A.39aA-1 to I.A.39aA-366)

### Table 40a

Compounds I.A in which Z is CH₂CH₂CH(CH₃)CH₂CH₂, R², R³ and R⁴ are H, R⁵ is CH₂CH₂CH₃ and the combination of R¹ and Y corresponds in each case to one row of Table A (compounds I.A.40aA-1 to I.A.40aA-366)

### Table 41a

Compounds I.A in which Z is CH₂CH₂CH₂CH(CH₃)CH₂, R², R³ and R⁴ are H, R⁵ is CH₂CH₂CH₃ and the combination of R¹ and Y corresponds in each case to one row of Table A (compounds I.A.41aA-1 to I.A.41aA-366)

### Table 42a

Compounds I.A in which Z is CH₂(CH₂)₃CH(CH₃), R², R³ and R⁴ are H, R⁵ is CH₂CH₂CH₃ and the combination of R¹ and Y corresponds in each case to one row of Table A (compounds I.A.42aA-1 to I.A.42aA-366)

### Table 43a

Compounds I.A in which Z is (E) CH═CHCH₂, R², R³ and R⁴ are H, R⁵ is CH₂CH₂CH₃ and the combination of R¹ and Y corresponds in each case to one row of Table A (compounds I.A.43aA-1 to I.A.43aA-366)

### Table 44a

Compounds I.A in which Z is (E) C(CH₃)═CHCH₂, R², R³ and R⁴ are H, R⁵ is CH₂CH₂CH₃ and the combination of R¹ and Y corresponds in each case to one row of Table A (compounds I.A.44aA-1 to I.A.44aA-366)

### Table 45a

Compounds I.A in which Z is (E) C(CH₃)═C(CH₃)CH₂, R², R³ and R⁴ are H, R⁵ is CH₂CH₂CH₃ and the combination of R¹ and Y corresponds in each case to one row of Table A (compounds I.A.45aA-1 to I.A.45aA-366)

### Table 46a

Compounds I.A in which Z is (E) CH═C(CH₃)CH₂, R², R³ and R⁴ are H, R⁵ is CH₂CH₂CH₃ and the combination of R¹ and Y corresponds in each case to one row of Table A (compounds I.A.46aA-1 to I.A.46aA-366)

### Table 47a

Compounds I.A in which Z is (E) CH₂CH═CHCH₂, R², R³ and R⁴ are H, R⁵ is CH₂CH₂CH₃ and the combination of R¹ and Y corresponds in each case to one row of Table A (compounds I.A.47aA-1 to I.A.47aA-366)

### Table 48a

Compounds I.A in which Z is (E) CH₂C(CH₃)═CHCH₂, R², R³ and R⁴ are H, R⁵ is CH₂CH₂CH₃ and the combination of R¹ and Y corresponds in each case to one row of Table A (compounds I.A.48aA-1 to I.A.48aA-366)

### Table 49a

Compounds I.A in which Z is (E) CH₂CH═C(CH₃)CH₂, R², R³ and R⁴ are H, R⁵ is CH₂CH₂CH₃ and the combination of R¹ and Y corresponds in each case to one row of Table A (compounds I.A.49aA-1 to I.A.49aA-366)

### Table 50a

Compounds I.A in which Z is (E) CH₂C(CH₃)═C(CH₃)CH₂, R², R³ and R⁴ are H, R⁵ is CH₂CH₂CH₃ and the combination of R¹ and Y corresponds in each case to one row of Table A (compounds I.A.50aA-1 to I.A.50aA-366)

### Table 51a

Compounds I.A in which Z is (E) C(CH₃)═C(CH₃)(CH₂)₂CH₂, R², R³ and R⁴ are H, R⁵ is CH₂CH₂CH₃ and the combination of R¹ and Y corresponds in each case to one row of Table A (compounds I.A.51aA-1 to I.A.51aA-366)

### Table 52a

Compounds I.A in which Z is (E) C(CH₃)═CH(CH₂)₂CH₂, R², R³ and R⁴ are H, R⁵ is CH₂CH₂CH₃ and the combination of R¹ and Y corresponds in each case to one row of Table A (compounds I.A.52aA-1 to I.A.52aA-366)

### Table 53a

Compounds I.A in which Z is (E) CH═C(CH₃)(CH₂)₂CH₂, R², R³ and R⁴ are H, R⁵ is CH₂CH₂CH₃ and the combination of R¹ and Y corresponds in each case to one row of Table A (compounds I.A.53aA-1 to I.A.53aA-366)

### Table 54a

Compounds I.A in which Z is CH₂C≡CCH₂, R², R³ and R⁴ are H, R⁵ is CH₂CH₂CH₃ and the combination of R¹ and Y corresponds in each case to one row of Table A (compounds I.A.54aA-1 to I.A.54aA-366)

### Table 55a

Compounds I.A in which Z is CH₂CH₂CH₂, R², R³ and R⁴ are H, R⁵ is CH₂(CH₂)₂CH₃ and the combination of R¹ and Y corresponds in each case to one row of Table A (compounds I.A.55aA-1 to I.A.55aA-366)

### Table 56a

Compounds I.A in which Z is CH₂(CH₂)₂CH₂, R², R³ and R⁴ are H, R⁵ is CH₂(CH₂)₂CH₃ and the combination of R¹ and Y corresponds in each case to one row of Table A (compounds I.A.56aA-1 to I.A.56aA-366)

### Table 57a

Compounds I.A in which Z is CH₂(CH₂)₃CH₂, R², R³ and R⁴ are H, R⁵ is CH₂(CH₂)₂CH₃ and the combination of R¹ and Y corresponds in each case to one row of Table A (compounds I.A.57aA-1 to I.A.57aA-366)

### Table 58a

Compounds I.A in which Z is CH₂CH(CH₃)CH₂, R², R³ and R⁴ are H, R⁵ is CH₂(CH₂)₂CH₃ and the combination of R¹ and Y corresponds in each case to one row of Table A (compounds I.A.58aA-1 to I.A.58aA-366)

### Table 59a

Compounds I.A in which Z is CH₂CH₂CH(CH₃), R², R³ and R⁴ are H, R⁵ is CH₂(CH₂)₂CH₃ and the combination of R¹ and Y corresponds in each case to one row of Table A (compounds I.A.59aA-1 to I.A.59aA-366)

### Table 60a

Compounds I.A in which Z is CH(CH₃)CH₂CH₂, R², R³ and R⁴ are H, R⁵ is CH₂(CH₂)₂CH₃ and the combination of R¹ and Y corresponds in each case to one row of Table A (compounds I.A.60aA-1 to I.A.60aA-366)

### Table 61a

Compounds I.A in which Z is CH₂C(CH₃)₂CH₂, R², R³ and R⁴ are H, R⁵ is CH₂(CH₂)₂CH₃ and the combination of R¹ and Y corresponds in each case to one row of Table A (compounds I.A.61aA-1 to I.A.61aA-366)

### Table 62a

Compounds I.A in which Z is CH₂C(CH₂CH₂)CH₂, R², R³ and R⁴ are H, R⁵ is CH₂(CH₂)₂CH₃ and the combination of R¹ and Y corresponds in each case to one row of Table A (compounds I.A.62aA-1 to I.A.62aA-366)

### Table 63a

Compounds I.A in which Z is C(CH₂CH₂)CH₂CH₂, R², R³ and R⁴ are H, R⁵ is CH₂(CH₂)₂CH₃ and the combination of R¹ and Y corresponds in each case to one row of Table A (compounds I.A.63aA-1 to I.A.63aA-366)

### Table 64a

Compounds I.A in which Z is CH₂CH(CH₃)(CH₂)₂CH₂, R², R³ and R⁴ are H, R⁵ is CH₂(CH₂)₂CH₃ and the combination of R¹ and Y corresponds in each case to one row of Table A (compounds I.A.64aA-1 to I.A.64aA-366)

### Table 65a

Compounds I.A in which Z is C(CH₃)₂(CH₂)₃CH₂, R², R³ and R⁴ are H, R⁵ is CH₂(CH₂)₂CH₃ and the combination of R¹ and Y corresponds in each case to one row of Table A (compounds I.A.65aA-1 to I.A.65aA-366)

### Table 66a

Compounds I.A in which Z is C(CH₂CH₂)(CH₂)₃CH₂, R², R³ and R⁴ are H, R⁵ is CH₂(CH₂)₂CH₃ and the combination of R¹ and Y corresponds in each case to one row of Table A (compounds I.A.66aA-1 to I.A.66aA-366)

### Table 67a

Compounds I.A in which Z is CH₂CH₂CH(CH₃)CH₂CH₂, R², R³ and R⁴ are H, R⁵ is CH₂(CH₂)₂CH₃ and the combination of R¹ and Y corresponds in each case to one row of Table A (compounds I.A.67aA-1 to I.A.67aA-366)

### Table 68a

Compounds I.A in which Z is CH₂CH₂CH₂CH(CH₃)CH₂, R², R³ and R⁴ are H, R⁵ is CH₂(CH₂)₂CH₃ and the combination of R¹ and Y corresponds in each case to one row of Table A (compounds I.A.68aA-1 to I.A.68aA-366)

### Table 69a

Compounds I.A in which Z is CH₂(CH₂)₃CH(CH₃), R², R³ and R⁴ are H, R⁵ is CH₂(CH₂)₂CH₃ and the combination of R¹ and Y corresponds in each case to one row of Table A (compounds I.A.69aA-1 to I.A.69aA-366)

### Table 70a

Compounds I.A in which Z is (E) CH═CHCH₂, R², R³ and R⁴ are H, R⁵ is CH₂(CH₂)₂CH₃ and the combination of R¹ and Y corresponds in each case to one row of Table A (compounds I.A.70aA-1 to I.A.70aA-366)

### Table 71a

Compounds I.A in which Z is (E) C(CH₃)═CHCH₂, R², R³ and R⁴ are H, R⁵ is CH₂(CH₂)₂CH₃ and the combination of R¹ and Y corresponds in each case to one row of Table A (compounds I.A.71aA-1 to I.A.71aA-366)

### Table 72a

Compounds I.A in which Z is (E) C(CH₃)═C(CH₃)CH₂, R², R³ and R⁴ are H, R⁵ is CH₂(CH₂)₂CH₃ and the combination of R¹ and Y corresponds in each case to one row of Table A (compounds I.A.72aA-1 to I.A.72aA-366)

### Table 73a

Compounds I.A in which Z is (E) CH═C(CH₃)CH₂, R², R³ and R⁴ are H, R⁵ is CH₂(CH₂)₂CH₃ and the combination of R¹ and Y corresponds in each case to one row of Table A (compounds I.A.73aA-1 to I.A.73aA-366)

### Table 74a

Compounds I.A in which Z is (E) CH₂CH═CHCH₂, R², R³ and R⁴ are H, R⁵ is CH₂(CH₂)₂CH₃ and the combination of R¹ and Y corresponds in each case to one row of Table A (compounds I.A.74aA-1 to I.A.74aA-366)

### Table 75a

Compounds I.A in which Z is (E) CH₂C(CH₃)═CHCH₂, R², R³ and R⁴ are H, R⁵ is CH₂(CH₂)₂CH₃ and the combination of R¹ and Y corresponds in each case to one row of Table A (compounds I.A.75aA-1 to I.A.75aA-366)

### Table 76a

Compounds I.A in which Z is (E) CH₂CH═C(CH₃)CH₂, R², R³ and R⁴ are H, R⁵ is CH₂(CH₂)₂CH₃ and the combination of R¹ and Y corresponds in each case to one row of Table A (compounds I.A.76aA-1 to I.A.76aA-366)

### Table 77a

Compounds I.A in which Z is (E) CH₂C(CH₃)═C(CH₃)CH₂, R², R³ and R⁴ are H, R⁵ is CH₂(CH₂)₂CH₃ and the combination of R¹ and Y corresponds in each case to one row of Table A (compounds I.A.77aA-1 to I.A.77aA-366)

### Table 78a

Compounds I.A in which Z is (E) C(CH₃)═C(CH₃)(CH₂)₂CH₂, R², R³ and R⁴ are H, R⁵ is CH₂(CH₂)₂CH₃ and the combination of R¹ and Y corresponds in each case to one row of Table A (compounds I.A.78aA-1 to I.A.78aA-366)

### Table 79a

Compounds I.A in which Z is (E) C(CH₃)═CH(CH₂)₂CH₂, R², R³ and R⁴ are H, R⁵ is CH₂(CH₂)₂CH₃ and the combination of R¹ and Y corresponds in each case to one row of Table A (compounds I.A.79aA-1 to I.A.79aA-366)

### Table 80a

Compounds I.A in which Z is (E) CH═C(CH₃)(CH₂)₂CH₂, R², R³ and R⁴ are H, R⁵ is CH₂(CH₂)₂CH₃ and the combination of R¹ and Y corresponds in each case to one row of Table A (compounds I.A.80aA-1 to I.A.80aA-366)

### Table 81a

Compounds I.A in which Z is CH₂C≡CCH₂, R², R³ and R⁴ are H, R⁵ is CH₂(CH₂)₂CH₃ and the combination of R¹ and Y corresponds in each case to one row of Table A (compounds I.A.81aA-1 to I.A.81aA-366)

### Table 82a

Compounds I.A in which Z is CH₂CH₂CH₂, R², R³ and R⁴ are H, R⁵ is C(CH₃)₂CH₂CH₃ and the combination of R¹ and Y corresponds in each case to one row of Table A (compounds I.A.82aA-1 to I.A.82aA-366)

### Table 83a

Compounds I.A in which Z is CH₂(CH₂)₂CH₂, R², R³ and R⁴ are H, R⁵ is C(CH₃)₂CH₂CH₃ and the combination of R¹ and Y corresponds in each case to one row of Table A (compounds I.A.83aA-1 to I.A.83aA-366)

### Table 84a

Compounds I.A in which Z is CH₂(CH₂)₃CH₂, R², R³ and R⁴ are H, R⁵ is C(CH₃)₂CH₂CH₃ and the combination of R¹ and Y corresponds in each case to one row of Table A (compounds I.A.84aA-1 to I.A.84aA-366)

### Table 85a

Compounds I.A in which Z is CH₂CH(CH₃)CH₂, R², R³ and R⁴ are H, R⁵ is C(CH₃)₂CH₂CH₃ and the combination of R¹ and Y corresponds in each case to one row of Table A (compounds I.A.85aA-1 to I.A.85aA-366)

### Table 86a

Compounds I.A in which Z is CH₂CH₂CH(CH₃), R², R³ and R⁴ are H, R⁵ is C(CH₃)₂CH₂CH₃ and the combination of R¹ and Y corresponds in each case to one row of Table A (compounds I.A.86aA-1 to I.A.86aA-366)

### Table 87a

Compounds I.A in which Z is CH(CH₃)CH₂CH₂, R², R³ and R⁴ are H, R⁵ is C(CH₃)₂CH₂CH₃ and the combination of R¹ and Y corresponds in each case to one row of Table A (compounds I.A.87aA-1 to I.A.87aA-366)

### Table 88a

Compounds I.A in which Z is CH₂C(CH₃)₂CH₂, R², R³ and R⁴ are H, R⁵ is C(CH₃)₂CH₂CH₃ and the combination of R¹ and Y corresponds in each case to one row of Table A (compounds I.A.88aA-1 to I.A.88aA-366)

### Table 89a

Compounds I.A in which Z is CH₂C(CH₂CH₂)CH₂, R², R³ and R⁴ are H, R⁵ is C(CH₃)₂CH₂CH₃ and the combination of R¹ and Y corresponds in each case to one row of Table A (compounds I.A.89aA-1 to I.A.89aA-366)

### Table 90a

Compounds I.A in which Z is C(CH₂CH₂)CH₂CH₂, R², R³ and R⁴ are H, R⁵ is C(CH₃)₂CH₂CH₃ and the combination of R¹ and Y corresponds in each case to one row of Table A (compounds I.A.90aA-1 to I.A.90aA-366)

### Table 91a

Compounds I.A in which Z is CH₂CH(CH₃)(CH₂)₂CH₂, R², R³ and R⁴ are H, R⁵ is C(CH₃)₂CH₂CH₃ and the combination of R¹ and Y corresponds in each case to one row of Table A (compounds I.A.91aA-1 to I.A.91aA-366)

### Table 92a

Compounds I.A in which Z is C(CH₃)₂(CH₂)₃CH₂, R², R³ and R⁴ are H, R⁵ is C(CH₃)₂CH₂CH₃ and the combination of R¹ and Y corresponds in each case to one row of Table A (compounds I.A.92aA-1 to I.A.92aA-366)

### Table 93a

Compounds I.A in which Z is C(CH₂CH₂)(CH₂)₃CH₂, R², R³ and R⁴ are H, R⁵ is C(CH₃)₂CH₂CH₃ and the combination of R¹ and Y corresponds in each case to one row of Table A (compounds I.A.93aA-1 to I.A.93aA-366)

### Table 94a

Compounds I.A in which Z is CH₂CH₂CH(CH₃)CH₂CH₂, R², R³ and R⁴ are H, R⁵ is C(CH₃)₂CH₂CH₃ and the combination of R¹ and Y corresponds in each case to one row of Table A (compounds I.A.94aA-1 to I.A.94aA-366)

### Table 95a

Compounds I.A in which Z is CH₂CH₂CH₂CH(CH₃)CH₂, R², R³ and R⁴ are H, R⁵ is C(CH₃)₂CH₂CH₃ and the combination of R¹ and Y corresponds in each case to one row of Table A (compounds I.A.95aA-1 to I.A.95aA-366)

### Table 96a

Compounds I.A in which Z is CH₂(CH₂)₃CH(CH₃), R², R³ and R⁴ are H, R⁵ is C(CH₃)₂CH₂CH₃ and the combination of R¹ and Y corresponds in each case to one row of Table A (compounds I.A.96aA-1 to I.A.96aA-366)

### Table 97a

Compounds I.A in which Z is (E) CH═CHCH₂, R², R³ and R⁴ are H, R⁵ is C(CH₃)₂CH₂CH₃ and the combination of R¹ and Y corresponds in each case to one row of Table A (compounds I.A.97aA-1 to I.A.97aA-366)

### Table 98a

Compounds I.A in which Z is (E) C(CH₃)═CHCH₂, R², R³ and R⁴ are H, R⁵ is C(CH₃)₂CH₂CH₃ and the combination of R¹ and Y corresponds in each case to one row of Table A (compounds I.A.98aA-1 to I.A.98aA-366)

### Table 99a

Compounds I.A in which Z is (E) C(CH₃)═C(CH₃)CH₂, R², R³ and R⁴ are H, R⁵ is C(CH₃)₂CH₂CH₃ and the combination of R¹ and Y corresponds in each case to one row of Table A (compounds I.A.99aA-1 to I.A.99aA-366)

### Table 100a

Compounds I.A in which Z is (E) CH═C(CH₃)CH₂, R², R³ and R⁴ are H, R⁵ is C(CH₃)₂CH₂CH₃ and the combination of R¹ and Y corresponds in each case to one row of Table A (compounds I.A.100aA-1 to I.A.100aA-366)

### Table 101a

Compounds I.A in which Z is (E) CH₂CH═CHCH₂, R², R³ and R⁴ are H, R⁵ is C(CH₃)₂CH₂CH₃ and the combination of R¹ and Y corresponds in each case to one row of Table A (compounds I.A.101aA-1 to I.A.101aA-366)

### Table 102a

Compounds I.A in which Z is (E) CH₂C(CH₃)═CHCH₂, R², R³ and R⁴ are H, R⁵ is C(CH₃)₂CH₂CH₃ and the combination of R¹ and Y corresponds in each case to one row of Table A (compounds I.A.102aA-1 to I.A.102aA-366)

### Table 103a

Compounds I.A in which Z is (E) CH₂CH═C(CH₃)CH₂, R², R³ and R⁴ are H, R⁵ is C(CH₃)₂CH₂CH₃ and the combination of R¹ and Y corresponds in each case to one row of Table A (compounds I.A.103aA-1 to I.A.103aA-366)

### Table 104a

Compounds I.A in which Z is (E) CH₂C(CH₃)═C(CH₃)CH₂, R², R³ and R⁴ are H, R⁵ is C(CH₃)₂CH₂CH₃ and the combination of R¹ and Y corresponds in each case to one row of Table A (compounds I.A.104aA-1 to I.A.104aA-366)

### Table 105a

Compounds I.A in which Z is (E) C(CH₃)═C(CH₃)(CH₂)₂CH₂, R², R³ and R⁴ are H, R⁵ is C(CH₃)₂CH₂CH₃ and the combination of R¹ and Y corresponds in each case to one row of Table A (compounds I.A.105aA-1 to I.A.105aA-366)

### Table 106a

Compounds I.A in which Z is (E) C(CH₃)═CH(CH₂)₂CH₂, R², R³ and R⁴ are H, R⁵ is C(CH₃)₂CH₂CH₃ and the combination of R¹ and Y corresponds in each case to one row of Table A (compounds I.A.106aA-1 to I.A.106aA-366)

### Table 107a

Compounds I.A in which Z is (E) CH═C(CH₃)(CH₂)₂CH₂, R², R³ and R⁴ are H, R⁵ is C(CH₃)₂CH₂CH₃ and the combination of R¹ and Y corresponds in each case to one row of Table A (compounds I.A.107aA-1 to I.A.107aA-366)

### Table 108a

Compounds I.A in which Z is CH₂CCCH₂, R², R³ and R⁴ are H, R⁵ is C(CH₃)₂CH₂CH₃ and the combination of R¹ and Y corresponds in each case to one row of Table A (compounds I.A.108aA-1 to I.A.108aA-366)

### Table 109a

Compounds I.A in which Z is CH₂CH₂CH₂, R², R³ and R⁴ are H, R⁵ is C(CH₃)(CH₂CH₃)₂ and the combination of R¹ and Y corresponds in each case to one row of Table A (compounds I.A.109aA-1 to I.A.109aA-366)

### Table 110a

Compounds I.A in which Z is CH₂(CH₂)₂CH₂, R², R³ and R⁴ are H, R⁵ is C(CH₃)(CH₂CH₃)₂ and the combination of R¹ and Y corresponds in each case to one row of Table A (compounds I.A.110aA-1 to I.A.110aA-366)

### Table 111a

Compounds I.A in which Z is CH₂(CH₂)₃CH₂, R², R³ and R⁴ are H, R⁵ is C(CH₃)(CH₂CH₃)₂ and the combination of R¹ and Y corresponds in each case to one row of Table A (compounds I.A.111aA-1 to I.A.111aA-366)

### Table 112a

Compounds I.A in which Z is CH₂CH(CH₃)CH₂, R², R³ and R⁴ are H, R⁵ is C(CH₃)(CH₂CH₃)₂ and the combination of R¹ and Y corresponds in each case to one row of Table A (compounds I.A.112aA-1 to I.A.112aA-366)

### Table 113a

Compounds I.A in which Z is CH₂CH₂CH(CH₃), R², R³ and R⁴ are H, R⁵ is C(CH₃)(CH₂CH₃)₂ and the combination of R¹ and Y corresponds in each case to one row of Table A (compounds I.A.113aA-1 to I.A.113aA-366)

### Table 114a

Compounds I.A in which Z is CH(CH₃)CH₂CH₂, R², R³ and R⁴ are H, R⁵ is C(CH₃)(CH₂CH₃)₂ and the combination of R¹ and Y corresponds in each case to one row of Table A (compounds I.A.114aA-1 to I.A.114aA-366)

### Table 115a

Compounds I.A in which Z is CH₂C(CH₃)₂CH₂, R², R³ and R⁴ are H, R⁵ is C(CH₃)(CH₂CH₃)₂ and the combination of R¹ and Y corresponds in each case to one row of Table A (compounds I.A.115aA-1 to I.A.115aA-366)

### Table 116a

Compounds I.A in which Z is CH₂C(CH₂CH₂)CH₂, R², R³ and R⁴ are H, R⁵ is C(CH₃)(CH₂CH₃)₂ and the combination of R¹ and Y corresponds in each case to one row of Table A (compounds I.A.116aA-1 to I.A.116aA-366)

### Table 117a

Compounds I.A in which Z is C(CH₂CH₂)CH₂CH₂, R², R³ and R⁴ are H, R⁵ is C(CH₃)(CH₂CH₃)₂ and the combination of R¹ and Y corresponds in each case to one row of Table A (compounds I.A.117aA-1 to I.A.117aA-366)

### Table 118a

Compounds I.A in which Z is CH₂CH(CH₃)(CH₂)₂CH₂, R², R³ and R⁴ are H, R⁵ is C(CH₃)(CH₂CH₃)₂ and the combination of R¹ and Y corresponds in each case to one row of Table A (compounds I.A.118aA-1 to I.A.118aA-366)

### Table 119a

Compounds I.A in which Z is C(CH₃)₂(CH₂)₃CH₂, R², R³ and R⁴ are H, R⁵ is C(CH₃)(CH₂CH₃)₂ and the combination of R¹ and Y corresponds in each case to one row of Table A (compounds I.A.119aA-1 to I.A.119aA-366)

### Table 120a

Compounds I.A in which Z is C(CH₂CH₂)(CH₂)₃CH₂, R², R³ and R⁴ are H, R⁵ is C(CH₃)(CH₂CH₃)₂ and the combination of R¹ and Y corresponds in each case to one row of Table A (compounds I.A.120aA-1 to I.A.120aA-366)

### Table 121a

Compounds I.A in which Z is CH₂CH₂CH(CH₃)CH₂CH₂, R², R³ and R⁴ are H, R⁵ is C(CH₃)(CH₂CH₃)₂ and the combination of R¹ and Y corresponds in each case to one row of Table A (compounds I.A.121aA-1 to I.A.121aA-366)

### Table 122a

Compounds I.A in which Z is CH₂CH₂CH₂CH(CH₃)CH₂, R², R³ and R⁴ are H, R⁵ is C(CH₃)(CH₂CH₃)₂ and the combination of R¹ and Y corresponds in each case to one row of Table A (compounds I.A.122aA-1 to I.A.122aA-366)

### Table 123a

Compounds I.A in which Z is CH₂(CH₂)₃CH(CH₃), R², R³ and R⁴ are H, R⁵ is C(CH₃)(CH₂CH₃)₂ and the combination of R¹ and Y corresponds in each case to one row of Table A (compounds I.A.123aA-1 to I.A.123aA-366)

### Table 124a

Compounds I.A in which Z is (E) CH═CHCH₂, R², R³ and R⁴ are H, R⁵ is C(CH₃)(CH₂CH₃)₂ and the combination of R¹ and Y corresponds in each case to one row of Table A (compounds I.A.124aA-1 to I.A.124aA-366)

### Table 125a

Compounds I.A in which Z is (E) C(CH₃)═CHCH₂, R², R³ and R⁴ are H, R⁵ is C(CH₃)(CH₂CH₃)₂ and the combination of R¹ and Y corresponds in each case to one row of Table A (compounds I.A.125aA-1 to I.A.125aA-366)

### Table 126a

Compounds I.A in which Z is (E) C(CH₃)═C(CH₃)CH₂, R², R³ and R⁴ are H, R⁵ is C(CH₃)(CH₂CH₃)₂ and the combination of R¹ and Y corresponds in each case to one row of Table A (compounds I.A.126aA-1 to I.A.126aA-366)

### Table 127a

Compounds I.A in which Z is (E) CH═C(CH₃)CH₂, R², R³ and R⁴ are H, R⁵ is C(CH₃)(CH₂CH₃)₂ and the combination of R¹ and Y corresponds in each case to one row of Table A (compounds I.A.127aA-1 to I.A.127aA-366)

### Table 128a

Compounds I.A in which Z is (E) CH₂CH═CHCH₂, R², R³ and R⁴ are H, R⁵ is C(CH₃)(CH₂CH₃)₂ and the combination of R¹ and Y corresponds in each case to one row of Table A (compounds I.A.128aA-1 to I.A.128aA-366)

### Table 129a

Compounds I.A in which Z is (E) CH₂C(CH₃)═CHCH₂, R², R³ and R⁴ are H, R⁵ is C(CH₃)(CH₂CH₃)₂ and the combination of R¹ and Y corresponds in each case to one row of Table A (compounds I.A.129aA-1 to I.A.129aA-366)

### Table 130a

Compounds I.A in which Z is (E) CH₂CH═C(CH₃)CH₂, R², R³ and R⁴ are H, R⁵ is C(CH₃)(CH₂CH₃)₂ and the combination of R¹ and Y corresponds in each case to one row of Table A (compounds I.A.130aA-1 to I.A.130aA-366)

### Table 131a

Compounds I.A in which Z is (E) CH₂C(CH₃)═C(CH₃)CH₂, R², R³ and R⁴ are H, R⁵ is C(CH₃)(CH₂CH₃)₂ and the combination of R¹ and Y corresponds in each case to one row of Table A (compounds I.A.131aA-1 to I.A.131aA-366)

### Table 132a

Compounds I.A in which Z is (E) C(CH₃)═C(CH₃)(CH₂)₂CH₂, R², R³ and R⁴ are H, R⁵ is C(CH₃)(CH₂CH₃)₂ and the combination of R¹ and Y corresponds in each case to one row of Table A (compounds I.A.132aA-1 to I.A.132aA-366)

### Table 133a

Compounds I.A in which Z is (E) C(CH₃)═CH(CH₂)₂CH₂, R², R³ and R⁴ are H, R⁵ is C(CH₃)(CH₂CH₃)₂ and the combination of R¹ and Y corresponds in each case to one row of Table A (compounds I.A.133aA-1 to I.A.133aA-366)

### Table 134a

Compounds I.A in which Z is (E) CH═C(CH₃)(CH₂)₂CH₂, R², R³ and R⁴ are H, R⁵ is C(CH₃)(CH₂CH₃)₂ and the combination of R¹ and Y corresponds in each case to one row of Table A (compounds I.A.134aA-1 to I.A.134aA-366)

### Table 135a

Compounds I.A in which Z is CH₂C═CCH₂, R², R³ and R⁴ are H, R⁵ is C(CH₃)(CH₂CH₃)₂ and the combination of R¹ and Y corresponds in each case to one row of Table A (compounds I.A.135aA-1 to I.A.135aA-366)

### Table 136a

Compounds I.A in which Z is CH₂CH₂CH₂, R², R³ and R⁴ are H, R⁵ is C(CH₃)₂CH(CH₃)₂ and the combination of R¹ and Y corresponds in each case to one row of Table A (compounds I.A.136aA-1 to I.A.136aA-366)

### Table 137a

Compounds I.A in which Z is CH₂(CH₂)₂CH₂, R², R³ and R⁴ are H, R⁵ is C(CH₃)₂CH(CH₃)₂ and the combination of R¹ and Y corresponds in each case to one row of Table A (compounds I.A.137aA-1 to I.A.137aA-366)

### Table 138a

Compounds I.A in which Z is CH₂(CH₂)₃CH₂, R², R³ and R⁴ are H, R⁵ is C(CH₃)₂CH(CH₃)₂ and the combination of R¹ and Y corresponds in each case to one row of Table A (compounds I.A.138aA-1 to I.A.138aA-366)

### Table 139a

Compounds I.A in which Z is CH₂CH(CH₃)CH₂, R², R³ and R⁴ are H, R⁵ is C(CH₃)₂CH(CH₃)₂ and the combination of R¹ and Y corresponds in each case to one row of Table A (compounds I.A.139aA-1 to I.A.139aA-366)

### Table 140a

Compounds I.A in which Z is CH₂CH₂CH(CH₃), R², R³ and R⁴ are H, R⁵ is C(CH₃)₂CH(CH₃)₂ and the combination of R¹ and Y corresponds in each case to one row of Table A (compounds I.A.140aA-1 to I.A.140aA-366)

### Table 141a

Compounds I.A in which Z is CH(CH₃)CH₂CH₂, R², R³ and R⁴ are H, R⁵ is C(CH₃)₂CH(CH₃)₂ and the combination of R¹ and Y corresponds in each case to one row of Table A (compounds I.A.141aA-1 to I.A.141aA-366)

### Table 142a

Compounds I.A in which Z is CH₂C(CH₃)₂CH₂, R², R³ and R⁴ are H, R⁵ is C(CH₃)₂CH(CH₃)₂ and the combination of R¹ and Y corresponds in each case to one row of Table A (compounds I.A.142aA-1 to I.A.142aA-366)

### Table 143a

Compounds I.A in which Z is CH₂C(CH₂CH₂)CH₂, R², R³ and R⁴ are H, R⁵ is C(CH₃)₂CH(CH₃)₂ and the combination of R¹ and Y corresponds in each case to one row of Table A (compounds I.A.143aA-1 to I.A.143aA-366)

### Table 144a

Compounds I.A in which Z is C(CH₂CH₂)CH₂CH₂, R², R³ and R⁴ are H, R⁵ is C(CH₃)₂CH(CH₃)₂ and the combination of R¹ and Y corresponds in each case to one row of Table A (compounds I.A.144aA-1 to I.A.144aA-366)

### Table 145a

Compounds I.A in which Z is CH₂CH(CH₃)(CH₂)₂CH₂, R², R³ and R⁴ are H, R⁵ is C(CH₃)₂CH(CH₃)₂ and the combination of R¹ and Y corresponds in each case to one row of Table A (compounds I.A.145aA-1 to I.A.145aA-366)

### Table 146a

Compounds I.A in which Z is C(CH₃)₂(CH₂)₃CH₂, R², R³ and R⁴ are H, R⁵ is C(CH₃)₂CH(CH₃)₂ and the combination of R¹ and Y corresponds in each case to one row of Table A (compounds I.A.146aA-1 to I.A.146aA-366)

### Table 147a

Compounds I.A in which Z is C(CH₂CH₂)(CH₂)₃CH₂, R², R³ and R⁴ are H, R⁵ is C(CH₃)₂CH(CH₃)₂ and the combination of R¹ and Y corresponds in each case to one row of Table A (compounds I.A.147aA-1 to I.A.147aA-366)

### Table 148a

Compounds I.A in which Z is CH₂CH₂CH(CH₃)CH₂CH₂, R², R³ and R⁴ are H, R⁵ is C(CH₃)₂CH(CH₃)₂ and the combination of R¹ and Y corresponds in each case to one row of Table A (compounds I.A.148aA-1 to I.A.148aA-366)

### Table 149a

Compounds I.A in which Z is CH₂CH₂CH₂CH(CH₃)CH₂, R², R³ and R⁴ are H, R⁵ is C(CH₃)₂CH(CH₃)₂ and the combination of R¹ and Y corresponds in each case to one row of Table A (compounds I.A.149aA-1 to I.A.149aA-366)

### Table 150a

Compounds I.A in which Z is CH₂(CH₂)₃CH(CH₃), R², R³ and R⁴ are H, R⁵ is C(CH₃)₂CH(CH₃)₂ and the combination of R¹ and Y corresponds in each case to one row of Table A (compounds I.A.150aA-1 to I.A.150aA-366)

### Table 151a

Compounds I.A in which Z is (E) CH═CHCH₂, R², R³ and R⁴ are H, R⁵ is C(CH₃)₂CH(CH₃)₂ and the combination of R¹ and Y corresponds in each case to one row of Table A (compounds I.A.151aA-1 to I.A.151aA-366)

### Table 152a

Compounds I.A in which Z is (E) C(CH₃)═CHCH₂, R², R³ and R⁴ are H, R⁵ is C(CH₃)₂CH(CH₃)₂ and the combination of R¹ and Y corresponds in each case to one row of Table A (compounds I.A.152aA-1 to I.A.152aA-366)

### Table 153a

Compounds I.A in which Z is (E) C(CH₃)═C(CH₃)CH₂, R², R³ and R⁴ are H, R⁵ is C(CH₃)₂CH(CH₃)₂ and the combination of R¹ and Y corresponds in each case to one row of Table A (compounds I.A.153aA-1 to I.A.153aA-366)

### Table 154a

Compounds I.A in which Z is (E) CH═C(CH₃)CH₂, R², R³ and R⁴ are H, R⁵ is C(CH₃)₂CH(CH₃)₂ and the combination of R¹ and Y corresponds in each case to one row of Table A (compounds I.A.154aA-1 to I.A.154aA-366)

### Table 155a

Compounds I.A in which Z is (E) CH₂CH═CHCH₂, R², R³ and R⁴ are H, R⁵ is C(CH₃)₂CH(CH₃)₂ and the combination of R¹ and Y corresponds in each case to one row of Table A (compounds I.A.155aA-1 to I.A.155aA-366)

### Table 156a

Compounds I.A in which Z is (E) CH₂C(CH₃)═CHCH₂, R², R³ and R⁴ are H, R⁵ is C(CH₃)₂CH(CH₃)₂ and the combination of R¹ and Y corresponds in each case to one row of Table A (compounds I.A.156aA-1 to I.A.156aA-366)

### Table 157a

Compounds I.A in which Z is (E) CH₂CH═C(CH₃)CH₂, R², R³ and R⁴ are H, R⁵ is C(CH₃)₂CH(CH₃)₂ and the combination of R¹ and Y corresponds in each case to one row of Table A (compounds I.A.157aA-1 to I.A.157aA-366)

### Table 158a

Compounds I.A in which Z is (E) CH₂C(CH₃)═C(CH₃)CH₂, R², R³ and R⁴ are H, R⁵ is C(CH₃)₂CH(CH₃)₂ and the combination of R¹ and Y corresponds in each case to one row of Table A (compounds I.A.158aA-1 to I.A.158aA-366)

### Table 159a

Compounds I.A in which Z is (E) C(CH₃)═C(CH₃)(CH₂)₂CH₂, R², R³ and R⁴ are H, R⁵ is C(CH₃)₂CH(CH₃)₂ and the combination of R¹ and Y corresponds in each case to one row of Table A (compounds I.A.159aA-1 to I.A.159aA-366)

### Table 160a

Compounds I.A in which Z is (E) C(CH₃)═CH(CH₂)₂CH₂, R², R³ and R⁴ are H, R⁵ is C(CH₃)₂CH(CH₃)₂ and the combination of R¹ and Y corresponds in each case to one row of Table A (compounds I.A.160aA-1 to I.A.160aA-366)

### Table 161a

Compounds I.A in which Z is (E) CH═C(CH₃)(CH₂)₂CH₂, R², R³ and R⁴ are H, R⁵ is C(CH₃)₂CH(CH₃)₂ and the combination of R¹ and Y corresponds in each case to one row of Table A (compounds I.A.161aA-1 to I.A.161aA-366)

### Table 162a

Compounds I.A in which Z is CH₂C═CCH₂, R², R³ and R⁴ are H, R⁵ is C(CH₃)₂CH(CH₃)₂ and the combination of R¹ and Y corresponds in each case to one row of Table A (compounds I.A.162aA-1 to I.A.162aA-366)

### Table 163a

Compounds I.A in which Z is CH₂CH₂CH₂, R², R³ and R⁴ are H, R⁵ is C(CH₃)₂CH(CH₃)₃ and the combination of R¹ and Y corresponds in each case to one row of Table A (compounds I.A.163aA-1 to I.A.163aA-366)

### Table 164a

Compounds I.A in which Z is CH₂(CH₂)₂CH₂, R², R³ and R⁴ are H, R⁵ is C(CH₃)₂CH(CH₃)₃ and the combination of R¹ and Y corresponds in each case to one row of Table A (compounds I.A.164aA-1 to I.A.164aA-366)

### Table 165a

Compounds I.A in which Z is CH₂(CH₂)₂CH₂, R², R³ and R⁴ are H, R⁵ is C(CH₃)₂CH(CH₃)₃ and the combination of R¹ and Y corresponds in each case to one row of Table A (compounds I.A.165aA-1 to I.A.165aA-366)

### Table 166a

Compounds I.A in which Z is CH₂CH(CH₃)CH₂, R², R³ and R⁴ are H, R⁵ is C(CH₃)₂CH(CH₃)₃ and the combination of R¹ and Y corresponds in each case to one row of Table A (compounds I.A.166aA-1 to I.A.166aA-366)

### Table 167a

Compounds I.A in which Z is CH₂CH₂CH(CH₃), R², R³ and R⁴ are H, R⁵ is C(CH₃)₂C(CH₃)₃ and the combination of R¹ and Y corresponds in each case to one row of Table A (compounds I.A.167aA-1 to I.A.167aA-366)

### Table 168a

Compounds I.A in which Z is CH(CH₃)CH₂CH₂, R², R³ and R⁴ are H, R⁵ is C(CH₃)₂CH(CH₃)₃ and the combination of R¹ and Y corresponds in each case to one row of Table A (compounds I.A.168aA-1 to I.A.168aA-366)

### Table 169a

Compounds I.A in which Z is CH₂C(CH₃)₂CH₂, R², R³ and R⁴ are H, R⁵ is C(CH₃)₂CH(CH₃)₃ and the combination of R¹ and Y corresponds in each case to one row of Table A (compounds I.A.169aA-1 to I.A.169aA-366)

### Table 170a

Compounds I.A in which Z is CH₂C(CH₂CH₂)CH₂, R², R³ and R⁴ are H, R⁵ is C(CH₃)₂CH(CH₃)₃ and the combination of R¹ and Y corresponds in each case to one row of Table A (compounds I.A.170aA-1 to I.A.170aA-366)

### Table 171a

Compounds I.A in which Z is C(CH₂CH₂)CH₂CH₂, R², R³ and R⁴ are H, R⁵ is C(CH₃)₂C(CH₃)₃ and the combination of R¹ and Y corresponds in each case to one row of Table A (compounds I.A.171aA-1 to I.A.171aA-366)

### Table 172a

Compounds I.A in which Z is CH₂CH(CH₃)(CH₂)₂CH₂, R², R³ and R⁴ are H, R⁵ is C(CH₃)₂C(CH₃)₃ and the combination of R¹ and Y corresponds in each case to one row of Table A (compounds I.A.172aA-1 to I.A.172aA-366)

### Table 173a

Compounds I.A in which Z is C(CH₃)₂(CH₂)₃CH₂, R², R³ and R⁴ are H, R⁵ is C(CH₃)₂C(CH₃)₃ and the combination of R¹ and Y corresponds in each case to one row of Table A (compounds I.A.173aA-1 to I.A.173aA-366)

### Table 174a

Compounds I.A in which Z is C(CH₂CH₂)(CH₂)₃CH₂, R², R³ and R⁴ are H, R⁵ is C(CH₃)₂C(CH₃)₃ and the combination of R¹ and Y corresponds in each case to one row of Table A (compounds I.A.174aA-1 to I.A.174aA-366)

### Table 175a

Compounds I.A in which Z is CH₂CH₂CH(CH₃)CH₂CH₂, R², R³ and R⁴ are H, R⁵ is C(CH₃)₂C(CH₃)₃ and the combination of R¹ and Y corresponds in each case to one row of Table A (compounds I.A.175aA-1 to I.A.175aA-366)

### Table 176a

Compounds I.A in which Z is CH₂CH₂CH₂CH(CH₃)CH₂, R², R³ and R⁴ are H, R⁵ is C(CH₃)₂C(CH₃)₃ and the combination of R¹ and Y corresponds in each case to one row of Table A (compounds I.A.176aA-1 to I.A.176aA-366)

### Table 177a

Compounds I.A in which Z is CH₂(CH₂)₃CH(CH₃), R², R³ and R⁴ are H, R⁵ is C(CH₃)₂C(CH₃)₃ and the combination of R¹ and Y corresponds in each case to one row of Table A (compounds I.A.177aA-1 to I.A.177aA-366)

### Table 178a

Compounds I.A in which Z is (E) CH═CHCH₂, R², R³ and R⁴ are H, R⁵ is C(CH₃)₂C(CH₃)₃ and the combination of R¹ and Y corresponds in each case to one row of Table A (compounds I.A.178aA-1 to I.A.178aA-366)

### Table 179a

Compounds I.A in which Z is (E) C(CH₃)═CHCH₂, R², R³ and R⁴ are H, R⁵ is C(CH₃)₂C(CH₃)₃ and the combination of R¹ and Y corresponds in each case to one row of Table A (compounds I.A.179aA-1 to I.A.179aA-366)

### Table 180a

Compounds I.A in which Z is (E) C(CH₃)═C(CH₃)CH₂, R², R³ and R⁴ are H, R⁵ is C(CH₃)₂C(CH₃)₃ and the combination of R¹ and Y corresponds in each case to one row of Table A (compounds I.A.180aA-1 to I.A.180aA-366)

### Table 181a

Compounds I.A in which Z is (E) CH═C(CH₃)CH₂, R², R³ and R⁴ are H, R⁵ is C(CH₃)₂C(CH₃)₃ and the combination of R¹ and Y corresponds in each case to one row of Table A (compounds I.A.181aA-1 to I.A.181aA-366)

### Table 182a

Compounds I.A in which Z is (E) CH₂CH═CHCH₂, R², R³ and R⁴ are H, R⁵ is C(CH₃)₂C(CH₃)₃ and the combination of R¹ and Y corresponds in each case to one row of Table A (compounds I.A.182aA-1 to I.A.182aA-366)

### Table 183a

Compounds I.A in which Z is (E) CH₂C(CH₃)═CHCH₂, R², R³ and R⁴ are H, R⁵ is C(CH₃)₂C(CH₃)₃ and the combination of R¹ and Y corresponds in each case to one row of Table A (compounds I.A.183aA-1 to I.A.183aA-366)

### Table 184a

Compounds I.A in which Z is (E) CH₂CH═C(CH₃)CH₂, R², R³ and R⁴ are H, R⁵ is C(CH₃)₂C(CH₃)₃ and the combination of R¹ and Y corresponds in each case to one row of Table A (compounds I.A.184aA-1 to I.A.184aA-366)

### Table 185a

Compounds I.A in which Z is (E) CH₂C(CH₃)═C(CH₃)CH₂, R², R³ and R⁴ are H, R⁵ is C(CH₃)₂C(CH₃)₃ and the combination of R¹ and Y corresponds in each case to one row of Table A (compounds I.A.185aA-1 to I.A.185aA-366)

### Table 186a

Compounds I.A in which Z is (E) C(CH₃)═C(CH₃)(CH₂)₂CH₂, R², R³ and R⁴ are H, R⁵ is C(CH₃)₂C(CH₃)₃ and the combination of R¹ and Y corresponds in each case to one row of Table A (compounds I.A.186aA-1 to I.A.186aA-366)

### Table 187a

Compounds I.A in which Z is (E) C(CH₃)═CH(CH₂)₂CH₂, R², R³ and R⁴ are H, R⁵ is C(CH₃)₂C(CH₃)₃ and the combination of R¹ and Y corresponds in each case to one row of Table A (compounds I.A.187aA-1 to I.A.187aA-366)

### Table 188a

Compounds I.A in which Z is (E) CH═C(CH₃)(CH₂)₂CH₂, R², R³ and R⁴ are H, R⁵ is C(CH₃)₂C(CH₃)₃ and the combination of R¹ and Y corresponds in each case to one row of Table A (compounds I.A.188aA-1 to I.A.188aA-366)

### Table 189a

Compounds I.A in which Z is CH₂C≡CCH₂, R², R³ and R⁴ are H, R⁵ is C(CH₃)₂C(CH₃)₃ and the combination of R¹ and Y corresponds in each case to one row of Table A (compounds I.A.189aA-1 to I.A.189aA-366)

### Table 190a

Compounds I.A in which Z is CH₂CH₂CH₂, R², R³ and R⁴ are H, R⁵ is CH(CH₃)C(CH₃)₃ and the combination of R¹ and Y corresponds in each case to one row of Table A (compounds I.A.190aA-1 to I.A.190aA-366)

### Table 191a

Compounds I.A in which Z is CH₂(CH₂)₂CH₂, R², R³ and R⁴ are H, R⁵ is CH(CH₃)C(CH₃)₃ and the combination of R¹ and Y corresponds in each case to one row of Table A (compounds I.A.191aA-1 to I.A.191aA-366)

### Table 192a

Compounds I.A in which Z is CH₂(CH₂)₃CH₂, R², R³ and R⁴ are H, R⁵ is CH(CH₃)C(CH₃)₃ and the combination of R¹ and Y corresponds in each case to one row of Table A (compounds I.A.192aA-1 to I.A.192aA-366)

### Table 193a

Compounds I.A in which Z is CH₂CH(CH₃)CH₂, R², R³ and R⁴ are H, R⁵ is CH(CH₃)C(CH₃)₃ and the combination of R¹ and Y corresponds in each case to one row of Table A (compounds I.A.193aA-1 to I.A.193aA-366)

### Table 194a

Compounds I.A in which Z is CH₂CH₂CH(CH₃), R², R³ and R⁴ are H, R⁵ is CH(CH₃)C(CH₃)₃ and the combination of R¹ and Y corresponds in each case to one row of Table A (compounds I.A.194aA-1 to I.A.194aA-366)

### Table 195a

Compounds I.A in which Z is CH(CH₃)CH₂CH₂, R², R³ and R⁴ are H, R⁵ is CH(CH₃)C(CH₃)₃ and the combination of R¹ and Y corresponds in each case to one row of Table A (compounds I.A.195aA-1 to I.A.195aA-366)

### Table 196a

Compounds I.A in which Z is CH₂C(CH₃)₂CH₂, R², R³ and R⁴ are H, R⁵ is CH(CH₃)C(CH₃)₃ and the combination of R¹ and Y corresponds in each case to one row of Table A (compounds I.A.196aA-1 to I.A.196aA-366)

### Table 197a

Compounds I.A in which Z is CH₂C(CH₂CH₂)CH₂, R², R³ and R⁴ are H, R⁵ is CH(CH₃)C(CH₃)₃ and the combination of R¹ and Y corresponds in each case to one row of Table A (compounds I.A.197aA-1 to I.A.197aA-366)

### Table 198a

Compounds I.A in which Z is C(CH₂CH₂)CH₂CH₂, R², R³ and R⁴ are H, R⁵ is CH(CH₃)C(CH₃)₃ and the combination of R¹ and Y corresponds in each case to one row of Table A (compounds I.A.198aA-1 to I.A.198aA-366)

### Table 199a

Compounds I.A in which Z is CH₂CH(CH₃)(CH₂)₂CH₂, R², R³ and R⁴ are H, R⁵ is CH(CH₃)C(CH₃)₃ and the combination of R¹ and Y corresponds in each case to one row of Table A (compounds I.A.199aA-1 to I.A.199aA-366)

### Table 200a

Compounds I.A in which Z is C(CH₃)₂(CH₂)₃CH₂, R², R³ and R⁴ are H, R⁵ is CH(CH₃)C(CH₃)₃ and the combination of R¹ and Y corresponds in each case to one row of Table A (compounds I.A.200aA-1 to I.A.200aA-366)

### Table 201a

Compounds I.A in which Z is C(CH₂CH₂)(CH₂)₃CH₂, R², R³ and R⁴ are H, R⁵ is CH(CH₃)C(CH₃)₃ and the combination of R¹ and Y corresponds in each case to one row of Table A (compounds I.A.201aA-1 to I.A.201aA-366)

### Table 202a

Compounds I.A in which Z is CH₂CH₂CH(CH₃)CH₂CH₂, R², R³ and R⁴ are H, R⁵ is CH(CH₃)C(CH₃)₃ and the combination of R¹ and Y corresponds in each case to one row of Table A (compounds I.A.202aA-1 to I.A.202aA-366)

### Table 203a

Compounds I.A in which Z is CH₂CH₂CH₂CH(CH₃)CH₂, R², R³ and R⁴ are H, R⁵ is CH(CH₃)C(CH₃)₃ and the combination of R¹ and Y corresponds in each case to one row of Table A (compounds I.A.203aA-1 to I.A.203aA-366)

### Table 204a

Compounds I.A in which Z is CH₂(CH₂)₃CH(CH₃), R², R³ and R⁴ are H, R⁵ is CH(CH₃)C(CH₃)₃ and the combination of R¹ and Y corresponds in each case to one row of Table A (compounds I.A.204aA-1 to I.A.204aA-366)

### Table 205a

Compounds I.A in which Z is (E) CH═CHCH₂, R², R³ and R⁴ are H, R⁵ is CH(CH₃)C(CH₃)₃ and the combination of R¹ and Y corresponds in each case to one row of Table A (compounds I.A.205aA-1 to I.A.205aA-366)

### Table 206a

Compounds I.A in which Z is (E) C(CH₃)═CHCH₂, R², R³ and R⁴ are H, R⁵ is CH(CH₃)C(CH₃)₃ and the combination of R¹ and Y corresponds in each case to one row of Table A (compounds I.A.206aA-1 to I.A.206aA-366)

### Table 207a

Compounds I.A in which Z is (E) C(CH₃)═C(CH₃)CH₂, R², R³ and R⁴ are H, R⁵ is CH(CH₃)C(CH₃)₃ and the combination of R¹ and Y corresponds in each case to one row of Table A (compounds I.A.207aA-1 to I.A.207aA-366)

### Table 208a

Compounds I.A in which Z is (E) CH═C(CH₃)CH₂, R², R³ and R⁴ are H, R⁵ is CH(CH₃)C(CH₃)₃ and the combination of R¹ and Y corresponds in each case to one row of Table A (compounds I.A.208aA-1 to I.A.208aA-366)

### Table 209a

Compounds I.A in which Z is (E) CH₂CH═CHCH₂, R², R³ and R⁴ are H, R⁵ is CH(CH₃)C(CH₃)₃ and the combination of R¹ and Y corresponds in each case to one row of Table A (compounds I.A.209aA-1 to I.A.209aA-366)

### Table 210a

Compounds I.A in which Z is (E) CH₂C(CH₃)═CHCH₂, R², R³ and R⁴ are H, R⁵ is CH(CH₃)C(CH₃)₃ and the combination of R¹ and Y corresponds in each case to one row of Table A (compounds I.A.210aA-1 to I.A.210aA-366)

### Table 211a

Compounds I.A in which Z is (E) CH₂CH═C(CH₃)CH₂, R², R³ and R⁴ are H, R⁵ is CH(CH₃)C(CH₃)₃ and the combination of R¹ and Y corresponds in each case to one row of Table A (compounds I.A.211aA-1 to I.A.211aA-366)

### Table 212a

Compounds I.A in which Z is (E) CH₂C(CH₃)═C(CH₃)CH₂, R², R³ and R⁴ are H, R⁵ is CH(CH₃)C(CH₃)₃ and the combination of R¹ and Y corresponds in each case to one row of Table A (compounds I.A.212aA-1 to I.A.212aA-366)

### Table 213a

Compounds I.A in which Z is (E) C(CH₃)═C(CH₃)(CH₂)₂CH₂, R², R³ and R⁴ are H, R⁵ is CH(CH₃)C(CH₃)₃ and the combination of R¹ and Y corresponds in each case to one row of Table A (compounds I.A.213aA-1 to I.A.213aA-366)

### Table 214a

Compounds I.A in which Z is (E) C(CH₃)═CH(CH₂)₂CH₂, R², R³ and R⁴ are H, R⁵ is CH(CH₃)C(CH₃)₃ and the combination of R¹ and Y corresponds in each case to one row of Table A (compounds I.A.214aA-1 to I.A.214aA-366)

### Table 215a

Compounds I.A in which Z is (E) CH═C(CH₃)(CH₂)₂CH₂, R², R³ and R⁴ are H, R⁵ is CH(CH₃)C(CH₃)₃ and the combination of R¹ and Y corresponds in each case to one row of Table A (compounds I.A.215aA-1 to IA.215aA-366)

### Table 216a

Compounds I.A in which Z is CH₂C═CH₂, R², R³ and R⁴ are H, R⁵ is CH(CH₃)C(CH₃)₃ and the combination of R¹ and Y corresponds in each case to one row of Table A (compounds I.A.216aA-1 to I.A.216aA-366)

### Table 217a

Compounds I.A in which Z is CH₂CH₂CH₂, R², R³ and R⁴ are H, R⁵ is CH(CH₃)CH(CH₃)₂ and the combination of R¹ and Y corresponds in each case to one row of Table A (compounds I.A.217aA-1 to I.A.217aA-366)

### Table 218a

Compounds I.A in which Z is CH₂(CH₂)₂CH₂, R², R³ and R⁴ are H, R⁵ is CH(CH₃)CH(CH₃)₂ and the combination of R¹ and Y corresponds in each case to one row of Table A (compounds I.A.218aA-1 to I.A.218aA-366)

### Table 219a

Compounds I.A in which Z is CH₂(CH₂)₃CH₂, R², R³ and R⁴ are H, R⁵ is CH(CH₃)CH(CH₃)₂ and the combination of R¹ and Y corresponds in each case to one row of Table A (compounds I.A.219aA-1 to I.A.219aA-366)

### Table 220a

Compounds I.A in which Z is CH₂CH(CH₃)CH₂, R², R³ and R⁴ are H, R⁵ is CH(CH₃)CH(CH₃)₂ and the combination of R¹ and Y corresponds in each case to one row of Table A (compounds I.A.220aA-1 to I.A.220aA-366)

### Table 221a

Compounds I.A in which Z is CH₂CH₂CH(CH₃), R², R³ and R⁴ are H, R⁵ is CH(CH₃)CH(CH₃)₂ and the combination of R¹ and Y corresponds in each case to one row of Table A (compounds I.A.221aA-1 to I.A.221aA-366)

### Table 222a

Compounds I.A in which Z is CH(CH₃)CH₂CH₂, R², R³ and R⁴ are H, R⁵ is CH(CH₃)CH(CH₃)₂ and the combination of R¹ and Y corresponds in each case to one row of Table A (compounds I.A.222aA-1 to I.A.222aA-366)

### Table 223a

Compounds I.A in which Z is CH₂C(CH₃)₂CH₂, R², R³ and R⁴ are H, R⁵ is CH(CH₃)CH(CH₃)₂ and the combination of R¹ and Y corresponds in each case to one row of Table A (compounds I.A.223aA-1 to I.A.223aA-366)

### Table 224a

Compounds I.A in which Z is CH₂C(CH₂CH₂)CH₂, R², R³ and R⁴ are H, R⁵ is CH(CH₃)CH(CH₃)₂ and the combination of R¹ and Y corresponds in each case to one row of Table A (compounds I.A.224aA-1 to I.A.224aA-366)

### Table 225a

Compounds I.A in which Z is C(CH₂CH₂)CH₂CH₂, R², R³ and R⁴ are H, R⁵ is CH(CH₃)CH(CH₃)₂ and the combination of R¹ and Y corresponds in each case to one row of Table A (compounds I.A.225aA-1 to I.A.225aA-366)

### Table 226a

Compounds I.A in which Z is CH₂CH(CH₃)(CH₂)₂CH₂, R², R³ and R⁴ are H, R⁵ is CH(CH₃)CH(CH₃)₂ and the combination of R¹ and Y corresponds in each case to one row of Table A (compounds I.A.226aA-1 to I.A.226aA-366)

### Table 227a

Compounds I.A in which Z is C(CH₃)₂(CH₂)₃CH₂, R², R³ and R⁴ are H, R⁵ is CH(CH₃)CH(CH₃)₂ and the combination of R¹ and Y corresponds in each case to one row of Table A (compounds I.A.227aA-1 to I.A.227aA-366)

### Table 228a

Compounds I.A in which Z is C(CH₂CH₂)(CH₂)₃CH₂, R², R³ and R⁴ are H, R⁵ is CH(CH₃)CH(CH₃)₂ and the combination of R¹ and Y corresponds in each case to one row of Table A (compounds I.A.228aA-1 to I.A.228aA-366)

### Table 229a

Compounds I.A in which Z is CH₂CH₂CH(CH₃)CH₂CH₂, R², R³ and R⁴ are H, R⁵ is CH(CH₃)CH(CH₃)₂ and the combination of R¹ and Y corresponds in each case to one row of Table A (compounds I.A.229aA-1 to I.A.229aA-366)

### Table 230a

Compounds I.A in which Z is CH₂CH₂CH₂CH(CH₃)CH₂, R², R³ and R⁴ are H, R⁵ is CH(CH₃)CH(CH₃)₂ and the combination of R¹ and Y corresponds in each case to one row of Table A (compounds I.A.230aA-1 to I.A.230aA-366)

### Table 231a

Compounds I.A in which Z is CH₂(CH₂)₃CH(CH₃), R², R³ and R⁴ are H, R⁵ is CH(CH₃)CH(CH₃)₂ and the combination of R¹ and Y corresponds in each case to one row of Table A (compounds I.A.231aA-1 to I.A.231aA-366)

### Table 232a

Compounds I.A in which Z is (E) CH═CHCH₂, R², R³ and R⁴ are H, R⁵ is CH(CH₃)CH(CH₃)₂ and the combination of R¹ and Y corresponds in each case to one row of Table A (compounds I.A.232aA-1 to I.A.232aA-366)

### Table 233a

Compounds I.A in which Z is (E) C(CH₃)═CHCH₂, R², R³ and R⁴ are H, R⁵ is CH(CH₃)CH(CH₃)₂ and the combination of R¹ and Y corresponds in each case to one row of Table A (compounds I.A.233aA-1 to I.A.233aA-366)

### Table 234a

Compounds I.A in which Z is (E) C(CH₃)═C(CH₃)CH₂, R², R³ and R⁴ are H, R⁵ is CH(CH₃)CH(CH₃)₂ and the combination of R¹ and Y corresponds in each case to one row of Table A (compounds I.A.234aA-1 to I.A.234aA-366)

### Table 235a

Compounds I.A in which Z is (E) CH═C(CH₃)CH₂, R², R³ and R⁴ are H, R⁵ is CH(CH₃)CH(CH₃)₂ and the combination of R¹ and Y corresponds in each case to one row of Table A (compounds I.A.235aA-1 to I.A.235aA-366)

### Table 236a

Compounds I.A in which Z is (E) CH₂CH═CHCH₂, R², R³ and R⁴ are H, R⁵ is CH(CH₃)CH(CH₃)₂ and the combination of R¹ and Y corresponds in each case to one row of Table A (compounds I.A.236aA-1 to I.A.236aA-366)

### Table 237a

Compounds I.A in which Z is (E) CH₂C(CH₃)═CHCH₂, R², R³ and R⁴ are H, R⁵ is CH(CH₃)CH(CH₃)₂ and the combination of R¹ and Y corresponds in each case to one row of Table A (compounds I.A.237aA-1 to I.A.237aA-366)

### Table 238a

Compounds I.A in which Z is (E) CH₂CH═C(CH₃)CH₂, R², R³ and R⁴ are H, R⁵ is CH(CH₃)CH(CH₃)₂ and the combination of R¹ and Y corresponds in each case to one row of Table A (compounds I.A.238aA-1 to I.A.238aA-366)

### Table 239a

Compounds I.A in which Z is (E) CH₂C(CH₃)═C(CH₃)CH₂, R², R³ and R⁴ are H, R⁵ is CH(CH₃)CH(CH₃)₂ and the combination of R¹ and Y corresponds in each case to one row of Table A (compounds I.A.239aA-1 to I.A.239aA-366)

### Table 240a

Compounds I.A in which Z is (E) C(CH₃)═C(CH₃)(CH₂)₂CH₂, R², R³ and R⁴ are H, R⁵ is CH(CH₃)CH(CH₃)₂ and the combination of R¹ and Y corresponds in each case to one row of Table A (compounds I.A.240aA-1 to I.A.240aA-366)

### Table 241a

Compounds I.A in which Z is (E) C(CH₃)═CH(CH₂)₂CH₂, R², R³ and R⁴ are H, R⁵ is CH(CH₃)CH(CH₃)₂ and the combination of R¹ and Y corresponds in each case to one row of Table A (compounds I.A.241aA-1 to I.A.241aA-366)

### Table 242a

Compounds I.A in which Z is (E) CH═C(CH₃)(CH₂)₂CH₂, R², R³ and R⁴ are H, R⁵ is CH(CH₃)CH(CH₃)₂ and the combination of R¹ and Y corresponds in each case to one row of Table A (compounds I.A.242aA-1 to I.A.242aA-366)

### Table 243a

Compounds I.A in which Z is CH₂C≡CCH₂, R², R³ and R⁴ are H, R⁵ is CH(CH₃)CH(CH₃)₂ and the combination of R¹ and Y corresponds in each case to one row of Table A (compounds I.A.243aA-1 to I.A.243aA-366)

### Table 244a

Compounds I.A in which Z is CH₂CH₂CH₂, R², R³ and R⁴ are H, R⁵ is CH₂CH(CH₃)₂ and the combination of R¹ and Y corresponds in each case to one row of Table A (compounds I.A.244aA-1 to I.A.244aA-366)

### Table 245a

Compounds I.A in which Z is CH₂(CH₂)₂CH₂, R², R³ and R⁴ are H, R⁵ is CH₂CH(CH₃)₂ and the combination of R¹ and Y corresponds in each case to one row of Table A (compounds I.A.245aA-1 to I.A.245aA-366)

### Table 246a

Compounds I.A in which Z is CH₂(CH₂)₃CH₂, R², R³ and R⁴ are H, R⁵ is CH₂CH(CH₃)₂ and the combination of R¹ and Y corresponds in each case to one row of Table A (compounds I.A.246aA-1 to I.A.246aA-366)

### Table 247a

Compounds I.A in which Z is CH₂CH(CH₃)CH₂, R², R³ and R⁴ are H, R⁵ is CH₂CH(CH₃)₂ and the combination of R¹ and Y corresponds in each case to one row of Table A (compounds I.A.247aA-1 to I.A.247aA-366)

### Table 248a

Compounds I.A in which Z is CH₂CH₂CH(CH₃), R², R³ and R⁴ are H, R⁵ is CH₂CH(CH₃)₂ and the combination of R¹ and Y corresponds in each case to one row of Table A (compounds I.A.248aA-1 to I.A.248aA-366)

### Table 249a

Compounds I.A in which Z is CH(CH₃)CH₂CH₂, R², R³ and R⁴ are H, R⁵ is CH₂CH(CH₃)₂ and the combination of R¹ and Y corresponds in each case to one row of Table A (compounds I.A.249aA-1 to I.A.249aA-366)

### Table 250a

Compounds I.A in which Z is CH₂C(CH₃)₂CH₂, R², R³ and R⁴ are H, R⁵ is CH₂CH(CH₃)₂ and the combination of R¹ and Y corresponds in each case to one row of Table A (compounds I.A.250aA-1 to I.A.250aA-366)

### Table 251a

Compounds I.A in which Z is CH₂C(CH₂CH₂)CH₂, R², R³ and R⁴ are H, R⁵ is CH₂CH(CH₃)₂ and the combination of R¹ and Y corresponds in each case to one row of Table A (compounds I.A.251aA-1 to I.A.251aA-366)

### Table 252a

Compounds I.A in which Z is C(CH₂CH₂)CH₂CH₂, R², R³ and R⁴ are H, R⁵ is CH₂CH(CH₃)₂ and the combination of R¹ and Y corresponds in each case to one row of Table A (compounds I.A.252aA-1 to I.A.252aA-366)

### Table 253a

Compounds I.A in which Z is CH₂CH(CH₃)(CH₂)₂CH₂, R², R³ and R⁴ are H, R⁵ is CH₂CH(CH₃)₂ and the combination of R¹ and Y corresponds in each case to one row of Table A (compounds I.A.253aA-1 to I.A.253aA-366)

### Table 254a

Compounds I.A in which Z is C(CH₃)₂(CH₂)₃CH₂, R², R³ and R⁴ are H, R⁵ is CH₂CH(CH₃)₂ and the combination of R¹ and Y corresponds in each case to one row of Table A (compounds I.A.254aA-1 to I.A.254aA-366)

### Table 255a

Compounds I.A in which Z is C(CH₂CH₂)(CH₂)₃CH₂, R², R³ and R⁴ are H, R⁵ is CH₂CH(CH₃)₂ and the combination of R¹ and Y corresponds in each case to one row of Table A (compounds I.A.255aA-1 to I.A.255aA-366)

### Table 256a

Compounds I.A in which Z is CH₂CH₂CH(CH₃)CH₂CH₂, R², R³ and R⁴ are H, R⁵ is CH₂CH(CH₃)₂ and the combination of R¹ and Y corresponds in each case to one row of Table A (compounds I.A.256aA-1 to I.A.256aA-366)

### Table 257a

Compounds I.A in which Z is CH₂CH₂CH₂CH(CH₃)CH₂, R², R³ and R⁴ are H, R⁵ is CH₂CH(CH₃)₂ and the combination of R¹ and Y corresponds in each case to one row of Table A (compounds I.A.257aA-1 to I.A.257aA-366)

### Table 258a

Compounds I.A in which Z is CH₂(CH₂)₃CH(CH₃), R², R³ and R⁴ are H, R⁵ is CH₂CH(CH₃)₂ and the combination of R¹ and Y corresponds in each case to one row of Table A (compounds I.A.258aA-1 to I.A.258aA-366)

### Table 259a

Compounds I.A in which Z is (E) CH═CHCH₂, R², R³ and R⁴ are H, R⁵ is CH₂CH(CH₃)₂ and the combination of R¹ and Y corresponds in each case to one row of Table A (compounds I.A.259aA-1 to I.A.259aA-366)

### Table 260a

Compounds I.A in which Z is (E) C(CH₃)═CHCH₂, R², R³ and R⁴ are H, R⁵ is CH₂CH(CH₃)₂ and the combination of R¹ and Y corresponds in each case to one row of Table A (compounds I.A.260aA-1 to I.A.260aA-366)

### Table 261a

Compounds I.A in which Z is (E) C(CH₃)═C(CH₃)CH₂, R², R³ and R⁴ are H, R⁵ is CH₂CH(CH₃)₂ and the combination of R¹ and Y corresponds in each case to one row of Table A (compounds I.A.261aA-1 to I.A.261aA-366)

### Table 262a

Compounds I.A in which Z is (E) CH═C(CH₃)CH₂, R², R³ and R⁴ are H, R⁵ is CH₂CH(CH₃)₂ and the combination of R¹ and Y corresponds in each case to one row of Table A (compounds I.A.262aA-1 to I.A.262aA-366)

### Table 263a

Compounds I.A in which Z is (E) CH₂CH═CHCH₂, R², R³ and R⁴ are H, R⁵ is CH₂CH(CH₃)₂ and the combination of R¹ and Y corresponds in each case to one row of Table A (compounds I.A.263aA-1 to I.A.263aA-366)

### Table 264a

Compounds I.A in which Z is (E) CH₂C(CH₃)═CHCH₂, R², R³ and R⁴ are H, R⁵ is CH₂CH(CH₃)₂ and the combination of R¹ and Y corresponds in each case to one row of Table A (compounds I.A.264aA-1 to I.A.264aA-366)

### Table 265a

Compounds I.A in which Z is (E) CH₂CH═C(CH₃)CH₂, R², R³ and R⁴ are H, R⁵ is CH₂CH(CH₃)₂ and the combination of R¹ and Y corresponds in each case to one row of Table A (compounds I.A.265aA-1 to I.A.265aA-366)

### Table 266a

Compounds I.A in which Z is (E) CH₂C(CH₃)═C(CH₃)CH₂, R², R³ and R⁴ are H, R⁵ is CH₂CH(CH₃)₂ and the combination of R¹ and Y corresponds in each case to one row of Table A (compounds I.A.266aA-1 to I.A.266aA-366)

### Table 267a

Compounds I.A in which Z is (E) C(CH₃)═C(CH₃)(CH₂)₂CH₂, R², R³ and R⁴ are H, R⁵ is CH₂CH(CH₃)₂ and the combination of R¹ and Y corresponds in each case to one row of Table A (compounds I.A.267aA-1 to I.A.267aA-366)

### Table 268a

Compounds I.A in which Z is (E) C(CH₃)═CH(CH₂)₂CH₂, R², R³ and R⁴ are H, R⁵ is CH₂CH(CH₃)₂ and the combination of R¹ and Y corresponds in each case to one row of Table A (compounds I.A.268aA-1 to I.A.268aA-366)

### Table 269a

Compounds I.A in which Z is (E) CH═C(CH₃)(CH₂)₂CH₂, R², R³ and R⁴ are H, R⁵ is CH₂CH(CH₃)₂ and the combination of R¹ and Y corresponds in each case to one row of Table A (compounds I.A.269aA-1 to I.A.269aA-366)

### Table 270a

Compounds I.A in which Z is CH₂C═CCH₂, R², R³ and R⁴ are H, R⁵ is CH₂CH(CH₃)₂ and the combination of R¹ and Y corresponds in each case to one row of Table A (compounds I.A.270aA-1 to I.A.270aA-366)

### Table 271a

Compounds I.A in which Z is CH₂CH₂CH₂, R², R³ and R⁴ are H, R⁵ is CH₂C(CH₃)₃ and the combination of R¹ and Y corresponds in each case to one row of Table A (compounds I.A.271aA-1 to I.A.271 aA-366)

### Table 272a

Compounds I.A in which Z is CH₂(CH₂)₂CH₂, R², R³ and R⁴ are H, R⁵ is CH₂C(CH₃)₃ and the combination of R¹ and Y corresponds in each case to one row of Table A (compounds I.A.272aA-1 to I.A.272aA-366)

### Table 273a

Compounds I.A in which Z is CH₂(CH₂)₃CH₂, R², R³ and R⁴ are H, R⁵ is CH₂C(CH₃)₃ and the combination of R¹ and Y corresponds in each case to one row of Table A (compounds I.A.273aA-1 to I.A.273aA-366)

### Table 274a

Compounds I.A in which Z is CH₂CH(CH₃)CH₂, R², R³ and R⁴ are H, R⁵ is CH₂C(CH₃)₃ and the combination of R¹ and Y corresponds in each case to one row of Table A (compounds I.A.274aA-1 to I.A.274aA-366)

### Table 275a

Compounds I.A in which Z is CH₂CH₂CH(CH₃), R², R³ and R⁴ are H, R⁵ is CH₂C(CH₃)₃ and the combination of R¹ and Y corresponds in each case to one row of Table A (compounds I.A.275aA-1 to I.A.275aA-366)

### Table 276a

Compounds I.A in which Z is CH(CH₃)CH₂CH₂, R², R³ and R⁴ are H, R⁵ is CH₂C(CH₃)₃ and the combination of R¹ and Y corresponds in each case to one row of Table A (compounds I.A.276aA-1 to I.A.276aA-366)

### Table 277a

Compounds I.A in which Z is CH₂C(CH₃)₂CH₂, R², R³ and R⁴ are H, R⁵ is CH₂C(CH₃)₃ and the combination of R¹ and Y corresponds in each case to one row of Table A (compounds I.A.277aA-1 to I.A.277aA-366)

### Table 278a

Compounds I.A in which Z is CH₂C(CH₂CH₂)CH₂, R², R³ and R⁴ are H, R⁵ is CH₂C(CH₃)₃ and the combination of R¹ and Y corresponds in each case to one row of Table A (compounds I.A.278aA-1 to I.A.278aA-366)

### Table 279a

Compounds I.A in which Z is C(CH₂CH₂)CH₂CH₂, R², R³ and R⁴ are H, R⁵ is CH₂C(CH₃)₃ and the combination of R¹ and Y corresponds in each case to one row of Table A (compounds I.A.279aA-1 to I.A.279aA-366)

### Table 280a

Compounds I.A in which Z is CH₂CH(CH₃)(CH₂)₂CH₂, R², R³ and R⁴ are H, R⁵ is CH₂C(CH₃)₃ and the combination of R¹ and Y corresponds in each case to one row of Table A (compounds I.A.280aA-1 to I.A.280aA-366)

### Table 281a

Compounds I.A in which Z is C(CH₃)₂(CH₂)₃CH₂, R², R³ and R⁴ are H, R⁵ is CH₂C(CH₃)₃ and the combination of R¹ and Y corresponds in each case to one row of Table A (compounds I.A.281aA-1 to I.A.281aA-366)

### Table 282a

Compounds I.A in which Z is C(CH₂CH₂)(CH₂)₃CH₂, R², R³ and R⁴ are H, R⁵ is CH₂C(CH₃)₃ and the combination of R¹ and Y corresponds in each case to one row of Table A (compounds I.A.282aA-1 to I.A.282aA-366)

### Table 283a

Compounds I.A in which Z is CH₂CH₂CH(CH₃)CH₂CH₂, R², R³ and R⁴ are H, R⁵ is CH₂C(CH₃)₃ and the combination of R¹ and Y corresponds in each case to one row of Table A (compounds I.A.283aA-1 to I.A.283aA-366)

### Table 284a

Compounds I.A in which Z is CH₂CH₂CH₂CH(CH₃)CH₂, R², R³ and R⁴ are H, R⁵ is CH₂C(CH₃)₃ and the combination of R¹ and Y corresponds in each case to one row of Table A (compounds I.A.284aA-1 to I.A.284aA-366)

### Table 285a

Compounds I.A in which Z is CH₂(CH₂)₃CH(CH₃), R², R³ and R⁴ are H, R⁵ is CH₂C(CH₃)₃ and the combination of R¹ and Y corresponds in each case to one row of Table A (compounds I.A.285aA-1 to I.A.285aA-366)

### Table 286a

Compounds I.A in which Z is (E) CH═CHCH₂, R², R³ and R⁴ are H, R⁵ is CH₂C(CH₃)₃ and the combination of R¹ and Y corresponds in each case to one row of Table A (compounds I.A.286aA-1 to I.A.286aA-366)

### Table 287a

Compounds I.A in which Z is (E) C(CH₃)═CHCH₂, R², R³ and R⁴ are H, R⁵ is CH₂C(CH₃)₃ and the combination of R¹ and Y corresponds in each case to one row of Table A (compounds I.A.287aA-1 to I.A.287aA-366)

### Table 288a

Compounds I.A in which Z is (E) C(CH₃)═C(CH₃)CH₂, R², R³ and R⁴ are H, R⁵ is CH₂C(CH₃)₃ and the combination of R¹ and Y corresponds in each case to one row of Table A (compounds I.A.288aA-1 to I.A.288aA-366)

### Table 289a

Compounds I.A in which Z is (E) CH═C(CH₃)CH₂, R², R³ and R⁴ are H, R⁵ is CH₂C(CH₃)₃ and the combination of R¹ and Y corresponds in each case to one row of Table A (compounds I.A.289aA-1 to I.A.289aA-366)

### Table 290a

Compounds I.A in which Z is (E) CH₂CH═CHCH₂, R², R³ and R⁴ are H, R⁵ is CH₂C(CH₃)₃ and the combination of R¹ and Y corresponds in each case to one row of Table A (compounds I.A.290aA-1 to I.A.290aA-366)

### Table 291a

Compounds I.A in which Z is (E) CH₂C(CH₃)═CHCH₂, R², R³ and R⁴ are H, R⁵ is CH₂C(CH₃)₃ and the combination of R¹ and Y corresponds in each case to one row of Table A (compounds I.A.291aA-1 to I.A.291aA-366)

### Table 292a

Compounds I.A in which Z is (E) CH₂CH═C(CH₃)CH₂, R², R³ and R⁴ are H, R⁵ is CH₂C(CH₃)₃ and the combination of R¹ and Y corresponds in each case to one row of Table A (compounds I.A.292aA-1 to I.A.292aA-366)

### Table 293a

Compounds I.A in which Z is (E) CH₂C(CH₃)═C(CH₃)CH₂, R², R³ and R⁴ are H, R⁵ is CH₂C(CH₃)₃ and the combination of R¹ and Y corresponds in each case to one row of Table A (compounds I.A.293aA-1 to I.A.293aA-366)

### Table 294a

Compounds I.A in which Z is (E) C(CH₃)═C(CH₃)(CH₂)₂CH₂, R², R³ and R⁴ are H, R⁵ is CH₂C(CH₃)₃ and the combination of R¹ and Y corresponds in each case to one row of Table A (compounds I.A.294aA-1 to I.A.294aA-366)

### Table 295a

Compounds I.A in which Z is (E) C(CH₃)═CH(CH₂)₂CH₂, R², R³ and R⁴ are H, R⁵ is CH₂C(CH₃)₃ and the combination of R¹ and Y corresponds in each case to one row of Table A (compounds I.A.295aA-1 to I.A.295aA-366)

### Table 296a

Compounds I.A in which Z is (E) CH═C(CH₃)(CH₂)₂CH₂, R², R³ and R⁴ are H, R⁵ is CH₂C(CH₃)₃ and the combination of R¹ and Y corresponds in each case to one row of Table A (compounds I.A.296aA-1 to I.A.296aA-366)

### Table 297a

Compounds I.A in which Z is CH₂C≡CCH₂, R², R³ and R⁴ are H, R⁵ is CH₂C(CH₃)₃ and the combination of R¹ and Y corresponds in each case to one row of Table A (compounds I.A.297aA-1 to I.A.297aA-366)

### Table 298a

Compounds I.A in which Z is CH₂CH₂CH₂, R², R³ and R⁴ are H, R⁵ is CH(CH₃)CH[CH₂CH₂] and the combination of R¹ and Y corresponds in each case to one row of Table A (compounds I.A.298aA-1 to I.A.298aA-366)

### Table 299a

Compounds I.A in which Z is CH₂(CH₂)₂CH₂, R², R³ and R⁴ are H, R⁵ is CH(CH₃)CH[CH₂CH₂] and the combination of R¹ and Y corresponds in each case to one row of Table A (compounds I.A.299aA-1 to I.A.299aA-366)

### Table 300a

Compounds I.A in which Z is CH₂(CH₂)₃CH₂, R², R³ and R⁴ are H, R⁵ is CH(CH₃)CH[CH₂CH₂] and the combination of R¹ and Y corresponds in each case to one row of Table A (compounds I.A.300aA-1 to I.A.300aA-366)

### Table 301a

Compounds I.A in which Z is CH₂CH(CH₃)CH₂, R², R³ and R⁴ are H, R⁵ is CH(CH₃)CH[CH₂CH₂] and the combination of R¹ and Y corresponds in each case to one row of Table A (compounds I.A.301aA-1 to I.A.301aA-366)

### Table 302a

Compounds I.A in which Z is CH₂CH₂CH(CH₃), R², R³ and R⁴ are H, R⁵ is CH(CH₃)CH[CH₂CH₂] and the combination of R¹ and Y corresponds in each case to one row of Table A (compounds I.A.302aA-1 to I.A.302aA-366)

### Table 303a

Compounds I.A in which Z is CH(CH₃)CH₂CH₂, R², R³ and R⁴ are H, R⁵ is CH(CH₃)CH[CH₂CH₂] and the combination of R¹ and Y corresponds in each case to one row of Table A (compounds I.A.303aA-1 to I.A.303aA-366)

### Table 304a

Compounds I.A in which Z is CH₂C(CH₃)₂CH₂, R², R³ and R⁴ are H, R⁵ is CH(CH₃)CH[CH₂CH₂] and the combination of R¹ and Y corresponds in each case to one row of Table A (compounds I.A.304aA-1 to I.A.304aA-366)

### Table 305a

Compounds I.A in which Z is CH₂C(CH₂CH₂)CH₂, R², R³ and R⁴ are H, R⁵ is CH(CH₃)CH[CH₂CH_{2]} and the combination of R¹ and Y corresponds in each case to one row of Table A (compounds I.A.305aA-1 to I.A.305aA-366)

### Table 306a

Compounds I.A in which Z is C(CH₂CH₂)CH₂CH₂, R², R³ and R⁴ are H, R⁵ is CH(CH₃)CH[CH₂CH₂] and the combination of R¹ and Y corresponds in each case to one row of Table A (compounds I.A.306aA-1 to I.A.306aA-366)

### Table 307a

Compounds I.A in which Z is CH₂CH(CH₃)(CH₂)₂CH₂, R², R³ and R⁴ are H, R⁵ is CH(CH₃)CH[CH₂CH₂] and the combination of R¹ and Y corresponds in each case to one row of Table A (compounds I.A.307aA-1 to I.A.307aA-366)

### Table 308a

Compounds I.A in which Z is C(CH₃)₂(CH₂)₃CH₂, R², R³ and R⁴ are H, R⁵ is CH(CH₃)CH[CH₂CH_{2]} and the combination of R¹ and Y corresponds in each case to one row of Table A (compounds I.A.308aA-1 to I.A.308aA-366)

### Table 309a

Compounds I.A in which Z is C(CH₂CH₂)(CH₂)₃CH₂, R², R³ and R⁴ are H, R⁵ is CH(CH₃)CH[CH₂CH_{2]} and the combination of R¹ and Y corresponds in each case to one row of Table A (compounds I.A.309aA-1 to I.A.309aA-366)

### Table 310a

Compounds I.A in which Z is CH₂CH₂CH(CH₃)CH₂CH₂, R², R³ and R⁴ are H, R⁵ is CH(CH₃)CH[CH₂CH₂] and the combination of R¹ and Y corresponds in each case to one row of Table A (compounds I.A.310aA-1 to I.A.310aA-366)

### Table 311a

Compounds I.A in which Z is CH₂CH₂CH₂CH(CH₃)CH₂, R², R³ and R⁴ are H, R⁵ is CH(CH₃)CH[CH₂CH_{2]} and the combination of R¹ and Y corresponds in each case to one row of Table A (compounds I.A.311aA-1 to I.A.311aA-366)

### Table 312a

Compounds I.A in which Z is CH₂(CH₂)₃CH(CH₃), R², R³ and R⁴ are H, R⁵ is CH(CH₃)CH[CH₂CH_{2]} and the combination of R¹ and Y corresponds in each case to one row of Table A (compounds I.A.312aA-1 to I.A.312aA-366)

### Table 313a

Compounds I.A in which Z is (E) CH═CHCH₂, R², R³ and R⁴ are H, R⁵ is CH(CH₃)CH[CH₂CH₂] and the combination of R¹ and Y corresponds in each case to one row of Table A (compounds I.A.313aA-1 to I.A.313aA-366)

### Table 314a

Compounds I.A in which Z is (E) C(CH₃)═CHCH₂, R², R³ and R⁴ are H, R⁵ is CH(CH₃)CH[CH₂CH₂] and the combination of R¹ and Y corresponds in each case to one row of Table A (compounds I.A.314aA-1 to I.A.314aA-366)

### Table 315a

Compounds I.A in which Z is (E) C(CH₃)═C(CH₃)CH₂, R², R³ and R⁴ are H, R⁵ is CH(CH₃)CH[CH₂CH₂] and the combination of R¹ and Y corresponds in each case to one row of Table A (compounds I.A.315aA-1 to I.A.315aA-366)

### Table 316a

Compounds I.A in which Z is (E) CH═C(CH₃)CH₂, R², R³ and R⁴ are H, R⁵ is CH(CH₃)CH[CH₂CH₂] and the combination of R¹ and Y corresponds in each case to one row of Table A (compounds I.A.316aA-1 to I.A.316aA-366)

### Table 317a

Compounds I.A in which Z is (E) CH₂CH═CHCH₂, R², R³ and R⁴ are H, R⁵ is CH(CH₃)CH[CH₂CH₂] and the combination of R¹ and Y corresponds in each case to one row of Table A (compounds I.A.317aA-1 to I.A.317aA-366)

### Table 318a

Compounds I.A in which Z is (E) CH₂C(CH₃)═CHCH₂, R², R³ and R⁴ are H, R⁵ is CH(CH₃)CH[CH₂CH₂] and the combination of R¹ and Y corresponds in each case to one row of Table A (compounds I.A.318aA-1 to I.A.318aA-366)

### Table 319a

Compounds I.A in which Z is (E) CH₂CH═C(CH₃)CH₂, R², R³ and R⁴ are H, R⁵ is CH(CH₃)CH[CH₂CH₂] and the combination of R¹ and Y corresponds in each case to one row of Table A (compounds I.A.319aA-1 to I.A.319aA-366)

### Table 320a

Compounds I.A in which Z is (E) CH₂C(CH₃)═C(CH₃)CH₂, R², R³ and R⁴ are H, R⁵ is CH(CH₃)CH[CH₂CH₂] and the combination of R¹ and Y corresponds in each case to one row of Table A (compounds I.A.320aA-1 to I.A.320aA-366)

### Table 321a

Compounds I.A in which Z is (E) C(CH₃)═C(CH₃)(CH₂)₂CH₂, R², R³ and R⁴ are H, R⁵ is CH(CH₃)CH[CH₂CH₂] and the combination of R¹ and Y corresponds in each case to one row of Table A (compounds I.A.321aA-1 to I.A.321aA-366)

### Table 322a

Compounds I.A in which Z is (E) C(CH₃)═CH(CH₂)₂CH₂, R², R³ and R⁴ are H, R⁵ is CH(CH₃)CH[CH₂CH₂] and the combination of R¹ and Y corresponds in each case to one row of Table A (compounds I.A.322aA-1 to I.A.322aA-366)

### Table 323a

Compounds I.A in which Z is (E) CH═C(CH₃)(CH₂)₂CH₂, R², R³ and R⁴ are H, R⁵ is CH(CH₃)CH[CH₂CH₂] and the combination of R¹ and Y corresponds in each case to one row of Table A (compounds I.A.323aA-1 to I.A.323aA-366)

### Table 324a

Compounds I.A in which Z is CH₂C≡CCH₂, R², R³ and R⁴ are H, R⁵ is CH(CH₃)CH[CH₂CH₂] and the combination of R¹ and Y corresponds in each case to one row of Table A (compounds I.A.324aA-1 to I.A.324aA-366)

### Table 325a

Compounds I.A in which Z is CH₂CH₂CH₂, R², R³ and R⁴ are H, R⁵ is C(CH₃)₃ and the combination of R¹ and Y corresponds in each case to one row of Table A (compounds I.A.325aA-1 to I.A.325aA-366)

### Table 326a

Compounds I.A in which Z is CH₂(CH₂)₂CH₂, R², R³ and R⁴ are H, R⁵ is C(CH₃)₃ and the combination of R¹ and Y corresponds in each case to one row of Table A (compounds I.A.326aA-1 to I.A.326aA-366)

### Table 327a

Compounds I.A in which Z is CH₂(CH₂)₃CH₂, R², R³ and R⁴ are H, R⁵ is C(CH₃)₃ and the combination of R¹ and Y corresponds in each case to one row of Table A (compounds I.A.327aA-1 to I.A.327aA-366)

### Table 328a

Compounds I.A in which Z is CH₂CH(CH₃)CH₂, R², R³ and R⁴ are H, R⁵ is C(CH₃)₃ and the combination of R¹ and Y corresponds in each case to one row of Table A (compounds I.A.328aA-1 to I.A.328aA-366)

### Table 329a

Compounds I.A in which Z is CH₂CH₂CH(CH₃), R², R³ and R⁴ are H, R⁵ is C(CH₃)₃ and the combination of R¹ and Y corresponds in each case to one row of Table A (compounds I.A.329aA-1 to I.A.329aA-366)

### Table 330a

Compounds I.A in which Z is CH(CH₃)CH₂CH₂, R², R³ and R⁴ are H, R⁵ is C(CH₃)₃ and the combination of R¹ and Y corresponds in each case to one row of Table A (compounds I.A.330aA-1 to I.A.330aA-366)

### Table 331a

Compounds I.A in which Z is CH₂C(CH₃)₂CH₂, R², R³ and R⁴ are H, R⁵ is C(CH₃)₃ and the combination of R¹ and Y corresponds in each case to one row of Table A (compounds I.A.331aA-1 to I.A.331aA-366)

### Table 332a

Compounds I.A in which Z is CH₂C(CH₂CH₂)CH₂, R², R³ and R⁴ are H, R⁵ is C(CH₃)₃ and the combination of R¹ and Y corresponds in each case to one row of Table A (compounds I.A.332aA-1 to I.A.332aA-366)

### Table 333a

Compounds I.A in which Z is C(CH₂CH₂)CH₂CH₂, R², R³ and R⁴ are H, R⁵ is C(CH₃)₃ and the combination of R¹ and Y corresponds in each case to one row of Table A (compounds I.A.333aA-1 to I.A.333aA-366)

### Table 334a

Compounds I.A in which Z is CH₂CH(CH₃)(CH₂)₂CH₂, R², R³ and R⁴ are H, R⁵ is C(CH₃)₃ and the combination of R¹ and Y corresponds in each case to one row of Table A (compounds I.A.334aA-1 to I.A.334aA-366)

### Table 335a

Compounds I.A in which Z is C(CH₃)₂(CH₂)₃CH₂, R², R³ and R⁴ are H, R⁵ is C(CH₃)₃ and the combination of R¹ and Y corresponds in each case to one row of Table A (compounds I.A.335aA-1 to I.A.335aA-366)

### Table 336a

Compounds I.A in which Z is C(CH₂CH₂)(CH₂)₃CH₂, R², R³ and R⁴ are H, R⁵ is C(CH₃)₃ and the combination of R¹ and Y corresponds in each case to one row of Table A (compounds I.A.336aA-1 to I.A.336aA-366)

### Table 337a

Compounds I.A in which Z is CH₂CH₂CH(CH₃)CH₂CH₂, R², R³ and R⁴ are H, R⁵ is C(CH₃)₃ and the combination of R¹ and Y corresponds in each case to one row of Table A (compounds I.A.337aA-1 to I.A.337aA-366)

### Table 338a

Compounds I.A in which Z is CH₂CH₂CH₂CH(CH₃)CH₂, R², R³ and R⁴ are H, R⁵ is C(CH₃)₃ and the combination of R¹ and Y corresponds in each case to one row of Table A (compounds I.A.338aA-1 to I.A.338aA-366)

### Table 339a

Compounds I.A in which Z is CH₂(CH₂)₃CH(CH₃), R², R³ and R⁴ are H, R⁵ is C(CH₃)₃ and the combination of R¹ and Y corresponds in each case to one row of Table A (compounds I.A.339aA-1 to I.A.339aA-366)

### Table 340a

Compounds I.A in which Z is (E) CH═CHCH₂, R², R³ and R⁴ are H, R⁵ is C(CH₃)₃ and the combination of R¹ and Y corresponds in each case to one row of Table A (compounds I.A.340aA-1 to I.A.340aA-366)

### Table 341a

Compounds I.A in which Z is (E) C(CH₃)═CHCH₂, R², R³ and R⁴ are H, R⁵ is C(CH₃)₃ and the combination of R¹ and Y corresponds in each case to one row of Table A (compounds I.A.261aA-1 to I.A.261aA-366)

### Table 342a

Compounds I.A in which Z is (E) C(CH₃)═C(CH₃)CH₂, R², R³ and R⁴ are H, R⁵ is C(CH₃)₃ and the combination of R¹ and Y corresponds in each case to one row of Table A (compounds I.A.342aA-1 to I.A.342aA-366)

### Table 343a

Compounds I.A in which Z is (E) CH═C(CH₃)CH₂, R², R³ and R⁴ are H, R⁵ is C(CH₃)₃ and the combination of R¹ and Y corresponds in each case to one row of Table A (compounds I.A.343aA-1 to I.A.343aA-366)

### Table 344a

Compounds I.A in which Z is (E) CH₂CH═CHCH₂, R², R³ and R⁴ are H, R⁵ is C(CH₃)₃ and the combination of R¹ and Y corresponds in each case to one row of Table A (compounds I.A.344aA-1 to I.A.344aA-366)

### Table 345a

Compounds I.A in which Z is (E) CH₂C(CH₃)═CHCH₂, R², R³ and R⁴ are H, R⁵ is C(CH₃)₃ and the combination of R¹ and Y corresponds in each case to one row of Table A (compounds I.A.345aA-1 to I.A.345aA-366)

### Table 346a

Compounds I.A in which Z is (E) CH₂CH═C(CH₃)CH₂, R², R³ and R⁴ are H, R⁵ is C(CH₃)₃ and the combination of R¹ and Y corresponds in each case to one row of Table A (compounds I.A.346aA-1 to I.A.346aA-366)

### Table 347a

Compounds I.A in which Z is (E) CH₂C(CH₃)═C(CH₃)CH₂, R², R³ and R⁴ are H, R⁵ is C(CH₃)₃ and the combination of R¹ and Y corresponds in each case to one row of Table A (compounds I.A.347aA-1 to I.A.347aA-366)

### Table 348a

Compounds I.A in which Z is (E) C(CH₃)═C(CH₃)(CH₂)₂CH₂, R², R³ and R⁴ are H, R⁵ is C(CH₃)₃ and the combination of R¹ and Y corresponds in each case to one row of Table A (compounds I.A.348aA-1 to I.A.348aA-366)

### Table 349a

Compounds I.A in which Z is (E) C(CH₃)═CH(CH₂)₂CH₂, R², R³ and R⁴ are H, R⁵ is C(CH₃)₃ and the combination of R¹ and Y corresponds in each case to one row of Table A (compounds I.A.349aA-1 to I.A.349aA-366)

### Table 350a

Compounds I.A in which Z is (E) CH═C(CH₃)(CH₂)₂CH₂, R², R³ and R⁴ are H, R⁵ is C(CH₃)₃ and the combination of R¹ and Y corresponds in each case to one row of Table A (compounds I.A.350aA-1 to I.A.350aA-366)

### Table 351a

Compounds I.A in which Z is CH₂C≡CCH₂, R², R³ and R⁴ are H, R⁵ is C(CH₃)₃ and the combination of R¹ and Y corresponds in each case to one row of Table A (compounds I.A.351aA-1 to I.A.351aA-366)

TABLE A

| Row | R¹ | Y |
|---|---|---|
| A-1 | pyridin-2-yl | O |
| A-2 | pyridin-3-yl | O |
| A-3 | pyridin-4-yl | O |
| A-4 | 3-chloropyridin-2-yl | O |
| A-5 | 4-chloropyridin-2-yl | O |
| A-6 | 5-chloropyridin-2-yl | O |
| A-7 | 6-chloropyridin-2-yl | O |
| A-8 | 2-chloropyridin-3-yl | O |
| A-9 | 4-chloropyridin-3-yl | O |
| A-10 | 5-chloropyridin-3-yl | O |
| A-11 | 6-chloropyridin-3-yl | O |
| A-12 | 2-chloropyridin-4-yl | O |
| A-13 | 3-chloropyridin-4-yl | O |
| A-14 | 4-chloropyridin-5-yl | O |
| A-15 | 3-fluoropyridin-2-yl | O |
| A-16 | 4-fluoropyridin-2-yl | O |
| A-17 | 5-fluoropyridin-2-yl | O |
| A-18 | 6-fluoropyridin-2-yl | O |
| A-19 | 2-fluoropyridin-3-yl | O |
| A-20 | 4-fluoropyridin-3-yl | O |
| A-21 | 5-fluoropyridin-3-yl | O |
| A-22 | 6-fluoropyridin-3-yl | O |
| A-23 | 2-fluoropyridin-4-yl | O |
| A-24 | 3-fluoropyridin-4-yl | O |
| A-25 | 3-methylpyridin-2-yl | O |
| A-26 | 4-methylpyridin-2-yl | O |
| A-27 | 5-methylpyridin-2-yl | O |
| A-28 | 6-methylpyridin-2-yl | O |
| A-29 | 2-methylpyridin-3-yl | O |
| A-30 | 4-methylpyridin-3-yl | O |
| A-31 | 5-methylpyridin-3-yl | O |
| A-32 | 6-methylpyridin-3-yl | O |
| A-33 | 2-methylpyridin-4-yl | O |
| A-34 | 3-methylpyridin-4-yl | O |
| A-35 | 4-methylpyridin-5-yl | O |
| A-36 | 3-methoxypyridin-2-yl | O |
| A-37 | 4-methoxypyridin-2-yl | O |
| A-38 | 5-methoxypyridin-2-yl | O |
| A-39 | 6-methoxypyridin-2-yl | O |
| A-40 | 2-methoxypyridin-3-yl | O |
| A-41 | 4-methoxypyridin-3-yl | O |
| A-42 | 5-methoxypyridin-3-yl | O |
| A-43 | 6-methoxypyridin-3-yl | O |
| A-44 | 2-methoxypyridin-4-yl | O |
| A-45 | 3-methoxypyridin-4-yl | O |
| A-46 | 3,5-dichloropyridin-2-yl | O |
| A-47 | 2,4-dichloropyridin-3-yl | O |
| A-48 | 2,5-dichloropyridin-3-yl | O |
| A-49 | 2,6-dichloropyridin-3-yl | O |
| A-50 | 2,6-dichloropyridin-4-yl | O |
| A-51 | 3,5-dichloropyridin-4-yl | O |
| A-52 | 3,6-dichloropyridin-4-yl | O |
| A-53 | 2,5-dichloropyridin-4-yl | O |
| A-54 | 2,3-dichloropyridin-4-yl | O |
| A-55 | 2,4-dichloropyridin-5-yl | O |
| A-56 | 3,5-difluoropyridin-2-yl | O |
| A-57 | 3,5-difluoropyridin-4-yl | O |
| A-58 | 2,3-difluoropyridin-4-yl | O |
| A-59 | 3,5-dimethylpyridin-2-yl | O |
| A-60 | 3,4-dimethylpyridin-2-yl | O |
| A-61 | 4,6-dimethylpyridin-3-yl | O |
| A-62 | 2,4-dimethylpyridin-3-yl | O |
| A-63 | 3,5-dimethylpyridin-4-yl | O |
| A-64 | 2-chloro-3-fluoropyridin-4-yl | O |
| A-65 | 4-chloro-3-methylpyridin-2-yl | O |
| A-66 | 5-chloro-3-methylpyridin-2-yl | O |
| A-67 | 5-fluoro-3-methylpyridin-2-yl | O |
| A-68 | 3-chloro-5-trifluoropyridin-2-yl | O |
| A-69 | 2-chloro-6-trifluoropyridin-3-yl | O |
| A-70 | 2,4-dichloro-6-methylpyridin-3-yl | O |
| A-71 | 3,4,5-trichloropyridin-2-yl | O |
| A-72 | 2,4,6-trichloropyridin-3-yl | O |
| A-73 | pyrimidin-2-yl | O |
| A-74 | pyrimidin-5-yl | O |
| A-75 | 6-chloropyrimidin-3-yl | O |
| A-76 | 6-methylpyrimidin-3-yl | O |
| A-77 | 6-methoxypyrimidin-3-yl | O |
| A-78 | 2,4-dichloropyrimidin-3-yl | O |
| A-79 | 2,6-dichloropyrimidin-3-yl | O |
| A-80 | 2,4-difluoropyrimidin-3-yl | O |
| A-81 | 2,6-difluoropyrimidin-3-yl | O |
| A-82 | 1,3,5-triazin-2-yl | O |
| A-83 | thien-2-yl | O |
| A-84 | thien-3-yl | O |
| A-85 | 5-chlorothien-2-yl | O |
| A-86 | 2-chlorothien-3-yl | O |
| A-87 | 4-chlorothien-3-yl | O |
| A-88 | 5-chlorothien-3-yl | O |
| A-89 | 2-bromothien-3-yl | O |
| A-90 | 4-bromothien-3-yl | O |
| A-91 | 5-bromothien-3-yl | O |
| A-92 | 5-methylthien-2-yl | O |
| A-93 | 2-methylthien-4-yl | O |
| A-94 | 4,5-dichlorothien-2-yl | O |
| A-95 | 2,5-dichlorothien-3-yl | O |
| A-96 | 2,3-dichlorothien-4-yl | O |
| A-97 | 2,5-dibromothien-3-yl | O |
| A-98 | 4,5-dimethylthien-2-yl | O |
| A-99 | 2,5-dimethylthien-3-yl | O |
| A-100 | 2,3-dimethylthien-4-yl | O |
| A-101 | 3,4,5-trichlorothien-2-yl | O |
| A-102 | 2,4,5-trichlorothien-3-yl | O |
| A-103 | 2,4,5-tribromothien-3-yl | O |
| A-104 | 3,4,5-trimethylthien-2-yl | O |
| A-105 | 2-furyl | O |
| A-106 | 4-chlorofur-2-yl | O |
| A-107 | 5-chlorofur-2-yl | O |
| A-108 | 3-bromofur-2-yl | O |
| A-109 | 4-bromofur-2-yl | O |
| A-110 | 5-bromofur-2-yl | O |
| A-111 | 3,4-dichlorofur-2-yl | O |
| A-112 | 4,5-dibromofur-2-yl | O |
| A-113 | thiazol-2-yl | O |
| A-114 | thiazol-4-yl | O |
| A-115 | thiazol-5-yl | O |
| A-116 | isothiazol-3-yl | O |
| A-117 | isothiazol-4-yl | O |
| A-118 | isothiazol-5-yl | O |
| A-119 | 2-chlorothiazol-4-yl | O |
| A-120 | 2-chlorothiazol-5-yl | O |
| A-121 | 4-chlorothiazol-5-yl | O |
| A-122 | 2-bromothiazol-4-yl | O |
| A-123 | 2-bromothiazol-5-yl | O |
| A-124 | 2,4-dichlorothiazol-5-yl | O |
| A-125 | 3-chloroisothiazol-4-yl | O |
| A-126 | 5-methylisothiazol-3-yl | O |
| A-127 | 3-methylisothiazol-4-yl | O |
| A-128 | 3-methylisothiazol-5-yl | O |
| A-129 | 4,5-dichloroisothiazol-3-yl | O |
| A-130 | 4,5-dimethylisothiazol-3-yl | O |
| A-131 | 3,5-dimethylisothiazol-4-yl | O |
| A-132 | 3,4-dichloroisothiazol-5-yl | O |
| A-133 | oxazol-2-yl | O |
| A-134 | oxazol-4-yl | O |
| A-135 | oxazol-5-yl | O |
| A-136 | isoxazol-3-yl | O |
| A-137 | isoxazol-4-yl | O |
| A-138 | isoxazol-5-yl | O |
| A-139 | 3-chloroisoxazol-5-yl | O |
| A-140 | 5-methylisoxazol-3-yl | O |
| A-141 | 5-methylisoxazol-4-yl | O |
| A-142 | 3-methylisoxazol-5-yl | O |
| A-143 | 3,5-dimethylisoxazol-4-yl | O |
| A-144 | 3-chloro-5-methylisoxazol-4-yl | O |
| A-145 | 3-methyl-4-chloroisoxazol-5-yl | O |
| A-146 | 1-methylpyrazol-3-yl | O |
| A-147 | 1-methylpyrazol-4-yl | O |
| A-148 | 1-methylpyrazol-5-yl | O |
| A-149 | 1,3-dimethylpyrazol-4-yl | O |
| A-150 | 1,5-dimethylpyrazol-4-yl | O |
| A-151 | 1,3,5-trimethylpyrazol-4-yl | O |
| A-152 | 1-methylimidazol-4-yl | O |
| A-153 | 1,5-dimethylimidazol-4-yl | O |
| A-154 | 1,2-dimethylimidazol-5-yl | O |
| A-155 | 1,4-dimethylimidazol-5-yl | O |
| A-156 | pyridin-2-yl | — [*] |
| A-157 | pyridin-3-yl | — [*] |
| A-158 | pyridin-4-yl | — [*] |
| A-159 | 3-chloropyridin-2-yl | — [*] |
| A-160 | 4-chloropyridin-2-yl | — [*] |
| A-161 | 5-chloropyridin-2-yl | — [*] |
| A-162 | 6-chloropyridin-2-yl | — [*] |
| A-163 | 2-chloropyridin-3-yl | — [*] |
| A-164 | 4-chloropyridin-3-yl | — [*] |
| A-165 | 5-chloropyridin-3-yl | — [*] |
| A-166 | 6-chloropyridin-3-yl | — [*] |
| A-167 | 2-chloropyridin-4-yl | — [*] |
| A-168 | 3-chloropyridin-4-yl | — [*] |
| A-169 | 4-chloropyridin-5-yl | — [*] |
| A-170 | 3-fluoropyridin-2-yl | — [*] |
| A-171 | 4-fluoropyridin-2-yl | — [*] |
| A-172 | 5-fluoropyridin-2-yl | — [*] |
| A-173 | 6-fluoropyridin-2-yl | — [*] |
| A-174 | 2-fluoropyridin-3-yl | — [*] |
| A-175 | 4-fluoropyridin-3-yl | — [*] |
| A-176 | 5-fluoropyridin-3-yl | — [*] |
| A-177 | 6-fluoropyridin-3-yl | — [*] |
| A-178 | 2-fluoropyridin-4-yl | — [*] |
| A-179 | 3-fluoropyridin-4-yl | — [*] |
| A-180 | 3-methylpyridin-2-yl | — [*] |
| A-181 | 4-methylpyridin-2-yl | — [*] |
| A-182 | 5-methylpyridin-2-yl | — [*] |
| A-183 | 6-methylpyridin-2-yl | — [*] |
| A-184 | 2-methylpyridin-3-yl | — [*] |
| A-185 | 4-methylpyridin-3-yl | — [*] |
| A-186 | 5-methylpyridin-3-yl | — [*] |
| A-187 | 6-methylpyridin-3-yl | — [*] |
| A-188 | 2-methylpyridin-4-yl | — [*] |
| A-189 | 3-methylpyridin-4-yl | — [*] |
| A-190 | 4-methylpyridin-5-yl | — [*] |
| A-191 | 3-methoxypyridin-2-yl | — [*] |
| A-192 | 4-methoxypyridin-2-yl | — [*] |
| A-193 | 5-methoxypyridin-2-yl | — [*] |
| A-194 | 6-methoxypyridin-2-yl | — [*] |
| A-195 | 2-methoxypyridin-3-yl | — [*] |
| A-196 | 4-methoxypyridin-3-yl | — [*] |
| A-197 | 5-methoxypyridin-3-yl | — [*] |
| A-198 | 6-methoxypyridin-3-yl | — [*] |
| A-199 | 2-methoxypyridin-4-yl | — [*] |
| A-200 | 3-methoxypyridin-4-yl | — [*] |
| A-201 | 3,5-dichloropyridin-2-yl | — [*] |
| A-202 | 2,4-dichloropyridin-3-yl | — [*] |
| A-203 | 2,5-dichloropyridin-3-yl | — [*] |
| A-204 | 2,6-dichloropyridin-3-yl | — [*] |
| A-205 | 2,6-dichloropyridin-4-yl | — [*] |
| A-206 | 3,5-dichloropyridin-4-yl | — [*] |
| A-207 | 3,6-dichloropyridin-4-yl | — [*] |
| A-208 | 2,5-dichloropyridin-4-yl | — [*] |
| A-209 | 2,3-dichloropyridin-4-yl | — [*] |
| A-210 | 2,4-dichloropyridin-5-yl | — [*] |
| A-211 | 3,5-difluoropyridin-2-yl | — [*] |
| A-212 | 3,5-difluoropyridin-4-yl | — [*] |
| A-213 | 2,3-difluoropyridin-4-yl | — [*] |
| A-214 | 3,5-dimethylpyridin-2-yl | — [*] |
| A-215 | 3,4-dimethylpyridin-2-yl | — [*] |
| A-216 | 4,6-dimethylpyridin-3-yl | — [*] |
| A-217 | 2,4-dimethylpyridin-3-yl | — [*] |
| A-218 | 3,5-dimethylpyridin-4-yl | — [*] |
| A-219 | 2-chloro-3-fluoropyridin-4-yl | — [*] |
| A-220 | 4-chloro-3-methylpyridin-2-yl | — [*] |
| A-221 | 5-chloro-3-methylpyridin-2-yl | — [*] |
| A-222 | 5-fluoro-3-methylpyridin-2-yl | — [*] |
| A-223 | 3-chloro-5-trifluoromethylpyridin-2- | — [*] |
| | yl | |
| A-224 | 2-chloro-6-trifluoromethylpyridin-3- | — [*] |
| | yl | |
| A-225 | 2,4-dichloro-6-methylpyridin-3-yl | — [*] |
| A-226 | 3,4,5-trichloropyridin-2-yl | — [*] |
| A-227 | 2,4,6-trichloropyridin-3-yl | — [*] |
| A-228 | pyrimidin-2-yl | — [*] |
| A-229 | pyrimidin-5-yl | — [*] |
| A-230 | 6-chloropyrimidin-3-yl | — [*] |
| A-231 | 6-methylpyrimidin-3-yl | — [*] |
| A-232 | 6-methoxypyrimidin-3-yl | — [*] |
| A-233 | 2,4-dichloropyrimidin-3-yl | — [*] |
| A-234 | 2,6-dichloropyrimidin-3-yl | — [*] |
| A-235 | 2,4-difluoropyrimidin-3-yl | — [*] |
| A-236 | 2,6-difluoropyrimidin-3-yl | — [*] |
| A-237 | 1,3,5-triazin-2-yl | — [*] |
| A-238 | thien-2-yl | — [*] |
| A-239 | thien-3-yl | — [*] |
| A-240 | 5-chlorothien-2-yl | — [*] |
| A-241 | 2-chlorothien-3-yl | — [*] |
| A-242 | 4-chlorothien-3-yl | — [*] |
| A-243 | 5-chlorothien-3-yl | — [*] |
| A-244 | 2-bromothien-3-yl | — [*] |
| A-245 | 4-bromothien-3-yl | — [*] |
| A-246 | 5-bromothien-3-yl | — [*] |
| A-247 | 5-methylthien-2-yl | — [*] |
| A-248 | 2-methylthien-4-yl | — [*] |
| A-249 | 4,5-dichlorothien-2-yl | — [*] |
| A-250 | 2,5-dichlorothien-3-yl | — [*] |
| A-251 | 2,3-dichlorothien-4-yl | — [*] |
| A-252 | 2,5-dibromothien-3-yl | — [*] |
| A-253 | 4,5-dimethylthien-2-yl | — [*] |
| A-254 | 2,5-dimethylthien-3-yl | — [*] |
| A-255 | 2,3-dimethylthien-4-yl | — [*] |
| A-256 | 3,4,5-trichlorothien-2-yl | — [*] |
| A-257 | 2,4,5-trichlorothien-3-yl | — [*] |
| A-258 | 2,4,5-tribromothien-3-yl | — [*] |
| A-259 | 3,4,5-trimethylthien-2-yl | — [*] |
| A-260 | 2-furyl | — [*] |
| A-261 | 4-chlorofur-2-yl | — [*] |
| A-262 | 5-chlorofur-2-yl | — [*] |
| A-263 | 3-bromofur-2-yl | — [*] |
| A-264 | 4-bromofur-2-yl | — [*] |
| A-265 | 5-bromofur-2-yl | — [*] |
| A-266 | 3,4-dichlorofur-2-yl | — [*] |
| A-267 | 4,5-dibromofur-2-yl | — [*] |
| A-268 | thiazol-2-yl | — [*] |
| A-269 | thiazol-4-yl | — [*] |
| A-270 | thiazol-5-yl | — [*] |
| A-271 | isothiazol-3-yl | — [*] |
| A-272 | isothiazol-4-yl | — [*] |
| A-273 | isothiazol-5-yl | — [*] |
| A-274 | 2-chlorothiazol-4-yl | — [*] |
| A-275 | 2-chlorothiazol-5-yl | — [*] |
| A-276 | 4-chlorothiazol-5-yl | — [*] |
| A-277 | 2-bromothiazol-4-yl | — [*] |
| A-278 | 2-bromothiazol-5-yl | — [*] |
| A-279 | 2,4-dichlorothiazol-5-yl | — [*] |
| A-280 | 3-chloroisothiazol-4-yl | — [*] |
| A-281 | 5-methylisothiazol-3-yl | — [*] |
| A-282 | 3-methylisothiazol-4-yl | — [*] |
| A-283 | 3-methylisothiazol-5-yl | — [*] |
| A-284 | 4,5-dichloroisothiazol-3-yl | — [*] |
| A-285 | 4,5-dimethylisothiazol-3-yl | — [*] |
| A-286 | 3,5-dimethylisothiazol-4-yl | — [*] |
| A-287 | 3,4-dichloroisothiazol-5-yl | — [*] |
| A-288 | oxazol-2-yl | — [*] |
| A-289 | oxazol-4-yl | — [*] |
| A-290 | oxazol-5-yl | — [*] |
| A-291 | isoxazol-3-yl | — [*] |
| A-292 | isoxazol-4-yl | — [*] |
| A-293 | isoxazol-5-yl | — [*] |
| A-294 | 3-chloroisoxazol-5-yl | — [*] |
| A-295 | 5-methylisoxazol-3-yl | — [*] |
| A-296 | 5-methylisoxazol-4-yl | — [*] |
| A-297 | 3-methylisoxazol-5-yl | — [*] |
| A-298 | 3,5-dimethylisoxazol-4-yl | — [*] |
| A-299 | 3-chloro-5-methylisoxazol-4-yl | — [*] |
| A-300 | 3-methyl-4-chloroisoxazol-5-yl | — [*] |
| A-301 | 1-methylpyrazol-3-yl | — [*] |
| A-302 | 1-methylpyrazol-4-yl | — [*] |
| A-303 | 1-methylpyrazol-5-yl | — [*] |
| A-304 | 1,3-dimethylpyrazol-4-yl | — [*] |
| A-305 | 1,5-dimethylpyrazol-4-yl | — [*] |
| A-306 | 1,3,5-trimethylpyrazol-4-yl | — [*] |
| A-307 | 1-methylimidazol-4-yl | — [*] |
| A-308 | 1,5-dimethylimidazol-4-yl | — [*] |
| A-309 | 1,2-dimethylimidazol-5-yl | — [*] |
| A-310 | 1,4-dimethylimidazol-5-yl | — [*] |
| A-311 | 1-indolyl | — [*] |
| A-312 | 2-fluoroindol-1-yl | — [*] |
| A-313 | 3-fluoroindol-1-yl | — [*] |
| A-314 | 4-fluoroindol-1-yl | — [*] |
| A-315 | 5-fluoroindol-1-yl | — [*] |
| A-316 | 6-fluoroindol-1-yl | — [*] |
| A-317 | 7-fluoroindol-1-yl | — [*] |
| A-318 | 2-chloroindol-1-yl | — [*] |
| A-319 | 3-chloroindol-1-yl | — [*] |
| A-320 | 4-chloroindol-1-yl | — [*] |
| A-321 | 5-chloroindol-1-yl | — [*] |
| A-322 | 6-chloroindol-1-yl | — [*] |
| A-323 | 7-chloroindol-1-yl | — [*] |
| A-324 | 2-methylindol-1-yl | — [*] |
| A-325 | 3-methylindol-1-yl | — [*] |
| A-326 | 4-methylindol-1-yl | — [*] |
| A-327 | 5-methylindol-1-yl | — [*] |
| A-328 | 6-methylindol-1-yl | — [*] |
| A-329 | 7-methylindol-1-yl | — [*] |
| A-330 | pyrazol-1-yl | — [*] |
| A-331 | 3-chloropyrazol-1-yl | — [*] |
| A-332 | 4-chloropyrazol-1-yl | — [*] |
| A-333 | 5-chloropyrazol-1-yl | — [*] |
| A-334 | 3-fluoropyrazol-1-yl | — [*] |
| A-335 | 4-fluoropyrazol-1-yl | — [*] |
| A-336 | 5-fluoropyrazol-1-yl | — [*] |
| A-337 | 3-methylpyrazol-1yl | — [*] |
| A-338 | 3-bromopyrazol-1-yl | — [*] |
| A-339 | 4-bromopyrazol-1-yl | — [*] |
| A-340 | 5-bromopyrazol-1yl | — [*] |
| A-341 | 2-fluoroimidazol-1-yl | — [*] |
| A-342 | 4-fluoroimidazol-1-yl | — [*] |
| A-343 | 5-fluoroimidazol-1-yl | — [*] |
| A-344 | 2-chloroimidazol-1-yl | — [*] |
| A-345 | 4-chloroimidazol-1-yl | — [*] |
| A-346 | 5-chloroimidazol-1-yl | — [*] |
| A-347 | 2-bromoimidazol-1-yl | — [*] |
| A-348 | 4-bromoimidazol-1-yl | — [*] |
| A-349 | 5-bromoimidazol-1-yl | — [*] |
| A-350 | pyrrol-1yl | — [*] |
| A-351 | 2-fluoropyrrol-1-yl | — [*] |
| A-352 | 3-fluoropyrrol-1-yl | — [*] |
| A-353 | 4-fluoropyrrol-1-yl | — [*] |
| A-354 | 5-fluoropyrrol-1-yl | — [*] |
| A-355 | 2-chloropyrrol-1-yl | — [*] |
| A-356 | 3-chloropyrrol-1-yl | — [*] |
| A-357 | 4-chloropyrrol-1-yl | — [*] |
| A-358 | 5-chloropyrrol-1-yl | — [*] |
| A-359 | 2-fluoropyrrol-1-yl | — [*] |
| A-360 | 3-bromopyrrol-1-yl | — [*] |
| A-361 | 4-bromopyrrol-1-yl | — [*] |
| A-362 | 5-bromopyrrol-1-yl | — [*] |
| A-363 | 2-methylpyrrol-1-yl | — [*] |
| A-364 | 3-methylpyrrol-1-yl | — [*] |
| A-365 | 4-methylpyrrol-1-yl | — [*] |
| A-366 | 5-methylpyrrol-1-yl | — [*] |

[*] “—” means that Y is a single bond

From the tables above, the compound names for the individual compounds are derived as follows: The “compound I.A.3aA-10” (emphasis added), for example, is the compound according to the invention of the formula I in which Z is CH₂(CH₂)₃CH₂, R², R³ and R⁴ are H and R⁵ is CH(CH₃)₂ (as stated in Table 3a) and R¹ is chloropyridin-3-yl and Y is O (as stated in row 10 of Table A).

The compounds of the formula I and the compositions according to the invention are suitable as fungicides for controlling harmful fungi. They are distinguished by excellent activity against a broad spectrum of phytopathogenic fungi including soilborne pathogens which originate in particular from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some of them are systemically active and can be used in crop protection as foliar fungicides, fungicides for seed dressing and soil fungicides. In addition, they are suitable for controlling fungi which, inter alia, attack the wood or the roots of plants.

The compounds I and the compositions according to the invention are of particular importance for the control of a large number of pathogenic fungi on various crop plants such as cereals, for example wheat, rye, barley, triticale, oats or rice; beets, for example sugar beets or fodder beets; pomaceous fruits, stone fruits and soft fruits, for example apples, pears, plums, peaches, almonds, cherries, strawberries, raspberries, currants or gooseberries; leguminous plants, for example beans, lentils, peas, lucerne or soybeans; oil plants, for example oilseed rape, mustard, olives, sunflowers, coconut, cocoa, castor beans, oil palms, peanuts or soybeans; cucurbits, for example pumpkins, cucumbers or melons; fiber plants, for example cotton, flax, hemp or jute; citrus fruits, for example oranges, lemons, grapefruits or mandarins; vegetable plants, for example spinach, lettuce, asparagus, cabbage plants, carrots, onions, tomatoes, potatoes, pumpkins or bell peppers; laurel plants, for example avocados, cinnamon or camphor; energy and raw material plants, for example corn, soybeans, wheat, oilseed rape, sugar cane or oil palms; corn; tobacco; nuts; coffee; tea; bananas; grapevines (grapes for eating and grapes for wine making); hops; grass, for example lawns; rubber plants; ornamental and forest plants, for example flowers, shrubs, deciduous trees and coniferous trees, and also on the propagation material, for example seeds, and on the harvested material of these plants.

Preferably, the compounds I and the compositions according to the invention are used for controlling a large number of fungal pathogens in agricultural crops, for example potatoes, sugar beets, tobacco, wheat, rye, barley, oats, rice, corn, cotton, soybeans, oilseed rape, leguminous plants, sunflowers, coffee or sugarcane; fruit plants, grapevines and ornamental plants and vegetables, for example cucumbers, tomatoes, beans and cucurbits and also on the propagation material, for example seeds, and the harvested products of these plants.

The term “plant propagation materials” includes all generative parts of the plant, for example seeds, and vegetative plant parts, such as seedlings and tubers (for example potatoes) which can be utilized for propagating a plant. These include seeds, roots, fruits, tubers, bulbs, rhizomes, shoots and other plant parts including seedlings and young plants which are transplanted after germination or after emergence. The young plants can be protected by partial or complete treatment, for example by immersion or watering, against harmful fungi.

The treatment of plant propagation materials with compounds I or the compositions according to the invention is used for controlling a large number of fungal pathogens in cereal crops, for example wheat, rye, barley or oats; rice, corn, cotton and soybeans.

The term crop plants also includes those plants which have been modified by breeding, mutagenesis or genetic engineering methods including the biotechnological agricultural products which are on the market or under development (see, for example, http://www.bio.orgispeeches/pubs/er/agri_products.asp). Genetically modified plants are plants whose genetic material has been modified in a manner which does not occur under natural conditions by crossing, mutations or by natural recombination (that is a recombination of the genetic information). In general, one or more genes are integrated into the genetic material of the plant in order to improve the properties of the plant. Such modifications by genetic engineering include post-translational modifications of proteins, oligopeptides or polypeptides, for example by glycosylation or attachment of polymers such as, for example, prenylated, acetylated or farnesylated radicals or PEG radicals.

By way of example, mention may be made of plants which, by breeding and genetic engineering, are tolerant to certain classes of herbicides, such as hydroxyphenylpyruvate dioxygenase (HPPD) inhibitors, acetolactate synthase (ALS) inhibitors, such as, for example, sulfonylureas (EP-A 257 993, U.S. Pat. No. 5,013,659) or imidazolinones (for example U.S. Pat. No. 6,222,100, WO 01/82685, WO 00/26390, WO 97/41218, WO 98/02526, WO 98/02527, WO 04/106529, WO 05/20673,

WO 03/14357, WO 03/13225, WO 03/14356, WO 04/16073), enolpyruvylshikimate 3-phosphate synthase (EPSPS) inhibitors, such as, for example, glyphosate (see, for example, WO 92/00377), glutamine synthetase (GS) inhibitors, such as, for example, glufosinate (see, for example, EP-A 242 236, EP-A 242 246) or oxynil herbicides (see, for example, U.S. Pat. No. 5,559,024). Clearfield° oilseed rape (BASF SE, Germany), for example, which is tolerant to imidazolinones, for example imazamox, was generated by breeding and mutagenesis. With the aid of genetic engineering methods, crop plants such as soybeans, cotton, corn, beets and oilseed rape were generated which are resistant to glyphosate or glufosinate, and which are obtainable under the trade names RoundupReady® (glyphosate-resistant, Monsanto, U.S.A.) and Liberty Link° (glufosinate-resistant, Bayer CropScience, Germany).

Also included are plants which, owing to interventions by genetic engineering, produce one or more toxins, for example those of the bacterial strain Bacillus. Toxins which are produced by such genetically modified plants include, for example, insecticidal proteins of Bacillus spp., in particular B. thuringiensis, such as the endotoxins Cry1Ab, Cry1Ac, Cry1F, Cry1Fa2, Cry2Ab, Cry3A, Cry3Bb1, Cry9c, Cry34Ab1 or Cry35Ab1; or vegetative insecticidal proteins (VIPs), for example VIP1, VIP2, VIP3, or VIP3A; insecticidal proteins of nematode-colonizing bacteria, for example Photorhabdus spp. or Xenorhabdus spp.; toxins of animal organisms, for example wasp, spider or scorpion toxins; fungal toxins, for example from Streptomycetes; plant lectins, for example from peas or barley; agglutinins; protease inhibitors, for example trypsin inhibitors, serine protease inhibitors, patatin, cystatin or papain inhibitors; ribosome-inactivating proteins (RIPs), for example ricin, corn-RIP, abrin, luffin, saporin or bryodin; steroid-metabolizing enzymes, for example 3-hydroxysteroid oxidase, ecdysteroid-IDP glycosyl transferase, cholesterol oxidase, ecdyson inhibitors, or HMG-CoA reductase; ion channel blockers, for example inhibitors of sodium channels or calcium channels; juvenile hormone esterase; receptors of the diuretic hormone (helicokinin receptors); stilbene synthase, bibenzyl synthase, chitinases and glucanases. In the plants, these toxins may also be produced as pretoxins, hybrid proteins or truncated or otherwise modified proteins. Hybrid proteins are characterized by a novel combination of different protein domains (see, for example, WO 2002/015701). Further examples of such toxins or genetically modified plants which produce these toxins are disclosed in EP-A 374 753, WO 93/07278, WO 95/34656, EP-A 427 529, EP-A 451 878, WO 03/18810 and WO 03/52073. The methods for producing these genetically modified plants are known to the person skilled in the art and disclosed, for example, in the publications mentioned above. Many of the toxins mentioned above bestow, upon the plants by which they are produced, tolerance to pests from all taxonomic classes of arthropods, in particular to beetles (Coeleropta), dipterans (Diptera) and butterflies (Lepidoptera) and to nematodes (Nematoda). Genetically modified plants which produce one or more genes coding for insecticidal toxins are described, for example, in the publications mentioned above, and some of them are commercially available, such as, for example, YieldGard® (corn varieties producing the toxin Cry1Ab), YieldGard® Plus (corn varieties which produce the toxins CrylAb and Cry3Bb1), Starlink® (corn varieties which produce the toxin Cry9c), Herculex® RW (corn varieties which produce the toxins Cry34Ab1, Cry35Ab1 and the enzyme phosphinothricin-N-acetyltransferase [PAT]); NuCOTN® 33B (cotton varieties which produce the toxin CrylAc), Bollgard® I (cotton varieties which produce the toxin CrylAc), Bollgard® II (cotton varieties which produce the toxins CrylAc and Cry2Ab2); VIPCOT® (cotton varieties which produce a VIP toxin); NewLeaf® (potato varieties which produce the toxin Cry3A); Bt-Xtra®, NatureGard®, KnockOut®, BiteGard®, Protecta®, Bt11 (for example Agrisure® CB) and Bt176 from Syngenta Seeds SAS, France (corn varieties which produce the toxin CrylAb and the PAT enzyme), MIR604 from Syngenta Seeds SAS, France (corn varieties which produce a modified version of the toxin Cry3A, see WO 03/018810), MON 863 from Monsanto Europe S.A., Belgium (corn varieties which produce the toxin Cry3Bb1), IPC 531 from Monsanto Europe S.A., Belgium (cotton varieties which produce a modified version of the toxin Cry1Ac) and 1507 from Pioneer Overseas Corporation, Belgium (corn varieties which produce the toxin Cry1F and the PAT enzyme).

Also included are plants which, with the aid of genetic engineering, produce one or more proteins which have increased resistance to bacterial, viral or fungal pathogens, such as, for example, pathogenesis-related proteins (PR proteins, see EP-A 0 392 225), resistance proteins (for example potato varieties producing two resistance genes against Phytophthora infestans from the wild Mexican potato Solanum bulbocastanum) or T4 lysozyme (for example potato varieties which, by producing this protein, are resistant to bacteria such as Erwinia amylvora).

Also included are plants whose productivity has been improved with the aid of genetic engineering methods, for example by enhancing the potential yield (for example biomass, grain yield, starch, oil or protein content), tolerance to drought, salt or other limiting environmental factors or resistance to pests and fungal, bacterial and viral pathogens.

Also included are plants whose ingredients have been modified with the aid of genetic engineering methods in particular for improving human or animal diet, for example by oil plants producing health-promoting long-chain omega 3 fatty acids or monounsaturated omega 9 fatty acids (for example Nexera® oilseed rape, DOW Agro Sciences, Canada).

Also included are plants which have been modified with the aid of genetic engineering methods for improving the production of raw materials, for example by increasing the amylopectin content of potatoes (Amflora® potato, BASF SE, Germany).

Specifically, the compounds I and, respectively, the compositions according to the invention are suitable for controlling the following plant diseases:

Albugo spp. (white rust) on ornamental plants, vegetable crops (for example A. candida) and sunflowers (for example A. tragopogonis);

Alternaria spp. (black spot disease, black blotch) on vegetables, oilseed rape (for example A. brassicola or A. brassicae), sugar beet (for example A. tenuis), fruit, rice, soybeans and also on potatoes (for example A. solani or A. alternate) and tomatoes (for example A. solani or A. alternate) and Alternaria spp. (black head) on wheat;

Aphanomyces spp. on sugar beet and vegetables;

Ascochyta spp. on cereals and vegetables, for example A. tritici (Ascochyta leaf blight) on wheat and A. hordei on barley;

Bipolaris and Drechslera spp. (teleomorph: Cochliobolus spp.) for example leaf spot diseases (D. maydis and B. zeicola) on corn, for example glume blotch (B. sorokiniana) on cereals and for example B. oryzae on rice and on lawn;

Blumeria (old name: Erysiphe) graminis (powdery mildew) on cereals (for example wheat or barley);

Botryosphaeria spp. (‘Black Dead Arm Disease’) on grapevines (for example B. obtusa);

Botrytis cinerea (teleomorph: Botryotinia fuckeliana: gray mold, gray rot) on soft fruit and pome fruit (inter alia strawberries), vegetables (inter alia lettuce, carrots, celeriac and cabbage), oilseed rape, flowers, grapevines, forest crops and wheat (ear mold);

Bremia lactucae (downy mildew) on lettuce;

Ceratocystis (syn. Ophiostoma) spp. (blue stain fungus) on deciduous trees and coniferous trees, for example C. ulmi (Dutch elm disease) on elms;

Cercospora spp. (Cercospora leaf spot) on corn (for example C. zeae-maydis), rice, sugar beet (for example C. beticola), sugar cane, vegetables, coffee, soybeans (for example C. sojina or C. kikuchii) and rice;

Cladosporium spp. on tomato (for example C. fulvum: tomato leaf mold) and cereals, for example C. herbarum (ear rot) on wheat;

Claviceps purpurea (ergot) on cereals;

Cochliobolus (anamorph: Helminthosporium or Bipolaris) spp. (leaf spot) on corn (for example C. carbonum), cereals (for example C. sativus, anamorph: B. sorokiniana: glume blotch) and rice (for example C. miyabeanus, anamorph: H. oryzae);

Colletotrichum (teleomorph: Glomerella) spp. (anthracnosis) on cotton (for example C. gossypii), corn (for example C. graminicola: stem rot and anthracnosis), soft fruit, potatoes (for example C. coccodes: wilt disease), beans (for example C. lindemuthianum) and soybeans (for example C. truncatum);

Corticium spp., for example C. sasakii (sheath blight) on rice;

Corynespora cassiicola (leaf spot) on soybeans and ornamental plants;

Cycloconium spp., for example C. oleaginum on olive; Cylindrocarpon spp. (for example fruit tree cancer or black foot disease of grapevine, teleomorph: Nectria or Neonectria spp.) on fruit trees, grapevines (for example C. liriodendri, teleomorph: Neonectria liriodendri, black foot disease) and many ornamental trees;

Dematophora (teleomorph: Rosellinia) necatrix (root/stem rot) on soybeans;

Diaporthe spp. for example D. phaseolorum (stem disease) on soybeans;

Drechslera (syn. Helminthosporium, teleomorph: Pyrenophora) spp. on corn, cereals, such as barley (for example D. teres, net blotch) and on wheat (for example D. tritici-repentis: DTR leaf spot), rice and lawn;

Esca disease (dieback of grapevine, apoplexia) on grapevines, caused by Formitiporia (syn. Phellinus) punctata, F. mediterranea, Phaeomoniella chlamydospora (old name Phaeoacremonium chlamydosporum), Phaeoacremonium aleophilum and/or Botryosphaeria obtusa; Elsinoe spp. on pome fruit (E. pyri) and soft fruit (E. veneta: anthracnosis) and also grapevines (E. ampelina: anthracnosis);

Entyloma oryzae (leaf smut) on rice;

Epicoccum spp. (black head) on wheat; Eysiphe spp. (powdery mildew) on sugar beet (E. betae), vegetables (for example E. pisi), such as cucumber species (for example E. cichoracearum) and cabbage species, such as oilseed rape (for example E. cruciferarum);

Eutypa lata (Eutypa cancer or dieback, anamorph: Cytosporina lata, syn. Libertella blepharis) on fruit trees, grapevines and many ornamental trees;

Exserohilum (syn. Helminthosporium) spp. on corn (for example E. turcicum);

Fusarium (teleomorph: Gibberella) spp. (wilt disease, root and stem rot) on various plants, such as for example F. graminearum or F. culmorum (root rot and silver-top) on cereals (for example wheat or barley), F. oxysporum on tomatoes, F. solani on soybeans and F. verticillioides on corn;

Gaeumannomyces graminis (take-all) on cereals (for example wheat or barley) and corn; Gibberella spp. on cereals (for example G. zeae) and rice (for example G. fujikuroi: bakanae disease);

Glomerella cingulata on grapevines, pome fruit and other plants and G. gossypii on cotton; Grainstaining complex on rice;

Guignardia bidwellii (black rot) on grapevines; Gymnosporangium spp. on Rosaceae and juniper, for example G. sabinae (pear rust) on pears;

Helminthosporium spp. (syn. Drechslera, teleomorph: Cochliobolus) on corn, cereals and rice; Hemileia spp., for example H. vastatrix (coffee leaf rust) on coffee;

Isariopsis clavispora (syn. Cladosporium vitis) on grapevines;

Macrophomina phaseolina (syn. phaseoli) (root/stem rot) on soybeans and cotton; Microdochium (syn. Fusarium nivale (pink snow mold) on cereals (for example wheat or barley);

Microsphaera diffusa (powdery mildew) on soybeans;

Monilinia spp., for example M. laxa, M. fructicola and M. fructigena (blossom and twig blight) on stone fruit and other Rosaceae; Mycosphaerella spp. on cereals, bananas, soft fruit and peanuts, such as for example M. graminicola (anamorph: Septoria tritici, Septoria leaf blotch) on wheat or M. fijiensis (sigatoka disease) on bananas;

Peronospora spp. (downy mildew) on cabbage (for example P. brassicae), oilseed rape (for example P. parasitica), bulbous plants (for example P. destructor), tobacco (P. tabacina) and soybeans (for example P. manshurica);

Phakopsora pachyrhizi and P. methomiae (soybean rust) on soybeans;

Phialophora spp. for example on grapevines (for example P. tracheiphila and P. tetraspora) and soybeans (for example P. gregata: stem disease);

Phoma lingam (root and stem rot) on oilseed rape and cabbage and P. betae (leaf spot) on sugar beet;

Phomopsis spp. on sunflowers, grapevines (for example P. viticola: dead-arm disease) and soybeans (for example stem canker/stem blight: P. phaseoli, teleomorph: Diaporthe phaseolorum);

Physoderma maydis (brown spot) on corn;

Phytophthora spp. (wilt disease, root, leaf, stem and fruit rot) on various plants, such as on bell peppers and cucumber species (for example P. capsici), soybeans (for example P. megasperma, syn. P. sojae), potatoes and tomatoes (for example P. infestans: late blight and brown rot) and deciduous trees (for example P. ramorum: sudden oak death);

Plasmodiophora brassicae (club-root) on cabbage, oilseed rape, radish and other plants;

Plasmopara spp., for example P. viticola (peronospora of grapevines, downy mildew) on grapevines and P. halstedii on sunflowers; Podosphaera spp. (powdery mildew) on Rosaceae, hops, pome fruit and soft fruit, for example P. leucotricha on apple;

Polymyxa spp., for example on cereals, such as barley and wheat (P. graminis) and sugar beet (P. betae) and the viral diseases transmitted thereby;

Pseudocercosporella herpotrichoides (eyespot/stem break, teleomorph: Tapesia yallundae) on cereals, for example wheat or barley;

Pseudoperonospora (downy mildew) on various plants, for example P. cubensis on cucumber species or P. humili on hops;

Pseudopezicula tracheiphila (angular leaf scorch, anamorph: Phialophora) on grapevines;

Puccinia spp. (rust disease) on various plants, for example P. triticina (brown rust of wheat), P. striiformis (yellow rust), P. hordei (dwarf leaf rust), P. graminis (black rust) or P. recondita (brown rust of rye) on cereals, such as for example wheat, barley or rye, and on asparagus (for example P. asparagi);

Pyrenophora (anamorph: Drechslera) tritici-repenttis (speckled leaf blotch) on wheat or P. teres (net blotch) on barley;

Pyricularia spp., for example P. oryzae (teleomorph: Magnaporthe grisea, rice blast) on rice and P. grisea on lawn and cereals;

Pythium spp. (damping-off disease) on lawn, rice, corn, wheat, cotton, oilseed rape, sunflowers, sugar beet, vegetables and other plants (for example P. ultimum or P. aphanidermatum);

Ramularia spp., for example R. collo-cygni (Ramularia leaf and lawn spot/physiological leaf spot) on barley and R. beticola on sugar beet;

Rhizoctonia spp. on cotton, rice, potatoes, lawn, corn, oilseed rape, potatoes, sugar beet, vegetables and on various other plants, for example R. solani (root and stem rot) on soybeans, R. solani (sheath blight) on rice or R. cerealis (sharp eyespot) on wheat or barley;

Rhizopus stolonifer (soft rot) on strawberries, carrots, cabbage, grapevines and tomato; Rhynchosporium secalis (leaf spot) on barley, rye and triticale;

Sarocladium oryzae and S. attenuatum (sheath rot) on rice;

Sclerotinia spp. (stem or white rot) on vegetable and field crops, such as oilseed rape, sunflowers (for example Sclerotinia sclerotiorum) and soybeans (for example S. rolfsii,

Septoria spp. on various plants, for example S. glycines (leaf spot) on soybeans, S. tritici(Septoria leaf blotch) on wheat and S. (syn. Stagonospora) nodorum (leaf blotch and glume blotch) on cereals;

Uncinula (syn. Erysiphe) necator(powdery mildew, anamorph: Oidium tuckeri) on grapevines;

Setosphaeria spp. (leaf spot) on corn (for example S. turcicum, syn. Helminthosporium turcicum) and lawn;

Sphacelotheca spp. (head smut) on corn, (for example S. reiliana: kernel smut), millet and sugar cane;

Sphaerotheca fuliginea (powdery mildew) on cucumber species;

Spongospora subterranea (powdery scab) on potatoes and the viral diseases transmitted thereby;

Stagonospora spp. on cereals, for example S. nodorum (leaf blotch and glume blotch, teleomorph: Leptosphaeria [syn. Phaeosphaeria] nodorum) on wheat;

Synchytrium endobioticum on potatoes (potato wart disease);

Taphrina spp., for example T. deformans (curly-leaf disease) on peach and T. pruni (plum-pocket disease) on plums;

Thielaviopsis spp. (black root rot) on tobacco, pome fruit, vegetable crops, soybeans and cotton, for example T. basicola (syn. Chalara elegans); Tilletia spp. (bunt or stinking smut) on cereals, such as for example T. tritici (syn. T. caries, wheat bunt) and T. controversa (dwarf bunt) on wheat;

Typhula incarnata (gray snow mold) on barley or wheat;

Urocystis spp., for example U occulta (flag smut) on rye;

Uromyces spp. (rust) on vegetable plants, such as beans (for example U. appendiculatus, syn. U. phaseoli) and sugar beet (for example U. betae);

Ustilago spp. (loose smut) on cereals (for example U. nuda and U. avaenae), corn (for example U. maydis: corn smut) and sugar cane;

Venturia spp. (scab) on apples (for example V. inaequalis) and pears and

Verticillium spp. (leaf and shoot wilt) on various plants, such as fruit trees and ornamental trees, grapevines, soft fruit, vegetable and field crops, such as for example V. dahliae on strawberries, oilseed rape, potatoes and tomatoes.

Moreover, the compounds I and the compositions according to the invention are suitable for controlling harmful fungi in the protection of stored products (also of harvested products) and in the protection of materials and buildings. The term “protection of materials and buildings” comprises the protection of technical and nonliving materials, such as, for example, adhesives, sizes, wood, paper and cardboard, textiles, leather, paint dispersions, plastic, cooling lubricants, fibers and tissues, against attack and destruction by unwanted microorganisms such as fungi and bacteria. In the protection of wood and materials, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of materials to the following yeast fungi: Candida spp. and Saccharomyces cerevisae.

The compounds of the formula I may be present in various crystal modifications which may differ in their biological activity. These are included in the scope of the present invention.

The compounds I and the compositions according to the invention are suitable for improving plant health. Moreover, the invention relates to a method for improving plant health by treating the plants, the plant propagation material and/or the site at which the plants grow or are intended to grow with an effective amount of the compounds I or the compositions according to the invention.

The term “plant health” comprises states of a plant and/or its harvested material which are determined by various indicators individually or in combination, such as, for example, yield (for example increased biomass and/or increased content of utilizable ingredients), plant vitality (for example increased plant growth and/or greener leaves (“greening effect”)), quality (for example increased content or composition of certain ingredients) and tolerance to biotic and/or abiotic stress. The indicators mentioned here for a state of plant health may occur independently of one another or may influence each other.

The compounds I are employed as such or in the form of a composition by treating the harmful fungi, their habitat or the plants or plant propagation materials, for example seed materials to be protected against fungal attack, the soil, areas, materials or spaces with a fungicidally effective amount of the compounds I. The application can be carried out both before and after the infection of the plants, plant propagation materials, for example seed materials, the soil, the areas, materials or spaces by the fungi.

Plant propagation materials can be treated prophylactically during or even before sowing or during or even before transplanting with compounds I as such or with a composition comprising at least one compound I.

The invention furthermore relates to agrochemical compositions comprising a solvent or solid carrier and at least one compound I, and also to their use for controlling harmful fungi.

An agrochemical composition comprises a fungicidally effective amount of a compound I. The term “effective amount” refers to an amount of the agrochemical composition or of the compound I which is sufficient for controlling harmful fungi on crop plants or in the protection of materials and buildings and does not cause any significant damage to the treated crop plants. Such an amount may vary within a wide range and is influenced by numerous factors, such as, for example, the harmful fungus to be controlled, the respective crop plant or materials treated, the climatic conditions and compounds.

The compounds I, their N-oxides and their salts can be converted into the types customary for agrochemical compositions, for example solutions, emulsions, suspensions, dusts, powders, pastes and granules. The type of composition depends on the respective intended purpose; in each case, it should ensure a fine and even distribution of the compound according to the invention.

Here, examples of types of compositions are suspensions (SC, OD, FS), emulsifiable concentrates (EC), emulsions (EW, EO, ES), pastes, pastilles, wettable powders or dusts (WP, SP, SS, WS, DP, DS) or granules (GR, FG, GG, MG) which may either be water-soluble or dispersible (wettable), and also gels for treating plant propagation materials such as seed (GF).

In general, the composition types (for example EC, SC, OD, FS, WG, SG, WP, SP, SS, WS, GF) are used in diluted form. Composition types such as DP, DS, GR, FG, GG and MG are generally employed in undiluted form.

The agrochemical compositions are prepared in a known manner (see, for example, U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th edition, McGraw-Hill, New York, 1963, 8-57 and ff., WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (John Wiley & Sons, New York, 1961), Hance et al.: Weed Control Handbook (8th Ed., Blackwell Scientific Publications, Oxford, 1989) and Mollet, H. and Grubemann, A.: Formulation technology (Wiley VCH Verlag, Weinheim, 2001).

The agrochemical compositions may furthermore also comprise auxiliaries customary for crop protection compositions, the selection of the auxiliaries depending on the use form or the active compound in question.

Examples of suitable auxiliaries are solvents, solid carriers, surfactants (such as further solubilizers, protective colloids, wetting agents and tackifiers), organic and inorganic thickeners, bactericides, antifreeze agents, antifoams, optionally colorants and adhesives (for example for the treatment of seed).

Suitable solvents are water, organic solvents, such as mineral oil fractions having a medium to high boiling point, such as kerosene and diesel oil, furthermore coal tar oils, and also oils of vegetable or animal origin, aliphatic, cyclic and aromatic hydrocarbons, for example paraffins, tetrahydronaphthalene, alkylated naphthalenes and derivatives thereof, alkylated benzenes and derivatives thereof, alcohols, such as methanol, ethanol, propanol, butanol and cyclohexanol, glycols, ketones, such as cyclohexanone, gamma-butyrolactone, dimethyl fatty amides, fatty acids and fatty acid esters and strongly polar solvents, for example amines, such as N-methylpyrrolidone. In principle, it is also possible to use solvent mixtures, and also mixtures of the solvents mentioned above and water.

Solid carriers are mineral earths, such as silicic acids, silica gels, silicates, talc, kaolin, limestone, lime, chalk, bole, loess, clay, dolomite, diatomaceous earth, calcium sulfate and magnesium sulfate, magnesium oxide, ground synthetic substances, fertilizers, such as ammonium sulfate, ammonium phosphate, ammonium nitrate, ureas and vegetable products, such as cereal meal, tree bark meal, sawdust and nutshell meal, cellulose powder or other solid carriers.

Suitable surfactants (adjuvants, wetting agents, tackifiers, dispersants or emulsifiers) are the alkali metal, alkaline earth metal and ammonium salts of aromatic sulfonic acids, for example of lignosulfonic acid (Borresperse® types, Borregaard, Norway), phenolsulfonic acid, naphthalenesulfonic acid (Morwet® types, Akzo Nobel, USA) and dibutylnaphthalenesulfonic acid (Nekal® types, BASF, Germany), and also of fatty acids, alkyl- and alkylarylsulfonates, alkyl, lauryl ether and fatty alcohol sulfates, and also salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octyl phenol ether, ethoxylated isooctylphenol, octylphenol or nonylphenol, alkylphenyl polyglycol ether, tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors, and also proteins, denatured proteins, polysaccharides (for example methylcellulose), hydrophobically modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethyleneimine (Lupasol® types, BASF, Germany), polyvinylpyrrolidone and copolymers thereof.

Examples of thickeners (i.e. compounds which impart modified flow properties to the composition, i.e. high viscosity in the state of rest and low viscosity in motion) are polysaccharides and also organic and inorganic sheet minerals, such as xanthan gum (Kelzan®, CP Kelco, USA), Rhodopol® 23 (Rhodia, France) or Veegum® (R.T. Vanderbilt, USA) or Attaclay® (Engelhard Corp., NJ, USA).

Bactericides can be added for stabilizing the composition. Examples of bactericides are bactericides based on dichlorophen and benzyl alcohol hemiformal (Proxel® from ICI or Acticide® RS from Thor Chemie and Kathon® MK from Rohm & Haas), and also isothiazolinone derivatives, such as alkylisothiazolinones and benzisothiazolinones (Acticide® MBS from Thor Chemie).

Examples of suitable antifreeze agents are ethylene glycol, propylene glycol, urea and glycerol.

Examples of antifoams are silicone emulsions (such as, for example, Silikon SRE, Wacker, Germany or Rhodorsil®, Rhodia, France), long-chain alcohols, fatty acids, salts of fatty acids, organofluorine compounds and mixtures thereof.

Examples of colorants are both sparingly water-soluble pigments and water-soluble dyes. Examples which may be mentioned are the dyes and pigments known under the names Rhodamin B, C. I. Pigment Red 112 and C. I. Solvent Red 1, Pigment blue 15:4, Pigment blue 15:3, Pigment blue 15:2, Pigment blue 15:1, Pigment blue 80, Pigment yellow 1, Pigment yellow 13, Pigment red 48:2, Pigment red 48:1, Pigment red 57:1, Pigment red 53:1, Pigment orange 43, Pigment orange 34, Pigment orange 5, Pigment green 36, Pigment green 7, Pigment white 6, Pigment brown 25, Basic violet 10, Basic violet 49, Acid red 51, Acid red 52, Acid red 14, Acid blue 9, Acid yellow 23, Basic red 10, Basic red 108.

Examples of adhesives are polyvinylpyrrolidone, polyvinyl acetate, polyvinyl alcohol and cellulose ether (Tylose®, Shin-Etsu, Japan).

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydro-naphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the compounds I and, if present, further active compounds with at least one solid carrier.

Granules, for example coated granules, impregnated granules and homogeneous granules, can be prepared by binding the active compounds to at least one solid carrier. Solid carriers are, for example, mineral earths, such as silica gels, silicates, talc, kaolin, attaclay, limestone, lime, chalk, bole, loess, clay, dolomite, diatomaceous earth, calcium sulfate and magnesium sulfate, magnesium oxide, ground synthetic substances, fertilizers, such as ammonium sulfate, ammonium phosphate, ammonium nitrate, ureas and vegetable products, such as cereal meal, tree bark meal, sawdust and nutshell meal, cellulose powder or other solid carriers.

The following are examples of types of composition:

1. Types of composition for dilution with water

i) Water-soluble concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a composition having an active compound content of 10% by weight.

ii) Dispersible concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight.

iii) Emulsifiable concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The composition has an active compound content of 15% by weight.

iv) Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The composition has an active compound content of 25% by weight.

v) Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the composition is 20% by weight.

vi) Water-dispersible granules and water-soluble granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The composition has an active compound content of 50% by weight.

vii) Water-dispersible powders and water-soluble powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the composition is 75% by weight.

viii) Gels (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

2. Types of composition to be applied undiluted

ix) Dusts (DP, DS)

5 parts by weight of the active compounds are ground finel_{y} and mixed intimately with 95 parts by weight of finely divided kaolin. This gives a dustable product with an active compound content of 5% by weight.

x) Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

xi) ULV solutions (UL)

10 parts by weight of the active compounds are dissolved in 90 parts by weight of an organic solvent, for example xylene. This gives a composition with an active compound content of 10% by weight to be applied undiluted.

In general, the compositions of the compounds according to the invention comprise from 0.01 to 95% by weight, preferably from 0.1 to 90% by weight, of the compounds I. The compounds are preferably employed in a purity of from 90% to 100%, preferably 95% to 100%.

Water-soluble concentrates (LS), suspensions (FS), dusts (DS), water-dispersible and water-soluble powders (WS, SS), emulsions (ES), emulsifiable concentrates (EC) and gels (GF) are usually used for the treatment of plant propagation materials, in particular seed. These compositions can be applied to the propagation materials, in particular seed, in undiluted or, preferably, diluted form. In this case, the corresponding composition can be diluted 2 to 10 times so that in the compositions used for the seed dressing from 0.01 to 60% by weight, preferably from 0.1 to 40% by weight of active compound are present. The application can be carried out before or during sowing. The treatment of plant propagation material, in particular the treatment of seed, is known to the person skilled in the art and is carried out by dusting, coating, pelleting, dipping or drenching the plant propagation material, the treatment preferably being carried out by pelleting, coating and dusting or by furrow treatment, such that, for example, premature germination of the seed is prevented.

For seed treatment, preference is given to using suspensions. Such compositions usually comprise from 1 to 800 g of active compound/I, from 1 to 200 g of surfactants/I, from 0 to 200 g of antifreeze agent/I, from 0 to 400 g of binders/I, from 0 to 200 g of colorants/I and solvents, preferably water.

The compounds can be used as such or in the form of their compositions, for example in the form of directly sprayable solutions, powders, suspensions, dispersions, emulsions, oil dispersions, pastes, dustable products, materials for spreading or granules, by means of spraying, atomizing, dusting, spreading, raking in, immersing or pouring. The types of composition depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply compositions comprising over 95% by weight of active compound, or even to apply the active compound without additives.

When used in crop protection, the application rates are from 0.001 to 2.0 kg of active compound per ha, preferably from 0.005 to 2 kg per ha, particularly preferably from 0.05 to 0.9 kg per ha, especially from 0.1 to 0.75 kg per ha, depending on the nature of the desired effect.

In the treatment of plant propagation materials, for example seed, the amounts of active compound used are generally from 0.1 to 1000 g/100 kg of propagation material or seed, preferably from 1 to 1000 g/100 kg, particularly preferably from 1 to 100 g/100 kg, especially from 5 to 100 g/100 kg.

When used in the protection of materials or stored products, the active compound application rate depends on the kind of application area and on the desired effect. Amounts typically applied in the protection of materials are, for example, from 0.001 g to 2 kg, preferably from 0.005 g to 1 kg, of active compound per cubic meter of treated material.

Various types of oils, wetters, adjuvants, herbicides, bactericides, other fungicides and/or pesticides may be added to the active compounds or the compositions comprising them, optionally not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention in the application form as fungicides can also be present together with other active compounds, for example with herbicides, insecticides, growth regulators, fungicides or else with fertilizers, as premix or optionally also only immediately prior to use (tank mix).

When mixing the compounds I or the compositions comprising them with one or more further active compounds, in particular fungicides, it is in many cases possible, for example, to widen the activity spectrum or to prevent the development of resistance. In many cases, synergistic effects are obtained.

The following list of active compounds with which the compounds according to the invention can be applied together is meant to illustrate the possible combinations, but not to limit them:
A) strobilurins:
   azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methylphenoxy)-5-fluoropyrimidin-4-yloxy)phenyl)-2-methoxyimino-N-methylacetamide, methyl 2-(ortho-((2,5-dimethylphenyloxy-methylene)phenyl)-3-methoxyacrylate, methyl 3-methoxy-2-(2-(N-(4-methoxyphenyl)-cyclopropanecarboximidoylsulfanylmethyl)phenyl)acrylate, 2-(2-(3-(2,6-dichloro-phenyl)-1-methylallylideneaminooxymethyl)phenyl)-2-methoxyimino-N-methyl-acetamide;
B) carboxamides:
   carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penflufen (N-(2-(1,3-dimethylbutyl)phenyl)-1,3-dimethyl-5-fluoro-1H-pyrazole-4-carboxamide, penthiopyrad, sedaxane, tecloftalam, thifluzamide, tiadinil, 2-amino-4-methylthiazole-5-carboxanilide, 2-chloro-N-(1,1,3-trimethylindan-4-yl)nicotinamide, N-(3′,4′,5′-trifluorobiphenyl-2-yl)-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(2-(1,3,3-trimethylbutyl)phenyl)-1,3-dimethyl-5-fluoro-1H-pyrazole-4-carboxamide;
   carboxylic acid morpholides: dimethomorph, flumorph, pyrimorph;
   benzamides: flumetover, fluopicolide, fluopyram, zoxamide, N-(3-ethyl-3,5,5-tri-methylcyclohexyl)-3-formylamino-2-hydroxybenzamide;
   other carboxamides: carpropamid, diclocymet, mandipropamid, oxytetracyclin, silthiofam, N-(6-methoxypyridin-3-yl)cyclopropanecarboxamide;
C) azoles:
   triazoles: azaconazole, bitertanole, bromuconazole, cyproconazole, difenoconazole, diniconazole, diniconazole-M, epoxiconazole, fenbuconazole, fluquinconazole, flusilazole, flutriafole, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenole, triticonazole, uniconazole, 1-(4-chlorophenyl)-2-([1,2,4]triazol-1-yl)cycloheptanol;
   imidazoles: cyazofamid, imazalil, imazalil sulfate, pefurazoate, prochloraz, triflumizole;
   benzimidazoles: benomyl, carbendazim, fuberidazole, thiabendazole;
   others: ethaboxam, etridiazole, hymexazole, 2-(4-chlorophenyl)-N-[4-(3,4-dimethoxyphenyl)isoxazol-5-yl]-2-prop-2-inyloxyacetamide;
D) nitrogenous heterocyclyl compounds
   pyridines: fluazinam, pyrifenox, 3-[5-(4-chlorophenyl)-2,3-dimethylisoxazolidin-3-yl]-pyridine, 3-[5-(4-methylphenyl)-2,3-dimethylisoxazolidin-3-yl]pyridine, 2,3,5,6-tetra-chloro-4-methanesulfonylpyridine, 3,4,5-trichloropyridine-2,6-dicarbonitrile, N-(1-(5-bromo-3-chloropyridin-2-yl)ethyl)-2,4-dichloronicotinamide, N-((5-bromo-3-chloropyridin-2-yl)methyl)-2,4-dichloronicotinamide;
   pyrimidines: bupirimate, cyprodinil, diflumetorim, fenarimol, ferimzone, mepanipyrim, nitrapyrin, nuarimol, pyrimethanil;
   piperazines: triforine;
   pyrroles: fludioxonil, fenpiclonil;
   morpholines: aldimorph, dodemorph, dodemorph acetate, fenpropimorph, tridemorph;
   piperidines: fenpropidin;
   dicarboximides: fluoroimide, iprodione, procymidone, vinclozolin;
   nonaromatic 5-membered heterocycles: famoxadone, fenamidone, flutianil, octhilinone, probenazole, S-allyl 5-amino-2-isopropyl-3-oxo-4-ortho-tolyl-2,3-dihydropyrazole-1-thiocarboxylate;
   others: acibenzolar-5-methyl, amisulbrom, anilazine, blasticidin-S, captafol, captan, quinomethionate, dazomet, debacarb, diclomezine, difenzoquat, difenzoquat methylsulfate, fenoxanil, folpet, oxolinic acid, piperalin, proquinazid, pyroquilone, quinoxyfen, triazoxide, tricyclazole, 2-butoxy-6-iodo-3-propylchromen-4-one, 5-chloro-1-(4,6-dimethoxypyrimidin-2-yl)-2-methyl-1H-benzimidazole, 5-chloro-7-(4-methylpiperidin-1-yl)-6-(2,4,6-trifluorophenyl)-[1,2,4]triazolo[1,5-a]pyrimidine, 5-ethyl-6-octyl-[1,2,4]triazolo[1,5-a]pyrimidin-7-ylamine;
E) carbamates and dithiocarbamates
   thio- and dithiocarbamates: ferbam, mancozeb, maneb, metam, methasulfocarb, metiram, propineb, thiram, zineb, ziram;
   carbamates: diethofencarb, benthiavalicarb, iprovalicarb, propamocarb, propamocarb hydrochloride, valiphenal, 4-fluorophenyl N-(1-(1-(4-cyanophenyl)ethanesulfonyl)but-2-yl)carbamate;
F) other fungicides
   guanidines: dodine, dodine free base, guazatine, guazatine acetate, iminoctadine, iminoctadine triacetate, iminoctadine tris(albesilate);
   antibiotics: kasugamycin, kasugamycin hydrochloride hydrate, polyoxins, streptomycin, validamycin A;
   nitrophenyl derivatives:
      binapacryl, dicloran, dinobuton, dinocap, nitrothal isopropyl, tecnazene;
   organometallic compounds: fentin salts, such as, for example, fentin acetate, fentin chloride, fentin hydroxide;
   sulfur-comprising heterocyclyl compounds: dithianon, isoprothiolane;
   organophosphorus compounds: edifenphos, fosetyl, fosetyl aluminum, iprobenfos, phosphorous acid and its salts, pyrazophos, tolclofos-methyl;
   organochlorine compounds: chlorothalonil, dichlofluanid, dichlorophen, flusulfamide, hexachlorobenzene, pencycuron, pentachlorophenol and its salts, phthalide, quintozene, thiophanate methyl, tolylfluanid, N-(4-chloro-2-nitrophenyl)-N-ethyl-4-methylbenzenesulfonamide;
   inorganic active compounds: phosphorous acid and its salts, Bordeaux mixture, copper salts, such as, for example, copper acetate, copper hydroxide, copper oxy-chloride, basic copper sulfate, sulfur;
   others: biphenyl, bronopol, cyflufenamid, cymoxanil, diphenylamine, metrafenone, mildiomycin, oxine-copper, prohexadione-calcium, spiroxamine, tolylfluanid, N-(cyclopropylmethoxyimino-(6-difluoromethoxy-2,3-difluorophenyl)methyl)-2-phenylacetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)phenyl)-N-ethyl-N-methylformamidine, N′-(5-difluoromethyl-2-methyl-4-(3-trimethylsilanylpropoxy)phenyl)-N-ethyl-N-methylformamidine, methyl N-(1,2,3,4-tetrahydronaphthalen-1-yl)-2-{1-[2-(5-methyl-3-trifluoromethylpyrazol-1-yl)acetyl]piperidin-4-yl}thiazole-4-carboxamide, methyl (R)—N-(1,2,3,4-tetrahydronaphthalen-1-yl)-2-{1-[2-(5-methyl-3-trifluoromethylpyrazol-1-yl)acetyl]-piperidin-4-yl}thiazole-4-carboxamide, 6-tert-butyl-8-fluoro-2,3-dimethylquinolin-4-yl acetate, 6-tert-butyl-8-fluoro-2,3-dimethylquinolin-4-yl methoxyacetate, N-methyl-2-{1-[2-(5-methyl-3-trifluoromethyl-1H-pyrazol-1-yl)acetyl]piperidin-4-yl}-N-[(1R)-1,2,3,4-tetrahydronaphthalen-1-yl]-4-thiazolecarboxamide;
G) growth regulators
   abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfid, indole-3-acetic acid, maleic hydrazide, mefluidide, mepiquat (mepiquat chloride), metconazole, naphthalene acetic acid, N-6-benzyladenine, paclobutrazole, prohexadione (prohexadione-calcium), prohydrojasmon, thidiazuron, triapenthenol, tributyl phosphorotrithioate, 2,3,5-triiodobenzoic acid, trinexapac-ethyl and uniconazole;
H) herbicides
   acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
   amino acid analogues: bilanafos, glyphosate, glufosinate, sulfosate;
   aryloxyphenoxypropionates: clodinafop, cyhalofop-butyl, fenoxaprop, fluazifop, haloxyfop, metamifop, propaquizafop, quizalofop, quizalofop-p-tefuryl;
   bipyridyls: diquat, paraquat;
   carbamates and thiocarbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
   cyclohexanediones: butroxydim, clethodim, cycloxydim, profoxydim, sethoxydim, tepraloxydim, tralkoxydim;
   dinitroanilines: benfluralin, ethalfluralin, oryzalin, pendimethalin, prodiamine, trifluralin;
   diphenyl ethers: acifluorfen, aclonifen, bifenox, diclofop, ethoxyfen, fomesafen, lactofen, oxyfluorfen;
   hydroxybenzonitriles: bromoxynil, dichlobenil, ioxynil;
   imidazolinones: imazamethabenz, imazamox, imazapic, imazapyr, imazaquin, imazethapyr;
   phenoxyacetic acids: clomeprop, 2,4-dichlorophenoxyacetic acid (2,4-D), 2,4-DB, dichiorprop, MCPA, MCPA-thioethyl, MCPB, mecoprop;
   pyrazines: chloridazone, flufenpyr-ethyl, fluthiacet, norflurazone, pyridate;
   pyridines: aminopyralid, clopyralid, diflufenican, dithiopyr, fluridone, fluoroxypyr, picloram, picolinafen, thiazopyr;
   sulfonylureas: amidosulfuron, azimsulfuron, bensulfuron, chlorimuron-ethyl, chlorsulfuron, cinosulfuron, cyclosulfamuron, ethoxysulfuron, flazasulfuron, fluce-tosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosul-furon, pyrazosulfuron, rimsulfuron, sulfometuron, sulfosulfuron, thifensulfuron, tria-sulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxypyrimidin-2-yl)urea;
   triazines: ametryn, atrazine, cyanazine, dimethametryn, ethiozine, hexazinone, meta-mitron, metribuzin, prometryn, simazine, terbuthylazine, terbutryn, triaziflam;
   ureas: chlorotoluron, daimuron, diuron, fluometuron, isoproturon, linuron, methabenzthiazuron, tebuthiuron;
   other inhibitors of acetolactate synthase: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
   others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methylarsenic acid, naptalam, oxadiargyl, oxadiazon, oxaziclomefone, pentoxazone, pinoxaden, pyraclonil, pyraflufen-ethyl, pyrasulfotol, pyrazoxyfen, pyrazolynate, quinoclamine, saflufenacil, sulcotrion, sulfentrazone, terbacil, tefuryltrione, ternbotrione, thiencarbazone, topramezone, 4-hydroxy-3-[2-(2-methoxyethoxymethyl)-6-trifluoromethylpyridine-3-carbonyl]bicyclo[3.2.1]oct-3-en-2-one, ethyl (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetate, methyl 6-amino-5-chloro-2-cyclopropylpyrimidine-4-carboxylate, 6-chloro-3-(2-cyclopropyl-6-methylphenoxy)-pyridazin-4-ol, 4-amino-3-chloro-6-(4-chlorophenyl)-5-fluoropyridine-2-carboxylic acid, methyl 4-amino-3-chloro-6-(4-chloro-2-fluoro-3-methoxyphenyl)pyridine-2-carboxylate and methyl 4-amino-3-chloro-6-(4-chloro-3-dimethylamino-2-fluorophenyl)pyridine-2-carboxylate;
I) insecticides
   organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
   carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
   pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin,
   inhibitors of insect growth: a) chitin synthesis inhibitors: benzoylureas: chlorfluazuron, cyromazine, diflubenzuron, flucycloxuron, flufenoxuron, hexaflumuron, lufenuron, novaluron, teflubenzuron, triflumuron; buprofezin, diofenolan, hexythiazox, etoxazole, clofentazin; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate;
   nicotine receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl-[1,3,5]triazinane;
   GABA antagonists: endosulfan, ethiprol, fipronil, vaniliprol, pyrafluprol, pyriprol, 5-amino-1-(2,6-dichloro-4-methylphenyl)-4-sulfinamoyl-1H-pyrazole-3-thiocarboxamide;
   macrocyclic lactones: abamectin, emamectin, milbemectin, lepimectin, spinosad, spinetoram;
   mitochondrial electron transport chain inhibitor (METI) I acaricides: fenazaquin, pyridaben, tebufenpyrad, tolfenpyrad, flufenerim;
   METI II and III substances: acequinocyl, fluacyprim, hydramethylnon;
   decouplers: chlorfenapyr;
   inhibitors of oxidative phosphorylation: cyhexatin, diafenthiuron, fenbutatin oxide, propargite;
   insect molting inhibitors: cryomazine;
   mixed function oxidase inhibitors: piperonyl butoxide;
   sodium channel blockers: indoxacarb, metaflumizone;
   others: benclothiaz, bifenazate, cartap, flonicamid, pyridalyl, pymetrozine, sulfur, thiocyclam, flubendiamid, chlorantraniliprol, cyazypyr (HGW86); cyenopyrafen, flupyrazofos, cyflumetofen, amidoflumet, imicyafos, bistrifluoron, and pyrifluquinazon.

The present invention relates in particular also to fungicidal compositions which comprise at least one compound of the general formula I and at least one further crop protection agent, in particular at least one fungicidal active compound, for example one or more, for example 1 or 2, active compounds of groups A) to F) mentioned above and, optionally, one or more agriculturally suitable carriers. With a view to reducing the application rates, these mixtures are of interest, since many show, at a reduced total amount of active compounds applied, an improved activity against harmful fungi, in particular for certain indications. By simultaneous joint or separate application of compound(s) I with at least one active compound of groups A) to I), the fungicidal activity can be increased in a superadditive manner.

In the sense of the present application, joint application means that the at least one compound I and the at least one further active compound are present simultaneously at the site of action (i.e. the plant-damaging fungi to be controlled and their habitat, such as infected plants, plant propagation materials, in particular seed, soils, materials or spaces and also plants, plant propagation materials, in particular seed, soils, materials or spaces to be protected against fungal attack) in an amount sufficient for an effective control of fungal growth. This can be achieved by applying the compounds I and at least one further active compound jointly in a joint active compound preparation or in at least two separate active compound preparations simultaneously, or by applying the active compounds successively to the site of action, the interval between the individual active compound applications being chosen such that the active compound applied first is, at the time of application of the further active compound(s), present at the site of action in a sufficient amount. The order in which the active compounds are applied is of minor importance.

In binary mixtures, i.e. compositions according to the invention comprising a compound I and a further active compound, for example an active compound of groups A) to I), the weight ratio of compound I to the 1st further active compound depends on the properties of the active compounds in question; usually, it is in the range of from 1:100 to 100:1, frequently in the range of from 1:50 to 50:1, preferably in the range of from 1:20 to 20:1, particularly preferably in the range of from 1:10 to 10:1, especially in the range of from 1:3 to 3:1.

In ternary mixtures, i.e. compositions according to the invention comprising an active compound I and a 1st further active compound and a 2nd further active compound, for example two different active compounds from groups A) to I), the weight ratio of compound Ito the 1st further active compound depends on the properties of the respective active compounds; preferably, it is in the range of from 1:50 to 50:1 and in particular in the range of from 1:10 to 10:1. The weight ratio of compound I to the 2nd further active compound is preferably in the range of from 1:50 to 50:1, in particular in the range of from 1:10 to 10:1. The weight ratio of 1st further active compound to 2nd further active compound is preferably in the range of from 1:50 to 50:1, in particular in the range of from 1:10 to 10:1.

The components of the composition according to the invention can be packaged and used individually or as a ready-mix or as a kit of parts.

In one embodiment of the invention, the kits may comprise one or more, and even all, components used for preparing an agrochemical composition according to the invention. For example, these kits may comprise one or more fungicide components and/or an adjuvant component and/or an insecticide component and/or a growth regulator component and/or a herbicide. One or more components may be present combined or preformulated with one another. In the embodiments where more than two components are provided in a kit, the components can be combined with one another and be packaged in a single container, such as a vessel, a bottle, a tin, a bag, a sack or canister. In other embodiments, two or more components of a kit may be packaged separately, i.e. not preformulated or mixed. Kits may comprise one or more separate containers, such as vessels, bottles, tins, bags, sacks or canisters, each container comprising a separate component of the agrochemical composition. The components of the composition according to the invention can be packaged and used individually or as a ready-mix or as a kit of parts. In both forms, a component may be used separately or together with the other components or as a part of a kit of parts according to the invention for preparing the mixture according to the invention.

The user uses the composition according to the invention usually for use in a predosage device, a knapsack sprayer, a spray tank or a spray plane. Here, the agrochemical composition is diluted with water and/or buffer to the desired application concentration, with further auxiliaries being added, if required, thus giving the ready-to-use spray liquor or the agrochemical composition according to the invention. Usually, from 50 to 500 liters of the ready-to-use spray liquor are applied per hectare of agricultural utilized area, preferably from 100 to 400 liters.

According to one embodiment, the user may himself mix individual components, such as, for example, parts of a kit or a two- or three-component mixture of the composition according to the invention in a spray tank and, if required, add further auxiliaries (tank mix).

In a further embodiment, the user may mix both individual components of the composition according to the invention and partially pre-mixed components, for example components comprising compounds I and/or active compounds from groups A) to I), in a spray tank and, if required, add further auxiliaries (tank mix).

In a further embodiment, the user may use both individual components of the composition according to the invention and partially pre-mixed components, for example components comprising compounds I and/or active compounds from groups A) to I), jointly (for example as a tank mix) or in succession.

Preference is given to compositions of a compound I (component 1) with at least one active compound from group A) (component 2) of the strobilurins and in particular selected from the group consisting of azoxystrobin, dimoxystrobin, fluoxastrobin, kresoxim-methyl, orysastrobin, picoxystrobin, pyraclostrobin and trifloxystrobin.

Preference is also given to compositions of a compound I (component 1) with at least one active compound selected from group B) (component 2) of the carboxamides and in particular selected from the group consisting of bixafen, boscalid, isopyrazam, fluopyram, penflufen, penthiopyrad, sedaxane, fenhexamid, metalaxyl, mefenoxam, ofurace, dimethomorph, flumorph, fluopicolid (picobenzamid), zoxamide, carpropamid, mandipropamid and N-(3′,4′,5′-trifluorobiphenyl-2-yl)-3-difluoromethyl-1-methyl-1H-pyrazole-4-carboxamide.

Preference is also given to compositions of a compound I (component 1) with at least one active compound selected from group C) (component 2) of the azoles and in particular selected from the group consisting of cyproconazole, difenoconazole, epoxiconazole, fluquinconazole, flusilazole, flutriafole, metconazole, myclobutanil, penconazole, propiconazole, prothioconazole, triadimefon, triadimenol, tebuconazole, tetraconazole, triticonazole, prochloraz, cyazofamid, benomyl, carbendazim and ethaboxam.

Preference is also given to compositions of a compound I (component 1) with at least one active compound selected from group D) (component 2) of the nitrogenous heterocyclyl compounds and in particular selected from the group consisting of fluazinam, cyprodinil, fenarimol, mepanipyrim, pyrimethanil, triforin, fludioxonil, fodemorph, fenpropimorph, tridemorph, fenpropidin, iprodion, vinclozolin, famoxadone, fenamidone, probenazole, proquinazid, acibenzolar-s-methyl, captafol, folpet, fenoxanil, quinoxyfen and 5-ethyl-6-octyl-[1,2,4]-triazolo-[1,5-a]pyrimidin-7-ylamine.

Preference is also given to compositions of a compound I (component I) with at least one active compound selected from group E) (component 2) of the carbamates and in particular selected from the group consisting of mancozeb, metiram, propineb, thiram, iprovalicarb, benthiavalicarb and propamocarb.

Preference is also given to compositions of a compound I (component I) with at least one active compound selected from the fungicides of group F) (component 2) and in particular selected from the group consisting of dithianon, fentin salts, such as fentin acetate, fosetyl, fosetyl-aluminum, H₃PO₃ and salts thereof, chlorothalonil, dichlofluanid, thiophanate-methyl, copper acetate, copper hydroxide, copper oxychloride, copper sulfate, sulfur, cymoxanil, metrafenone, spiroxamine and N-methyl-2-{1-[(5-methyl-3-trifluoromethyl-1H-pyrazol-1-yOacetyl]piperidin-4-yl}-N-[(1R)-1,2,3,4-tetrahydronaphthalen-1-yl]-4-thiazolecarboxamide.

Accordingly, the present invention furthermore relates to compositions of a compound I (component 1) with a further active compound (component 2), the latter being selected from rows B-1 to B-347 in the column “component 2” of Table B.

A further embodiment of the invention relates to the compositions B-1 to B-347 listed in Table B, where a row of Table B corresponds in each case to an agrochemical composition comprising one of the compounds of the formula I individualized in the present description (component 1) and the respective further active compound from the groups A) to I) (component 2) stated in the row in question. According to one embodiment, the component 1 corresponds to one of the compounds I individualized in Tables 1a to 351a. The active compounds in the described compositions are in each case preferably present in synergistically active amounts.

TABLE B
Active compound composition, comprising an individualized compound I and a
further active compound from the groups A) to I)

| Row | Component 1 | Component 2 | |
|---|---|---|---|
| B-1 | an individualized compound I | azoxystrobin | |
| B-2 | an individualized compound I | dimoxystrobin | |
| B-3 | an individualized compound I | enestroburin | |
| B-4 | an individualized compound I | fluoxastrobin | |
| B-5 | an individualized compound I | kresoxim-methyl | |
| B-6 | an individualized compound I | metominostrobin | |
| B-7 | an individualized compound I | orysastrobin | |
| B-8 | an individualized compound I | picoxystrobin | |
| B-9 | an individualized compound I | pyraclostrobin | |
| B-10 | an individualized compound I | pyribencarb | |
| B-11 | an individualized compound I | trifloxystrobin | |
| B-12 | an individualized compound I | 2-(2-(6-(3-chloro-2-methylphenoxy)- | |
| | | 5-fluoropyrimidin-4-yloxy)phenyl)-2- | |
| | | methoxyimino-N-methylacetamide | |
| B-13 | an individualized compound I | 2-(o-((2,5-dimethylphenyloxy- | |
| | | methylene)phenyl)-3-methoxyacrylic acid | |
| | | methyl ester | |
| B-14 | an individualized compound I | 3-methoxy-2-(2-(N-(4-methoxyphenyl)- | |
| | | cyclopropanecarboximidoylsulfanyl- | |
| | | methyl)phenyl)acrylic acid methyl ester | |
| B-15 | an individualized compound I | 2-(2-(3-(2,6-dichlorophenyl)-1- | |
| | | methylallylideneaminooxymethyl)phenyl)- | |
| | | 2-methoxyimino-N-methylacetamide | |
| B-16 | an individualized compound I | benalaxyl | |
| B-17 | an individualized compound I | benalaxyl-M | |
| B-18 | an individualized compound I | benodanil | |
| B-19 | an individualized compound I | bixafen | |
| B-20 | an individualized compound I | boscalid | |
| B-21 | an individualized compound I | carboxin | |
| B-22 | an individualized compound I | fenfuram | |
| B-23 | an individualized compound I | fenhexamid | |
| B-24 | an individualized compound I | flutolanil | |
| B-25 | an individualized compound I | furametpyr | |
| B-26 | an individualized compound I | isopyrazam | |
| B-27 | an individualized compound I | isotianil | |
| B-28 | an individualized compound I | kiralaxyl | |
| B-29 | an individualized compound I | mepronil | |
| B-30 | an individualized compound I | metalaxyl | |
| B-31 | an individualized compound I | metalaxyl-M | |
| B-32 | an individualized compound I | ofurace | |
| B-33 | an individualized compound I | oxadixyl | |
| B-34 | an individualized compound I | oxycarboxin | |
| B-35 | an individualized compound I | penflufen | |
| B-36 | an individualized compound I | penthiopyrad | |
| B-37 | an individualized compound I | sedaxane | |
| B-38 | an individualized compound I | tecloftalam | |
| B-39 | an individualized compound I | thifluzamide | |
| B-40 | an individualized compound I | tiadinil | |
| B-41 | an individualized compound I | 2-amino-4-methylthiazole-5-carboxanilide | |
| B-42 | an individualized compound I | 2-chloro-N-(1,1,3-trimethylindan-4-yl)- | |
| | | nicotinamide | |
| B-43 | an individualized compound I | N-(3′,4′,5′-trifluorobiphenyl-2-yl)-3- | |
| | | difluoromethyl-1-methyl-1H-pyrazole-4- | |
| | | carboxamide | |
| B-44 | an individualized compound I | N-(4′-trifluoromethylthiobiphenyl-2-yl)-3- | |
| | | difluoromethyl-1-methyl-1H-pyrazole-4- | |
| | | carboxamide | |
| B-45 | an individualized compound I | N-(2-(1,3,3-trimethylbutyl)phenyl)-1,3- | |
| | | dimethyl-5-fluoro-1H-pyrazole-4- | |
| | | carboxamide | |
| B-46 | an individualized compound I | dimethomorph | |
| B-47 | an individualized compound I | flumorph | |
| B-48 | an individualized compound I | pyrimorph | |
| B-49 | an individualized compound I | flumetover | |
| B-50 | an individualized compound I | fluopicolide | |
| B-51 | an individualized compound I | fluopyram | |
| B-52 | an individualized compound I | zoxamide | |
| B-53 | an individualized compound I | N-(3-ethyl-3,5,5-trimethylcyclohexyl)- | |
| | | 3-formylamino-2-hydroxybenzamide | |
| B-54 | an individualized compound I | carpropamid | |
| B-55 | an individualized compound I | diclocymet | |
| B-56 | an individualized compound I | mandipropamid | |
| B-57 | an individualized compound I | oxytetracyclin | |
| B-58 | an individualized compound I | silthiofam | |
| B-59 | an individualized compound I | N-(6-methoxypyridin-3-yl)cyclopropane- | |
| | | carboxamide | |
| B-60 | an individualized compound I | azaconazole | |
| B-61 | an individualized compound I | bitertanol | |
| B-62 | an individualized compound I | bromuconazole | |
| B-63 | an individualized compound I | cyproconazole | |
| B-64 | an individualized compound I | difenoconazole | |
| B-65 | an individualized compound I | diniconazole | |
| B-66 | an individualized compound I | diniconazole-M | |
| B-67 | an individualized compound I | epoxiconazole | |
| B-68 | an individualized compound I | fenbuconazole | |
| B-69 | an individualized compound I | fluquinconazole | |
| B-70 | an individualized compound I | flusilazole | |
| B-71 | an individualized compound I | flutriafol | |
| B-72 | an individualized compound I | hexaconazole | |
| B-73 | an individualized compound I | imibenconazole | |
| B-74 | an individualized compound I | ipconazole | |
| B-75 | an individualized compound I | metconazole | |
| B-76 | an individualized compound I | myclobutanil | |
| B-77 | an individualized compound I | oxpoconazole | |
| B-78 | an individualized compound I | paclobutrazole | |
| B-79 | an individualized compound I | penconazole | |
| B-80 | an individualized compound I | propiconazole | |
| B-81 | an individualized compound I | prothioconazole | |
| B-82 | an individualized compound I | simeconazole | |
| B-83 | an individualized compound I | tebuconazole | |
| B-84 | an individualized compound I | tetraconazole | |
| B-85 | an individualized compound I | triadimefon | |
| B-86 | an individualized compound I | triadimenol | |
| B-87 | an individualized compound I | triticonazole | |
| B-88 | an individualized compound I | uniconazole | |
| B-89 | an individualized compound I | 1-(4-chlorophenyl)-2-([1,2,4]triazol-1-yl)- | |
| | | cycloheptanol | |
| B-90 | an individualized compound I | cyazofamid | |
| B-91 | an individualized compound I | imazalil | |
| B-92 | an individualized compound I | imazalil-sulfate | |
| B-93 | an individualized compound I | pefurazoate | |
| B-94 | an individualized compound I | prochloraz | |
| B-95 | an individualized compound I | triflumizole | |
| B-96 | an individualized compound I | benomyl | |
| B-97 | an individualized compound I | carbendazim | |
| B-98 | an individualized compound I | fuberidazole | |
| B-99 | an individualized compound I | thiabendazole | |
| B-100 | an individualized compound I | ethaboxam | |
| B-101 | an individualized compound I | etridiazole | |
| B-102 | an individualized compound I | hymexazole | |
| B-103 | an individualized compound I | 2-(4-chlorophenyl)-N-[4- | |
| | | (3,4-dimethoxyphenyl)isoxazol-5-yl]-2-prop-2- | |
| | | ynyloxyacetamide | |
| B-104 | an individualized compound I | fluazinam | |
| B-105 | an individualized compound I | pyrifenox | |
| B-106 | an individualized compound I | 3-[5-(4-chlorophenyl)-2,3-dimethyl- | |
| | | isoxazolidin-3-yl]pyridine | |
| B-107 | an individualized compound I | 3-[5-(4-methylphenyl)-2,3- | |
| | | dimethylisoxazolidin-3-yl]pyridine | |
| B-108 | an individualized compound I | 2,3,5,6-tetrachloro-4-methanesulfonyl- | |
| | | pyridine | |
| B-109 | an individualized compound I | 3,4,5-trichloropyridine-2,6-dicarbonitrile | |
| B-110 | an individualized compound I | N-(1-(5-bromo-3-chloropyridin-2-yl)ethyl)-2,4- | |
| | | dichloronicotinamide | |
| B-111 | an individualized compound I | N-((5-bromo-3-chloropyridin-2-yl)methyl)- | |
| | | 2,4-dichloronicotinamide | |
| B-112 | an individualized compound I | bupirimate | |
| B-113 | an individualized compound I | cyprodinil | |
| B-114 | an individualized compound I | diflumetorim | |
| B-115 | an individualized compound I | fenarimol | |
| B-116 | an individualized compound I | ferimzone | |
| B-117 | an individualized compound I | mepanipyrim | |
| B-118 | an individualized compound I | nitrapyrin | |
| B-119 | an individualized compound I | nuarimol | |
| B-120 | an individualized compound I | pyrimethanil | |
| B-121 | an individualized compound I | triforine | |
| B-122 | an individualized compound I | fenpiclonil | |
| B-123 | an individualized compound I | fludioxonil | |
| B-124 | an individualized compound I | aldimorph | |
| B-125 | an individualized compound I | dodemorph | |
| B-126 | an individualized compound I | dodemorph acetate | |
| B-127 | an individualized compound I | fenpropimorph | |
| B-128 | an individualized compound I | tridemorph | |
| B-129 | an individualized compound I | fenpropidin | |
| B-130 | an individualized compound I | fluoroimide | |
| B-131 | an individualized compound I | iprodione | |
| B-132 | an individualized compound I | procymidone | |
| B-133 | an individualized compound I | vinclozolin | |
| B-134 | an individualized compound I | famoxadone | |
| B-135 | an individualized compound I | fenamidone | |
| B-136 | an individualized compound I | flutianil | |
| B-137 | an individualized compound I | octhilinone | |
| B-138 | an individualized compound I | probenazole | |
| B-139 | an individualized compound I | S-allyl 5-amino-2-isopropyl-4- | |
| | | orthotolylpyrazol-3-one-1-thiocarboxylate | |
| B-140 | an individualized compound I | acibenzolar-S-methyl | |
| B-141 | an individualized compound I | amisulbrom | |
| B-142 | an individualized compound I | anilazine | |
| B-143 | an individualized compound I | blasticidin-S | |
| B-144 | an individualized compound I | captafol | |
| B-145 | an individualized compound I | captan | |
| B-146 | an individualized compound I | chinomethionate | |
| B-147 | an individualized compound I | dazomet | |
| B-148 | an individualized compound I | debacarb | |
| B-149 | an individualized compound I | diclomezine | |
| B-150 | an individualized compound I | difenzoquat | |
| B-151 | an individualized compound I | difenzoquat methylsulfate | |
| B-152 | an individualized compound I | fenoxanil | |
| B-153 | an individualized compound I | folpet | |
| B-154 | an individualized compound I | oxolinic acid | |
| B-155 | an individualized compound I | piperalin | |
| B-156 | an individualized compound I | proquinazid | |
| B-157 | an individualized compound I | pyroquilon | |
| B-158 | an individualized compound I | quinoxyfen | |
| B-159 | an individualized compound I | triazoxide | |
| B-160 | an individualized compound I | tricyclazole | |
| B-161 | an individualized compound I | 2-butoxy-6-iodo-3-propylchromen-4-one | |
| B-162 | an individualized compound I | 5-chloro-1-(4,6-dimethoxypyrimidin-2-yl)- | |
| | | 2-methyl-1H-benzimidazole | |
| B-163 | an individualized compound I | 5-chloro-7-(4-methylpiperidin-1-yl)- | |
| | | 6-(2,4,6-trifluorophenyl)-[1,2,4]triazolo- | |
| | | [1,5-a]pyrimidine | |
| B-164 | an individualized compound I | 5-ethyl-6-octyl-[1,2,4]triazolo[1,5- | |
| | | a]pyrimidin-7-ylamine | |
| B-165 | an individualized compound I | ferbam | |
| B-166 | an individualized compound I | mancozeb | |
| B-167 | an individualized compound I | maneb | |
| B-168 | an individualized compound I | metam | |
| B-169 | an individualized compound I | methasulfocarb | |
| B-170 | an individualized compound I | metiram | |
| B-171 | an individualized compound I | propineb | |
| B-172 | an individualized compound I | thiram | |
| B-173 | an individualized compound I | zineb | |
| B-174 | an individualized compound I | ziram | |
| B-175 | an individualized compound I | diethofencarb | |
| B-176 | an individualized compound I | benthiavalicarb | |
| B-177 | an individualized compound I | iprovalicarb | |
| B-178 | an individualized compound I | propamocarb | |
| B-179 | an individualized compound I | propamocarb hydrochloride | |
| B-180 | an individualized compound I | valiphenal | |
| B-181 | an individualized compound I | 4-fluorophenyl N-(1-(1-(4-cyanophenyl)- | |
| | | ethanesulfonyl)but-2-yl)carbamate | |
| B-182 | an individualized compound I | dodine | |
| B-183 | an individualized compound I | dodine free base | |
| B-184 | an individualized compound I | guazatine | |
| B-185 | an individualized compound I | guazatine acetate | |
| B-186 | an individualized compound I | iminoctadine | |
| B-187 | an individualized compound I | iminoctadine triacetate | |
| B-188 | an individualized compound I | iminoctadine tris(albesilate) | |
| B-189 | an individualized compound I | kasugamycin | |
| B-190 | an individualized compound I | kasugamycin hydrochloride hydrate | |
| B-191 | an individualized compound I | polyoxin | |
| B-192 | an individualized compound I | streptomycin | |
| B-193 | an individualized compound I | validamycin A | |
| B-194 | an individualized compound I | binapacryl | |
| B-195 | an individualized compound I | dicloran | |
| B-196 | an individualized compound I | dinobuton | |
| B-197 | an individualized compound I | dinocap | |
| B-198 | an individualized compound I | nitrothal-isopropyl | |
| B-199 | an individualized compound I | tecnazen | |
| B-200 | an individualized compound I | fentin salts | |
| B-201 | an individualized compound I | dithianon | |
| B-202 | an individualized compound I | isoprothiolane | |
| B-203 | an individualized compound I | edifenphos | |
| B-204 | an individualized compound I | fosetyl, fosetyl aluminum | |
| B-205 | an individualized compound I | iprobenfos | |
| B-206 | an individualized compound I | phosphorous acid and derivatives | |
| B-207 | an individualized compound I | pyrazophos | |
| B-208 | an individualized compound I | tolclofos-methyl | |
| B-209 | an individualized compound I | chlorthalonil | |
| B-210 | an individualized compound I | dichlofluanid | |
| B-211 | an individualized compound I | dichlorophene | |
| B-212 | an individualized compound I | flusulfamide | |
| B-213 | an individualized compound I | hexachlorobenzene | |
| B-214 | an individualized compound I | pencycuron | |
| B-215 | an individualized compound I | pentachlorophenol and salts | |
| B-216 | an individualized compound I | phthalide | |
| B-217 | an individualized compound I | quintozene | |
| B-218 | an individualized compound I | thiophanate methyl | |
| B-219 | an individualized compound I | tolylfluanid | |
| B-220 | an individualized compound I | N-(4-chloro-2-nitrophenyl)-N-ethyl- | |
| | | 4-methylbenzenesulfonamide | |
| B-221 | an individualized compound I | Bordeaux mixture | |
| B-222 | an individualized compound I | copper acetate | |
| B-223 | an individualized compound I | copper hydroxide | |
| B-224 | an individualized compound I | copper oxychloride | |
| B-225 | an individualized compound I | basic copper sulfate | |
| B-226 | an individualized compound I | sulfur | |
| B-227 | an individualized compound I | biphenyl | |
| B-228 | an individualized compound I | bronopol | |
| B-229 | an individualized compound I | cyflufenamid | |
| B-230 | an individualized compound I | cymoxanil | |
| B-231 | an individualized compound I | diphenylamine | |
| B-232 | an individualized compound I | metrafenone | |
| B-233 | an individualized compound I | mildiomycin | |
| B-234 | an individualized compound I | oxine-copper | |
| B-235 | an individualized compound I | prohexadione-calcium | |
| B-236 | an individualized compound I | spiroxamine | |
| B-237 | an individualized compound I | tolylfluanid | |
| B-238 | an individualized compound I | N-(cyclopropylmethoxyimino-(6-difluoro- | |
| | | methoxy-2,3-difluorophenyl)methyl)- | |
| | | 2-phenylacetamide | |
| B-239 | an individualized compound I | N′-(4-(4-chloro-3-trifluoromethylphenoxy)- | |
| | | 2,5-dimethylphenyl)-N-ethyl-N-methyl- | |
| | | formamidine | |
| B-240 | an individualized compound I | N′-(4-(4-fluoro-3-trifluoromethylphenoxy)- | |
| | | 2,5-dimethylphenyl)-N-ethyl-N-methyl- | |
| | | formamidine | |
| B-241 | an individualized compound I | N′-(2-methyl-5-trifluoromethyl-4-(3-tri- | |
| | | methylsilanylpropoxy)phenyl)-N-ethyl- | |
| | | N-methylformamidine | |
| B-242 | an individualized compound I | N′-(5-difluoromethyl-2-methyl-4-(3-tri- | |
| | | methylsilanylpropoxy)phenyl)-N-ethyl- | |
| | | N-methylformamidine | |
| B-243 | an individualized compound I | methyl N-(1,2,3,4-tetrahydronaphthalen- | |
| | | 1-yl)-2-{1-[2-(5-methyl-3-trifluoromethyl- | |
| | | pyrazol-1-yl)acetyl]piperidin-4-yl}thiazole- | |
| | | 4-carboxamide | |
| B-244 | an individualized compound I | methyl N—(R)-1,2,3,4- | |
| | | tetrahydronaphthalen-1-yl)-2-{1-[2-(5- | |
| | | methyl-3-trifluoromethyl-pyrazol-1- | |
| | | yl)acetyl]piperidin-4-yl}thiazole-4- | |
| | | carboxamide | |
| B-245 | an individualized compound I | 6-tert-butyl-8-fluoro-2,3-dimethylquinolin- | |
| | | 4-yl acetate | |
| B-246 | an individualized compound I | 6-tert-butyl-8-fluoro-2,3-dimethylquinolin- | |
| | | 4-yl methoxyacetate | |
| B-247 | an individualized compound I | N-methyl-2-{1-[(5-methyl-3-trifluoro- | |
| | | methyl-1H-pyrazol-1-yl)acetyl]piperidin-4- | |
| | | yl}-N-[(1R)-1,2,3,4-tetrahydronaphthalen- | |
| | | 1-yl]-4-thiazolecarboxamide | |
| B-248 | an individualized compound I | carbaryl | |
| B-249 | an individualized compound I | carbofuran | |
| B-250 | an individualized compound I | carbosulfan | |
| B-251 | an individualized compound I | methomylthiodicarb | |
| B-252 | an individualized compound I | bifenthrin | |
| B-253 | an individualized compound I | cyfluthrin | |
| B-254 | an individualized compound I | cypermethrin | |
| B-255 | an individualized compound I | alpha-cypermethrin | |
| B-256 | an individualized compound I | zeta-cypermethrin | |
| B-257 | an individualized compound I | deltamethrin | |
| B-258 | an individualized compound I | esfenvalerate | |
| B-259 | an individualized compound I | A-367 | lambda-cyhalothrin |
| B-260 | an individualized compound I | A-368 | permethrin |
| B-261 | an individualized compound I | A-369 | tefluthrin |
| B-262 | an individualized compound I | A-370 | diflubenzuron |
| B-263 | an individualized compound I | A-371 | flufenoxuron |
| B-264 | an individualized compound I | A-372 | lufenuron |
| B-265 | an individualized compound I | A-373 | teflubenzuron |
| B-266 | an individualized compound I | A-374 | spirotetramate |
| B-267 | an individualized compound I | A-375 | clothianidin |
| B-268 | an individualized compound I | A-376 | dinotefuran |
| B-269 | an individualized compound I | A-377 | imidacloprid |
| B-270 | an individualized compound I | A-378 | thiamethoxam |
| B-271 | an individualized compound I | A-379 | acetamiprid |
| B-272 | an individualized compound I | A-380 | thiacloprid |
| B-273 | an individualized compound I | A-381 | endosulfan |
| B-274 | an individualized compound I | A-382 | fipronil |
| B-275 | an individualized compound I | A-383 | abamectin |
| B-276 | an individualized compound I | A-384 | emamectin |
| B-277 | an individualized compound I | A-385 | spinosad |
| B-278 | an individualized compound I | A-386 | spinetoram |
| B-279 | an individualized compound I | A-387 | hydramethylnon |
| B-280 | an individualized compound I | A-388 | chlorfenapyr |
| B-281 | an individualized compound I | A-389 | fenbutatin oxide |
| B-282 | an individualized compound I | A-390 | indoxacarb |
| B-283 | an individualized compound I | A-391 | metaflumizone |
| B-284 | an individualized compound I | A-392 | flonicamid |

| | | |
|---|---|---|
| B-285 | an individualized compound I | lubendiamid |
| B-286 | an individualized compound I | chlorantraniliprol |
| B-287 | an individualized compound I | cyazypyr (HGW86) |
| B-288 | an individualized compound I | cyflumetofen |
| B-289 | an individualized compound I | acetochlor |
| B-290 | an individualized compound I | dimethenamid |
| B-291 | an individualized compound I | metolachlor |
| B-292 | an individualized compound I | metazachlor |
| B-293 | an individualized compound I | glyphosate |
| B-294 | an individualized compound I | glufosinate |
| B-295 | an individualized compound I | sulfosate |
| B-296 | an individualized compound I | clodinafop |
| B-297 | an individualized compound I | fenoxaprop |
| B-298 | an individualized compound I | fluazifop |
| B-299 | an individualized compound I | haloxyfop |
| B-300 | an individualized compound I | paraquat |
| B-301 | an individualized compound I | phenmedipham |
| B-302 | an individualized compound I | clethodim |
| B-303 | an individualized compound I | cycloxydim |
| B-304 | an individualized compound I | profoxydim |
| B-305 | an individualized compound I | sethoxydim |
| B-306 | an individualized compound I | tepraloxydim |
| B-307 | an individualized compound I | pendimethalin |
| B-308 | an individualized compound I | prodiamine |
| B-309 | an individualized compound I | trifluralin |
| B-310 | an individualized compound I | acifluorfen |
| B-311 | an individualized compound I | bromoxynil |
| B-312 | an individualized compound I | imazamethabenz |
| B-313 | an individualized compound I | imazamox |
| B-314 | an individualized compound I | imazapic |
| B-315 | an individualized compound I | imazapyr |
| B-316 | an individualized compound I | imazaquin |
| B-317 | an individualized compound I | imazethapyr |
| B-318 | an individualized compound I | 2,4-dichlorophenoxyacetic acid (2,4-D) |
| B-319 | an individualized compound I | chloridazon |
| B-320 | an individualized compound I | clopyralid |
| B-321 | an individualized compound I | fluroxypyr |
| B-322 | an individualized compound I | picloram |
| B-323 | an individualized compound I | picolinafen |
| B-324 | an individualized compound I | bensulfuron |
| B-325 | an individualized compound I | chlorimuron-ethyl |
| B-326 | an individualized compound I | cyclosulfamuron |
| B-327 | an individualized compound I | iodosulfuron |
| B-328 | an individualized compound I | mesosulfuron |
| B-329 | an individualized compound I | metsulfuron-methyl |
| B-330 | an individualized compound I | nicosulfuron |
| B-331 | an individualized compound I | rimsulfuron |
| B-332 | an individualized compound I | triflusulfuron |
| B-333 | an individualized compound I | atrazine |
| B-334 | an individualized compound I | hexazinone |
| B-335 | an individualized compound I | diuron |
| B-336 | an individualized compound I | florasulam |
| B-337 | an individualized compound I | pyroxasulfon |
| B-338 | an individualized compound I | bentazone |
| B-339 | an individualized compound I | cinidon-ethyl |
| B-340 | an individualized compound I | cinmethylin |
| B-341 | an individualized compound I | dicamba |
| B-342 | an individualized compound I | diflufenzopyr |
| B-343 | an individualized compound I | quinclorac |
| B-344 | an individualized compound I | quinmerac |
| B-345 | an individualized compound I | mesotrione |
| B-346 | an individualized compound I | saflufenacil |
| B-347 | an individualized compound I | topramezone |

The active compounds specified above as component 2, their preparation, and their action against harmful fungi are known (cf.: http://www.alanwood.net/pesticides/); they are available commercially. The compounds with IUPAC nomenclature, their preparation, and their fungicidal activity are likewise known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

The compositions for mixtures of active compounds are prepared in a known manner in the form of compositions comprising, in addition to the active compounds, a solvent or a solid carrier, for example in the manner stated for compositions of the compounds I.

With respect to the customary ingredients of such compositions, reference is made to what was said about the compositions comprising the compounds I.

The compositions for mixtures of active compounds are suitable as fungicides for controlling harmful fungi. They are distinguished by excellent activity against a broad spectrum of phytopathogenic fungi including soilborne pathogens which originate in particular from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Furthermore, reference is made to what was said about the activity of the compounds I and the compositions comprising the compounds I.

The present invention furthermore provides the use of compounds I and their pharmaceutically acceptable salts for treating diseases, in particular the use of the compounds I as antimycotics. Thus, one embodiment of the invention relates to a medicament comprising at least one compound of the formula I and/or a pharmaceutically acceptable salt thereof. A further embodiment relates to the use of a compound I and/or a pharmaceutically effective salt thereof for preparing an antimycotic.

The present invention also provides the use of compounds I and their pharmaceutically acceptable salts for treating tumors in mammals such as, for example, humans. Thus, one embodiment of the invention relates to the use of a compound I and/or a pharmaceutically acceptable salt thereof for preparing a composition which inhibits the growth of tumors and cancer in mammals. “Cancer” means in particular a malignant tumor, for example breast cancer, cancer of the prostate, lung cancer, cancer of the CNS, melanocarcinomas, ovarial carcinomas or renal cancer, in particular in humans.

The present invention also provides the use of compounds I and their pharmaceutically acceptable salts for treating virus infections, in particular virus infections leading to diseases in warmblooded animals. Thus, one embodiment of the invention relates to the use of a compound I and/or a pharmaceutically acceptable salt thereof for preparing a composition for treating virus infections. The virus diseases to be treated include retrovirus diseases such as, for example: HIV and HTLV, influenza virus, rhinovirus diseases, herpes and the like.

### SYNTHESIS EXAMPLES

With appropriate modification of the starting materials, the procedures given in the synthesis examples below were used to obtain further compounds of the formula I or the precursors thereof, for example for preparing the compounds according to the invention listed in Table E.

### Example 1

### Preparation of 8-(3-chloro-5-trifluoromethylpyridin-2-yloxy)-2,2-dimethyl-4-[1,2,4]triazol-1-yloctan-3-ol (compound I.A1)

### 1.1 Preparation of 8-chloro-2,2-dimethyl-4-[1,2,4]-triazol-1-yloctan-3-one

Tert-butylpinacolone (282.06 g, 1.687 mol) was initially charged in a nitrogen atmosphere in N,N-dimethylformamide (2.2 l), and potassium tert-butoxide (189.29 g, 1.687 mol) was added (exothermal reaction). 1-Bromo-4-chlorobutane dissolved in N,N-dimethylformamide (700 ml) was then added at a temperature between 5 and 10° C., and then the mixture was stirred at room temperature for 16 h. After this time, the reaction mixture was poured onto ice water and extracted with MTBE (3×). The combined organic phases were washed with water, separated off, dried with Na₂SO₄ and freed from the solvent. Residues of bromochlorobutane were removed by distillation from the crude product thus obtained (40-50° C./4 mbar). The product was obtained in the form of a yellow oil and used without any further purification for the next reaction step (yield: 230.0 g, purity 90%, 47%). ¹H NMR (CDCl₃, 400 MHz) δ 8.28 (s, 1H), 8.85 (s, 1H), 5.97 (m, 1H), 3.50 (t, 2H), 2.16-1.63 (m, 4H), 1.42-1.25 (m, 2H), 1.19 (s, 9H).

### 1.2 Preparation of 8-(3-chloro-5-trifluoromethylpyridin-2-yloxy)-2,2-dimethyl-4-[1,2,4]triazol-1-yloctan-3-one

3-Chloro-5-trifluoromethylpyridin-2-ol (1.50 g, 7.38 mmol) together with 8-chloro-2,2-dimethyl-4-[1,2,4]-triazol-1-yloctan-3-one from step 1.1 (1.90 g, 7.38 mmol), potassium carbonate (1.20 g, 7.38 mmol) and potassium iodide (12 mg, 0.06 mmol) was dissolved in DMF (36 ml) and stirred at 180° C. under microwave conditions for 30 min. After this time, the reaction mixture was added to water and extracted 3 times with ethyl acetate. The combined organic phases were washed first with dilute sodium hydroxide solution and then with water, dried, filtered and freed from the solvent. The residue obtained in this manner was purified by column chromatography (silica gel, ethyl acetate/cyclohexane) (1.02 g, 33%).

### 1.3a Preparation of (RR,SS)-8-(3-chloro-5-trifluoromethylpyridin-2-yloxy)-2,2-dimethyl-4-[1,2,4]triazol-1-yloctan-3-ol (compound I.A1a, RR-SS)

8-(3-Chloro-5-trifluoromethylpyridin-2-yloxy)-2,2-dimethyl-4-[1,2,4]triazol-1-yl-octan-3-one from step 1.2 (0.30 g, 0.72 mmol) was dissolved in methanol (10 ml), and sodium borohydride (0.027 g, 0.72 mmol) was added at 0° C. The mixture was allowed to warm to room temperature and stirred for a further 14 h. Saturated ammonium chloride solution was then added, and the reaction mixture was extracted 2 times with ethyl acetate. The combined organic phases were washed with water, dried and filtered. Distillative removal of the solvent gave the target product as the (RR/SS) pair of enantiomers (0.14 g, 46%).

### 1.3b Preparation of (RS,SR)-8-(3-chloro-5-trifluoromethylpyridin-2-yloxy)-2,2-dimethyl-4-[1,2,4]triazol-1-yloctan-3-ol (compound I.A1b, RS-SR)

At −30° C., titanium tetrachloride (1.0 ml of a 1M solution in dichloromethane, 1.0 mmol) was added dropwise to a solution of 8-(3-chloro-5-trifluoromethylpyridin-2-yloxy)-2,2-dimethyl-4-[1,2,4]triazol-1-yloctan-3-one from step 1.2 (0.35 g, 0.84 mmol) in dichloromethane (10 ml). The mixture was stirred at this temperature for 30 min, and tetra-n-butylammonium borohydride (0.11 g, 0.42 mmol, dissolved in 2 ml of dichloromethane) was then added dropwise. The reaction mixture was allowed to warm to room temperature and stirred for a further 14 h. Saturated ammonium chloride solution was then added, and the reaction mixture was extracted 2 times with ethyl acetate. The combined organic phases were washed with water, dried and filtered. Distillative removal of the solvent gave the target compound as the (RS/SR) pair of enantiomers (02.0 g, 57%).

### Example 2

### Preparation of 7-(7-chloroindol-2-yl)-2,2-dimethyl-4-[1,2,4]-triazol-1-ylheptan-3-ol (compound I.A2)

### 2.1 Preparation of 1-(3-bromopropyl)-7-chloro-1H-indole

1,3-Dibromopropane (5.7 g, 28.3 mmol) was initially charged together with 7-chloroindole (3.8 g, 25.3 mmol) in DMF (50 ml) and stirred with finely powdered potassium hydroxide (1.6 g, 28.3 mmol) at room temperature for 14 h. The reaction mixture was then poured into water and extracted 3 times with ether. The combined organic phases were washed with water, dried over Na₂SO₄ and filtered. The product obtained after removal of the solvent was used without any further purification for the next reaction step.

### 2.2 Preparation of 7-(7-chloroindol-2-yl)-2,2-dimethyl-4-[1,2,4]triazol-1-ylheptan-3-one

At 0° C., a mixture of 3,3-dimethyl-1-[1,2,4]triazol-1-ylbutan-2-one (6.1 g, 36.7 mmol) and potassium tert-butoxide (4.1 g, 36.7 mmol) was added dropwise to a solution of 1-(3-bromopropyl)-7-chloro-1H-indole from step 2.1 (5.0 g, 18.3 mmol) and potassium iodide in DMF (25 ml). After the end of the addition, the reaction mixture was stirred at this temperature for another hour, and dichloromethane was then added. The separated organic phase was washed 4 times with water, dried over Na₂SO₄ and filtered. The residue obtained after distillative removal of the solvent was purified by column chromatography (silica gel, ethyl acetate/cyclohexane). The appropriate fractions were pooled, giving the target product in a yield of 2.1 g (32%).

### 2.3a Preparation of (RR, SS)-7-(7-chloroindo1-1-yl)-2,2-dimethyl-4-[1,2,4]triazol-1-ylheptan-3-ol (compound I.A2a, RR-SS)

At 0° C., sodium borohydride (11 mg, 0.3 mmol) was added to a solution of 7-(7-chloroindol-1-yl)-2,2-dimethyl-4-[1,2,4]triazol-1-ylheptan-3-one (100 mg, 0.3 mmol) from step 2.2 in methanol (10 ml), and the mixture was stirred at room temperature for 14 h. Aqueous ammonium chloride solution was then added, and the reaction mixture was extracted 2 times with ethyl acetate. The organic phases were washed with water, dried over Na₂SO₄ and filtered. Distillative removal of the solvent gave the target product as the (RR/SS) pair of enantiomers (84 mg, 83%).

### 2.3b Preparation of (RS, SR)-7-(7-chloroindol-1-yl)-2,2-dimethyl-4-[1,2,4]triazol-1-ylheptan-3-ol (compound I.A2b, RS-SR)

At −30° C., titanium tetrachloride (1.4 ml of a 1M solution in dichloromethane, 1.4 mmol) was added dropwise to a solution of 7-(7-chloroindol-1-yl)-2,2-dimethyl-4-[1,2,4]triazol-1-ylheptan-3-one (500 mg, 1.4 mmol) from step 2.2 in dichloromethane (25 ml). The mixture was stirred at this temperature for 30 min, and tetra-n-butylammonium borohydride (180 mg, 0.7 mmol, dissolved in 3 ml of dichloromethane) was then added. After thawing to room temperature, the mixture was stirred for another 14 h. Saturated ammonium chloride solution was then added, and the reaction mixture was extracted 2 times with ethyl acetate. The combined organic phases were washed with water, dried and filtered. The residue obtained after distillative removal of the solvent was purified by column chromatography (silica gel, ethyl acetate/cyclohexane). The appropriate fractions were pooled, giving the target product in a yield of 206 mg (39%).

### Biological Examples

### A) Greenhouse

### Preparation of Active Compound

The active compounds were prepared separately as a stock solution comprising 25 mg of active compound which was made up to 10 ml using a mixture of acetone and/or DMSO and the emulsifier Uniperol® EL (wetting agent having emulsifying and dispersing action based ethoxylated alkylphenols) in a volume ratio of solvent/emulsifier of 99:1. The mixture was then made up to 100 ml with water. This stock solution was diluted with the solvent/emulsifier/water mixture described to give the concentration of active compounds stated below. Alternatively, the active compounds were used as a commercial finished formulation and diluted with water to the active compound concentration stated.

### Example G1

### Curative Activity Against Soybean Rust Caused by Phakopsora pachyrhizi

Leaves of potted soybean seedlings were inoculated with a spore suspension of soybean rust (Phakopsora pachyrhizi). The pots were then placed in a chamber with high atmospheric humidity (90 to 95%) and at 23 to 27° C. for 24 hours. During this time, the spores germinated and the germ tubes penetrated into the leaf tissue. The infected plants were then sprayed to run off point with the active compound solution described above at the active compound concentration stated below. After the spray coating had dried on, the test plants were cultivated in the greenhouse at temperatures between 23 and 27° C. and 60 to 80% relative atmospheric humidity for 14 days. The extent of the rust fungus development on the leaves was then determined visually in % infection. The plants which had been treated with an aqueous active compound preparation comprising 600 ppm of the active compound I.A9b, I.A22, I.A11, I.A7, I.A17, I.A5, I.A10, I.A1a, I.A21b, I.A21a, I.A2b, I.A19b, I.A1b, I.A20a, I.A2a or I.A19a of Table E showed an infection of at most 15%, whereas the untreated plants were 90% infected. The plants which had been treated with an aqueous active compound preparation comprising 150 ppm of the active compound I.A12 or I.A20b of Table E showed an infection of at most 15%, whereas the untreated plants were 90% infected.

### Example G2

### Protective Activity Against Puccinia recondita on Wheat (Brown Rust of Wheat)

Leaves of potted wheat seedlings were sprayed to run off point using an aqueous suspension having the active compound concentration stated below. The next day, the treated plants were inoculated with a spore suspension of brown rust of wheat (Puccinia recondita). The plants were then placed in a chamber with high atmospheric humidity (90 to 95%) at 20 to 24° C. for 24 hours. During this time, the spores germinated and the germ tubes penetrated into the leaf tissue. The next day, the test plants were returned to the greenhouse and cultivated at temperatures between 20 and 24° C. and 65 to 70% relative atmospheric humidity for a further 7 days. The extent of the rust fungus development on the leaves was then determined visually. The plants which had been treated with an aqueous active compound preparation comprising 600 ppm of the active compound I.A11, I.A7, I.A17, I.A5, I.A10 or I.A1b of Table E showed an infection of at most 15%, whereas the untreated plants were 90% infected.

TABLE E
I.A

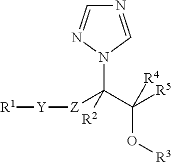

| | | | | | | | Stereochemistry and |
|---|---|---|---|---|---|---|---|
| No. | R¹ | Y—Z | R² | R³ | R⁴ | R⁵ | physical data |
| I.A1 | 3-Cl-5-CF₃-pyridin-2-yl | O—CH₂(CH₂)₂CH₂ | H | H | H | C(CH₃)₃ | a) RR-SS |
| | | | | | | | 3.616 (421) [*] |
| | | | | | | | b) RS-SR |
| | | | | | | | 3.582 (421) [*] |
| I.A2 | 7-Cl-indol-1-yl | CH₂CH₂CH₂ | H | H | H | C(CH₃)₃ | a) RR-SS |
| | | | | | | | 3.259 (361) [*] |
| | | | | | | | b) RS-SR |
| | | | | | | | 122° C. [**] |
| I.A3 | 2-CH₃-pyridin-3-yl | O—CH₂(CH₂)₂CH₂ | H | H | H | C(CH₃)₃ | RS-SR |
| | | | | | | | 1.871 (333) [*] |
| I.A4 | pyridin-4-yl | O—CH₂(CH₂)₂CH₂ | H | H | H | C(CH₃)₃ | RS-SR |
| | | | | | | | 1.789 (319) [*] |
| I.A5 | 2-Cl-pyridin-4-yl | O—CH₂(CH₂)₂CH₂ | H | H | H | C(CH₃)₃ | RS-SR |
| | | | | | | | 2.777 (353) [*] |
| I.A6 | 2,6-di-CH₃-pyridin-4-yl | O—CH₂(CH₂)₂CH₂ | H | H | H | C(CH₃)₃ | RS-SR |
| | | | | | | | 1.896 (347) [*] |
| I.A7 | 2-Cl-pyridin-3-yl | O—CH₂(CH₂)₂CH₂ | H | H | H | C(CH₃)₃ | RS-SR |
| | | | | | | | 2.735 (353) [*] |
| I.A8 | 2-CF₃-pyridin-6-yl | O—CH₂(CH₂)₂CH₂ | H | H | H | C(CH₃)₃ | RS-SR |
| | | | | | | | 3.368 (387) [*] |
| I.A9 | 2-Cl-pyridin-6-yl | O—CH₂(CH₂)₂CH₂ | H | H | H | C(CH₃)₃ | a) RS-SR |
| | | | | | | | 3.299 (353) [*] |
| | | | | | | | b) RR-SS |
| | | | | | | | 3.14 (353) [*] |
| I.A10 | 5-CF₃-pyridin-2-yl | O—CH₂(CH₂)₂CH₂ | H | H | H | C(CH₃)₃ | RS-SR |
| | | | | | | | 3.175 (387) [*] |
| I.A11 | 2-CH₃-pyridin-6-yl | O—CH₂(CH₂)₂CH₂ | H | H | H | C(CH₃)₃ | RS-SR |
| | | | | | | | 2.434 (333) [*] |
| I.A12 | 5-(COOC₂H₅)-pyridin-2-yl | O—CH₂(CH₂)₂CH₂ | H | H | H | C(CH₃)₃ | RS-SR |
| | | | | | | | 3.086 (391) [*] |
| I.A13 | 2-Br-pyridin-3-yl | O—CH₂(CH₂)₂CH₂ | H | H | H | C(CH₃)₃ | RS-SR |
| | | | | | | | 2.68 (398) [*] |
| I.A14 | 5-Cl-pyridin-2-yl | O—CH₂(CH₂)₂CH₂ | H | H | H | C(CH₃)₃ | RS-SR |
| | | | | | | | 2.494 (353) [*] |
| I.A15 | 2-Cl-pyridin-5-yl | O—CH₂(CH₂)₂CH₂ | H | H | H | C(CH₃)₃ | RS-SR |
| | | | | | | | 2.849 (353) [*] |
| I.A16 | 2-CH₃-pyridin-5-yl | O—CH₂(CH₂)₂CH₂ | H | H | H | C(CH₃)₃ | RS-SR |
| | | | | | | | 1.882 (333) [*] |
| I.A17 | 5-Cl-pyridin-5-yl | O—CH₂(CH₂)₂CH₂ | H | H | H | C(CH₃)₃ | RS-SR |
| | | | | | | | 2.835 (353) [*] |
| I.A18 | 2,6-di-CH₃-pyridin-3-yl | O—CH₂(CH₂)₂CH₂ | H | H | H | C(CH₃)₃ | RS-SR |
| | | | | | | | 1.881 (347) [*] |
| I.A19 | 6-Cl-indol-1-yl | CH₂CH₂CH₂ | H | H | H | C(CH₃)₃ | a) RR-SS |
| | | | | | | | 3.272 (361) [*] |
| | | | | | | | b) SR-RS |
| | | | | | | | 91° C. [**] |
| I.A20 | 6-F-indol-1-yl | CH₂CH₂CH₂ | H | H | H | C(CH₃)₃ | a) RR-SS |
| | | | | | | | 3.102 (345) [*] |
| | | | | | | | b) RS-SR |
| | | | | | | | 3.260 (345) [*] |
| I.A21 | 7-F-indol-1-yl | CH₂CH₂CH₂ | H | H | H | C(CH₃)₃ | a) RR-SS |
| | | | | | | | 3.318 (345) [*] |
| | | | | | | | b) RS-SR |
| | | | | | | | 3.269 (345) [*] |
| I.A22 | pyridin-3-yl | O—CH₂(CH₂)₂CH₂ | H | H | H | C(CH₃)₃ | RS-SR |
| | | | | | | | 1.922 (319) [*] |

[*] retention time in min. (HPLC-MS)/m/z (High Performance Liquid Chromatography Mass Spectrometry)
HPLC column: RP-18 column (Chromolith Speed ROD from Merck KgaA, Germany)
mobile phase: acetonitrile + 0.1% trifluoroacetic acid (TFA)/water + 0.1% TFA in a gradient of from 5:95 to 95:5 over the course of 5 minutes at 40° C.
MS: quadrupole electrospray ionization, 80 V (positive mode)
[**] melting point [° C.]

1-13. (canceled)

14. A compound of the formula I 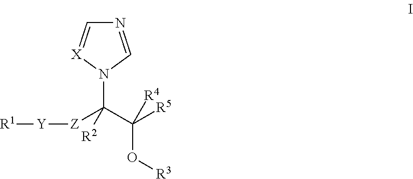
wherein
X is CH or N;
Y is O or a single bond to R¹;
Z is a saturated or partially unsaturated hydrocarbon chain which has two to eight carbon atoms and which, if it is partially unsaturated, comprises one to three double bonds or one or two triple bonds, where Z may comprise one, two, three, four or five substituents R^{Z}, where R^{Z} is as defined below:
R^{Z} is halogen, cyano, nitro, cyanato (OCN), C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-Cₛ-alkynyloxy, C₃-C₈-haloalkynyloxy, C₃-C₈-cycloalkyl, C₃-C₈-halocycloalkyl, C₃-C₈-cycloalkenyl, C₃-C₈-halocyclo-alkenyl, C₃-C₈-cycloalkoxy; C₃-C₆-cycloalkenyloxy, C₁-C₆-alkylene, oxy-C₂-C₄-alkylene, oxy-C₁-C₃-alkyleneoxy, phenoxy, phenyl, heteroaryloxy, heterocyclyloxy, heteroaryl, heterocyclyl, wherein the groups mentioned above, the heteroaryl is an aromatic five-, six- or seven-membered heterocycle and the heterocyclyl is a saturated or partially unsaturated five-, six- or seven-membered heterocycle, each of which contains one, two, three or four heteroatoms selected from the group consisting of O, N and S, or is NA³A⁴, where two radicals Rz attached to the same carbon atom, together with the carbon atom to which they are attached, may also form a C₃-C₆-cycloalkyl ring; wherein A³, A⁴ are as defined below;
R¹ is a three-, four-, five-, six-, seven-, eight-, nine- or ten-membered saturated or partially unsaturated heterocycle or a five-, six-, seven-, eight-, nine- or ten-membered aromatic heterocycle, where the heterocycle contains in each case one, two, three or four heteroatoms selected from the group consisting of O, N and S, wherein the heterocycle is unsubstituted or contains one, two, three, four or five independently selected substituents L, wherein L is as defined below:
L is halogen, cyano, nitro, hydroxyl, cyanato (OCN), C₁-C₈-alkyl, C₁-C₈-haloalkyl, C₂-C₈-alkenyl, C₂-C₈-haloalkenyl, C₂-C₈-alkynyl, C₃-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₃-C₈-haloalkynyl-oxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenoxy, phenyl, heteroaryloxy, heterocyclyloxy, heteroaryl, or heterocyclyl, where, in the groups mentioned above, the heteroaryl is an aromatic five-, six- or seven-membered heterocycle and the heterocyclyl is a saturated or partially unsaturated five-, six- or seven-membered heterocycle, each of which contains one, two, three or four heteroatoms selected from the group consisting of O, N and S, wherein n, A¹, A², A³, A⁴ are as defined below:
n is 0, 1 or 2;
A¹ is hydrogen, hydroxyl, C₁-C₈-alkyl, C₁-C₈-haloalkyl, amino, C₁-C₈-alkylamino, di-C₁-C₈-alkylamino, phenyl, phenylamino or phenyl-C₁-C₈-alkylamino;
A² is one of the groups mentioned for A¹ or is C₂-C₈-alkenyl, C₂-C₈-haloalkenyl, C₂-C₈-alkynyl, C₃-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₃-C₈-haloalkynyloxy, C₃-C₈-cycloalkyl, C₃-C₈-halocycloalkyl, C₃-C₈-cycloalkoxy or C₃-C₈-halocycloalkoxy;
A³,A⁴ independently of one another are hydrogen, C₁-C₈-alkyl, C₁-C₈-haloalkyl, C₂-Cralkenyl, C₂-C₈-haloalkenyl, C₂-C₈-alkynyl, C₃-C₈-haloalkynyl, C₃-C₈-cycloalkyl, C₃-C₈-halocycloalkyl, C₃-C₈-cycloalkenyl or C₃-C₈-halocycloalkenyl, phenyl or 5- or six-membered heteroaryl having one, two, three or four heteroatoms selected from the group consisting of O, N and S in the heterocycle;
the aliphatic and/or alicyclic and/or aromatic groups of the radical definitions of L for their part may carry one, two, three or four identical or different groups R^{L}:
R^{L} is halogen, hydroxyl, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₆-alkylene, oxy-C₂-C₄-alkylene, oxy-C₁-C₃-alkyleneoxy, C₁-C₈-alkylcarbonyl, C₁-C₈-alkylcarbonyloxy, C₁-C₈-alkoxycarbonyl, amino, C₁-C₈-alkylamino, or di-C₁-C₈-alkylamino;
R² is hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₄-C₁₀-alkadienyl, C₄-C₁₀-haloalkadienyl, C₃-C₁₀-cycloalkyl, C₃-C₁₀-halocycloalkyl, C₃-C₁₀-cycloalkenyl, or C₃-C₁₀-halocycloalkenyl;
R³ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₄-C₁₀-alkadienyl, C₄-C₁₀-haloalkadienyl, C₃-C₁₀-cycloalkyl, C₃-C₁₀-halocycloalkyl, C₃-C₁₀-cycloalkenyl, C₃-C₁₀-halocyclo-alkenyl, carboxyl, formyl, Si(A⁵A⁶A⁷), C(O)Rⁿ, C(O)ORⁿ, C(S)ORⁿ, C(O)SRⁿ, C(S)SRⁿ, C(NR^{A})SRⁿ, C(S)Rⁿ, C(NRⁿ)N—NA³A⁴, C(NRⁿ)R^{A}, C(NRⁿ)OR^{A}, C(O)NA³A⁴, C(S)NA³A⁴ or S(═O)ₙA¹; where
Rⁿ is C₁-C₈-alkyl, C₃-C₈-alkenyl, C₃-C₈-alkynyl, C₃-C₆-cycloalkyl, C₃-C₆-cycloalkenyl or phenyl;
R^{A} is C₁-C₈-alkyl, C₃-C₈-alkenyl, C₃-C₈-alkynyl, C₃-C₆-cycloalkyl, C₃-C₆-cycloalkenyl or phenyl;
A⁵, A⁶, A⁷ independently of one another are C₁-C₁₀-alkyl, C₃-C₈-alkenyl, C₃-C₈-alkynyl, C₃-C₆-cycloalkyl, C₃-C₆-cycloalkenyl or phenyl;
where Rⁿ, R^{A}, A⁵, A⁶ and A⁷ are, unless indicated otherwise, independently of one another unsubstituted or substituted by one, two, three, four or five L, as defined above;
R⁴ is hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₃-C₁₀-haloalkynyl, C₄-C₁₀-alkadienyl, C₄-C₁₀-haloalkadienyl, C₃-C₁₀-cycloalkyl, C₃-C₁₀-halocycloalkyl, C₃-C₁₀-cycloalkenyl, or C₃-C₁₀-halocycloalkenyl;
R², R³, R⁴ are, unless indicated otherwise, independently of one another unsubstituted or substituted by one, two, three, four or five L, as defined above;
R⁵ is C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, C₃-C₁₀-cycloalkenyl, C₃-C₁₀-halocycloalkenyl, phenyl, five-, six- or seven-membered heteroaryl which contains 1, 2, 3 or 4 heteroatoms selected from the group consisting of O, N and S or five-, six- or seven-membered saturated or partially unsaturated heterocyclyl which contains 1, 2, 3 or 4 heteroatoms selected from the group consisting of O, N and S, where R⁵ may contain one, two, three, four, five or six independently selected substituents L, as defined above;
and agriculturally acceptable salts thereof.

15. The compound of claim 14, wherein Z is a group Z¹: 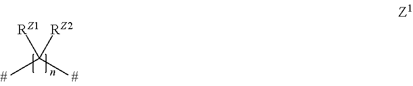
wherein # are the points of attachment, n is 2, 3, 4, 5 or 6 and R^{z1} and R^{z2} are in each case selected independently of one another from the group consisting of hydrogen and R^{Z}, as defined in claim 14.

16. The compound of claim 14, wherein Z is a group Z² 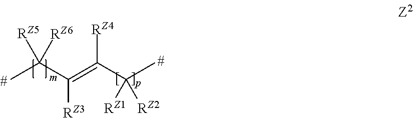
wherein # are the points of attachment, m and p are each 0, 1 or 2, where m+p≧1, and R^{Z1}, R^{Z2}, R^{Z3}, R^{Z4}, R^{Z5} and R^{Z6} are, in each case, selected independently of one another from the group consisting of hydrogen and R^{Z}, as defined in claim 14.

17. The compound of claim 14, wherein X is N.

18. The compound of claim 14, wherein Y is O.

19. The compound of claim 14, wherein Y is a bond.

20. The compound of claim 14, wherein R¹ is a 5-, 6-, 7-, 8- or 9-membered aromatic heterocycle which is unsubstituted or substituted by one, two, three or four independently selected L and which contains 1, 2, 3 or 4 heteroatoms selected from the group consisting of O, N and S.

21. An active compound composition comprising at least one compound as defined in claim 14, and/or a salt thereof and at least one further fungicidally, insecticidally and/or herbicidally active compound.

22. The active compound composition of claim 21, further comprising at least one solid or liquid carrier.

23. A seed treated with at least one compound as defined in claim 14, and/or an agriculturally acceptable salt thereof.

24. A method for controlling phytopathogenic fungi wherein the fungi or the materials, plants, the soil or seed to be protected from fungal attack are treated with an effective amount of a compoundas defined in claim 14, or an agriculturally acceptable salt thereof.

25. The method of claim 24, wherein Z is a group Z¹: 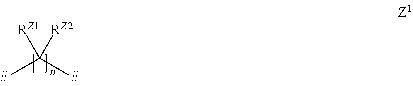
wherein # are the points of attachment, n is 2, 3, 4, 5 or 6 and R^{z1} and R^{z2} are in each case selected independently of one another from the group consisting of hydrogen and R^{Z}, as defined in claim 14.

26. The method of claim 24, wherein Z is a group Z² 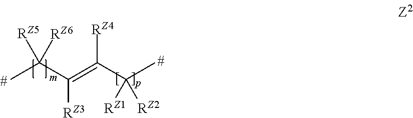
wherein # are the points of attachment, m and p are each 0, 1 or 2, where m+p≧1, and R^{Z1}, R^{Z2}, R^{Z3}, R^{Z4}, R^{Z5} and R^{Z6} are, in each case, selected independently of one another from the group consisting of hydrogen and R^{Z}, as defined in claim 14.

27. The method of claim 24, wherein X is N.

28. The method of claim 24, wherein Y is O.

29. The method of claim 24, wherein Y is a bond.

30. The method of claim 24, wherein R¹ is a 5-, 6-, 7-, 8- or 9-membered aromatic heterocycle which is unsubstituted or substituted by one, two, three or four independently selected L and which contains 1, 2, 3 or 4 heteroatoms selected from the group consisting of O, N and S.
